(19) United States
(12) Patent Application Publication
(10) Pub. No.: US 2026/0335217 A1
(43) Pub. Date: Oct. 29, 2026

(54) LIPOSOME-CONTAINING COSMETIC COMPOSITIONS

(71) Applicant: LipoSphere Ltd., Givat Shmuel (IL)

(72) Inventors: Ronit GOLDBERG, Givat Shmuel (IL); Sabrina JAHN, Hindhead (GB); Asaf LEVIN, Petah Tikva (IL)

(73) Assignee: LipoSphere Ltd., Givat Shmuel (IL)

(21) Appl. No.: 19/107,092

(22) PCT Filed: Aug. 30, 2023
(86) PCT No.: PCT/IL2023/050926

§ 371 Date: Feb. 27, 2025

Related U.S. Application Data
(60) Provisional application No. 63/402,088, filed on Aug. 30, 2022.

(51) Int. Cl. A61K 8/81; A61K 8/14; A61Q 19/00

(52) U.S. Cl. CPC A61K 8/8152; A61K 8/14; A61Q 19/007

(57) ABSTRACT

A cosmetic or cosmeceutical composition (or formulation) comprising a liposome or a plurality of liposomes and a cosmetically acceptable carrier is provided. At least a portion of the liposomes are made of one or more bilayer-forming lipid(s) and a polymeric compound as described and defined in the instant specification and claims. Skin care products comprising the cosmetic or cosmeceutical composition and uses thereof are also provided.

### RELATED APPLICATION

This application claims the benefit of priority under 35 USC § 119(e) of U.S. Provisional Patent Application No. 63/402,088 filed on Aug. 30, 2022.

This application also relates to U.S. Provisional Patent Application No. 63/402,097, filed Aug. 30, 2022, by the present assignee, the contents of which are incorporated by reference as if fully set forth herein.

### FIELD AND BACKGROUND OF THE INVENTION

The present invention, in some embodiments thereof, relates to cosmetics, and, more particularly, but not exclusively, to cosmetic compositions and products having incorporated therein liposomes formed of a lipid bilayer forming material and a polymeric material.

The human skin, which is the body's largest organ, protects against germs, regulates body temperature and enables touch (tactile) sensations. Three layers of tissue make up the skin: Hypodermis—the bottom layer, Dermis—the middle layer, and the Epidermis—the top layer. The stratum corneum is the target skin layer for many cosmetic products.

Ceramide is the main component of the stratum corneum of the epidermis layer of human skin. Together with cholesterol and saturated fatty acids, ceramide creates a water-impermeable, protective organ to prevent excessive water loss due to evaporation as well as a barrier against the entry of microorganisms. The stratum corneum is composed of 50% ceramides, 25% cholesterol, and 15% free fatty acids. These lipids form bilayers with a thickness of 4-5 nm. A particular group of ceramides contains a C36-side chain, which extends into the neighbouring bilayer, thereby “nailing” them together. Key components of the extracellular lipid lamellae of the stratum corneum are ultra-long chain (C28-C36) ceramides. With aging there is a decline in ceramide and cholesterol in the stratum corneum of humans. See, for example, Hill J R and Wertz P W (2009) Lipids, 44 (3): 291-295; Garidel P et al. (2010) Biophysical Chemistry, 150 (1-3):144-156; Feingold K R (2007) Journal of Lipid Research, 48 (12): 2531-2546; Jennemann R et al. (2012) Human Molecular Genetics. 21 (3): 586-608; and Popa I et al. (2010) International Journal of Cosmetic Science, 32 (3): 225-232.

The degree of hydration of the skin, particularly the stratum corneum (the outmost barrier of the epidermis), is directly related to its appearance (i.e. smoothness of texture) and is also considered as an indicator of its state of health. Moreover, dry skin is more vulnerable to external aggressions. Further, the skin water content is a key factor for skin permeability to topically applied substances, wherein increased hydration of the stratum corneum usually enhances percutaneous flux of the substance [Esposito et al. (2007) International Journal of Cosmetic Science, 29(1), pp. 39-47]. Thus, maintaining the water content of the skin barrier is essential for maintaining the functionality and aesthetic appearance of the skin.

Lipids play a decisive role in creation and maintaining the barrier function of the stratum corneum. During the conversion of keratinocytes to corneocytes and the migration to the top of the skin, a tremendous activity of anabolism and catabolism of lipidic material is going on. As one of the consequences of the phospholipase activity in the stratum corneum, the phospholipid percentage of the lipid composition decreases dramatically from the basal layer (about 50%) to the stratum corneum (<5%), and the fatty acid content in the stratum corneum significantly increases.

Studies have shown that the stratum corneum is not a homogeneous matrix in itself; yet, it forms a very heterogeneous structure and contains besides corneocytes and multilamellar lipid layers a variety of other biologically active substances.

A lipid is a biomolecule that is soluble in non-polar solvents. Non-polar solvents are typically hydrocarbons used to dissolve substances that are essentially water-immiscible, including fatty acids, waxes, sterols, fat-soluble vitamins (such as vitamins A, D, E, and K), monoglycerides, diglycerides, triglycerides, and phospholipids.

Fatty acids and lipids have been used as components of various cosmetic products due to their biocompatibility and ability to protect and improve skin barriers and appearance. For example, lipids have a high adhesiveness to the skin that leads to a film formation onto the skin and thus assists in the repair of defects in the natural skin barrier and reinforcement of thin natural lipid film. This re-creation of the skin barrier has an anti-pollution effect. Moreover, lipids lead to occlusion created by the particle film on the skin which in turn increases the skin hydration and maintains proper living conditions for the basal keratinocytes cells that constitute most of the epidermis layer.

In cosmetics products, the biodegradable phospholipids can be used as in formulations for suspensions, oil-in-water and water-in-oil emulsions and mixed micelles.

Phospholipids are a class of lipids whose molecule has a hydrophilic “head” containing a phosphate group and two hydrophobic “tails” derived from fatty acids, joined by an alcohol residue (usually a glycerol molecule). In typical membrane phospholipids, for instance in phosphatidylcholine (PC), the phosphate group is further esterified with additional alcohol, and in phosphatidylethanolamine (PE) with ethanolamine. Depending upon the structure of the polar region and pH of the medium, PC and PE are zwitterionic and have a neutral charge at pH values of about 7, whereas other phospholipids can be negatively charged.

When mixed with an aqueous phase, phospholipids can form various structures, depending on the number and type of fatty acids esterified to the glycerol backbone and the ratio of the surface areas occupied by the hydrophilic and lipophilic part of the phospholipid molecule. Diacylphospholipids (like PC and PE) having a cylindrical shape are typically organized as lipid bilayers (liposomes) with the hydrophobic tails lined up against one another and the hydrophilic head group facing the water on both sides. The bilayer membrane of such a liposome resembles the basic structure of cellular membranes, rendering it both biocompatible and having a beneficial interaction with skin cells.

Phospholipids have been used in cosmetics, typically as surface active agents (e.g., emulsifiers), and/or for enhancing skin penetration, and have also been used as bilayer lipid-forming materials.

Phospholipid polymers have been used to modify the surface structure of cell membranes. Phospholipid polymers suppress adsorption of proteins, feature high biocompatibility, and can retain moisture retention on the surface of the skin, making them an appealing raw material in cosmetics.

2-Methacryloyloxyethyl phosphorylcholine (MPC) is a biomimetic biocompatible material with a structure similar to the skin cell membrane, and has been used in various fields such as cosmetics and personal care products, contact lens, contact lens storage and cleaning solutions, medical devices, textiles, cell culture equipment, etc.

Co-polymers, including block co-polymers, made of MPC and hydrophobic methacrylate monomers that feature, for example, alkyl pendant groups (e.g., butyl or higher alkyls), have been described as improving moisture retention capability of the skin, and have been introduced to various cosmetic formulations.

International Patent Application Publication No. WO 2017/109784 describes polymeric compounds comprising a lipid moiety and an ionic polymeric moiety, such as a pMPC (poly(O-(2-methacryloyloxyethyl)phosphorylcholine)) moiety, as well as bilayers and liposomes comprising such a polymeric compound in combination with a bilayer-forming lipid. The bilayers and/or liposomes comprising such polymeric compounds are described as being useful for reducing a friction coefficient of a surface and/or for inhibiting biofilm formation.

Additional background art includes International Patent Application publications WO 2016/051413 and WO 2018/150429; FR 2774286; EP 3662890; JP 6679308; and U.S. Pat. Nos. 5,744,145, 11,260,021, 5,653,966 and 8,956,668.

### SUMMARY OF THE INVENTION

According to an aspect of some embodiments of the present invention there is provided a cosmetic or cosmeceutical composition (or formulation) comprising a liposome and a cosmetically acceptable carrier, wherein the liposome comprises:
a) at least one bilayer-forming lipid; and
b) a polymeric compound having the general formula I:

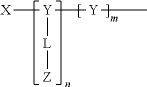

wherein:
m is zero or a positive integer;
n is an integer which is at least 2, at least 5, preferably at least 10 (e.g., of from 10 to 200);
Y is a backbone unit which forms a polymeric backbone of the polymeric compound;
L is absent or is a linking moiety;
Z has the general formula II:

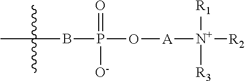

wherein:
the curved line denotes an attachment point to the respective Y backbone unit;
A is a substituted or unsubstituted hydrocarbon;
B is an oxygen atom or is absent;
R₁-R₃ are each independently selected from the group consisting of hydrogen, alkyl, cycloalkyl, heteroalicyclic, aryl and heteroaryl; and
X is a lipid moiety represented by Formula IV:

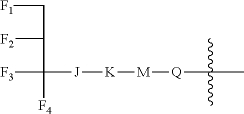

wherein:
F₁, F₂, F₃ and F₄ are each independently selected from the group consisting of hydrogen, alkyl, alkenyl, alkynyl, acyl, alkoxy, thioalkoxy, carboxy, thiocarboxy, wherein at least one of F₁, F₂, F₃ and F₄ is not hydrogen and is of at least 10 carbon atoms in length;
J is —O—P(═O)(OH)—O— or absent;
K is a substituted or unsubstituted hydrocarbon from 1 to 10 carbon atoms in length or absent;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent,
wherein when M is absent, Q is also absent, and when J is absent, M is not absent,
provided that:
when J is —O—P(═O)(OH)—O—, M is other than amido and/or Q comprises an aryl moiety.

According to some of any of the embodiments described herein, at least one of F₁, F₂, F₃ and F₄ is an alkoxy, thioalkoxy, acyl or carboxy of at least 10 carbon atoms in length.

According to some of any of the embodiments described herein, at least one of F₁, F₂, F₃ and F₄ is derived from a fatty acid selected from the group consisting of lauroyl, myristoyl, palmitoyl, stearoyl, palmitoleoyl, oleoyl, and linoleoyl.

According to some of any of the embodiments described herein, M is carboxy.

According to some of any of the embodiments described herein, K is an alkyl.

According to some of any of the embodiments described herein, J is —P(═O)(OH)—O—; M is amido; and Q is a hydrocarbon substituted by at least one aryl (e.g., phenyl).

According to some of any of the embodiments described herein, Q is a methylene substituted by at least one aryl.

According to some of any of the embodiments described herein, J is absent.

According to some of any of the embodiments described herein, J and K are each absent.

According to some of any of the embodiments described herein, J and K are each absent and M is carboxy.

According to some of any of the embodiments described herein, at least one, or at least two, of F₁, F₂, F₃ and F₄ is independently the thioalkoxy.

According to some of any of the embodiments described herein, at least one, or at least two, of F₁, F₂, F₃ and F₄ is independently the carboxy.

According to some of any of the embodiments described herein, at least one or both of F₁ and F₂ is the carboxy and at least one of F₃ and F₄ is an alkyl.

According to some of any of the embodiments described herein, Y is a substituted or unsubstituted alkylene unit.

According to some of any of the embodiments described herein, Y is a substituted or unsubstituted ethylene unit.

According to some of any of the embodiments described herein, Y has the formula —CR₄R₅—CR₆D-, wherein:
when Y is a backbone unit which is not attached to the L or the Z, D is R₇; and when Y is a backbone unit which is attached to the L or the Z, D is a covalent bond or a linking group attaching Y to the L or the Z, the linking group being selected from the group consisting of —O—, —S—, alkylene, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino; and
R₄-R₇ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino.

According to some of any of the embodiments described herein, R₄-R₇ are each independently selected from hydrogen and alkyl.

According to some of any of the embodiments described herein, R₄ and R₅ are each hydrogen.

According to some of any of the embodiments described herein, R₆ is hydrogen.

According to some of any of the embodiments described herein, the linking group is selected from the group consisting of —O—, —C(═O)O—, —C(═O)NH— and phenylene.

According to some of any of the embodiments described herein, the linking group is —C(═O)O—.

According to some of any of the embodiments described herein, L is a substituted or unsubstituted hydrocarbon from 1 to 10 carbon atoms in length.

According to some of any of the embodiments described herein, L is a substituted or unsubstituted ethylene group.

According to some of any of the embodiments described herein, B is an oxygen atom.

According to some of any of the embodiments described herein, A is a substituted or unsubstituted hydrocarbon from 1 to 4 carbon atoms in length.

According to some of any of the embodiments described herein, A is a substituted or unsubstituted ethylene group.

According to some of any of the embodiments described herein, R₁-R₃ are each independently hydrogen or C₁₋₄-alkyl.

According to some of any of the embodiments described herein, R₁-R₃ are each methyl.

According to some of any of the embodiments described herein, n ranges from 10 to 200.

According to some of any of the embodiments described herein, n is at least 30.

According to some of any of the embodiments described herein, n ranges from 30 to 70, or from 30 to 60.

According to some of any of the embodiments described herein, n is at least 50, or at least 60.

According to some of any of the embodiments described herein, n ranges from 50 to 150, or from 60 to 150, or from 50 to 80, or from 60 to 80.

According to some of any of the embodiments described herein, n ranges from 60 to 80.

According to some of any of the embodiments described herein, n is at least 80, or ranges from 80 to 150, or from 80 to 120.

According to some of any of the embodiments described herein, m ranges from 0 to 50.

According to some of any of the embodiments described herein, at least a portion of the backbone units Y, the L and/or the Z comprise at least one targeting moiety, as described herein.

According to some of any of the embodiments described herein, a mol ratio of the bilayer-forming lipid and the polymeric compound is in a range of from 5:1 to 5,000:1, or from 10:1 to 1,000:1, or 10:1 to 100:1 or from 10:1 to 50:1, or from 100:1 to 200:1. According to some of any of the embodiments described herein, at least a portion of the liposomes comprises a plurality of liposomes as described herein in any of the respective embodiments and any combination thereof.

According to some of any of the embodiments described herein, a polydispersity index of the liposomes is lower than 1, or lower than 0.8, or lower than 0.6.

According to some of any of the embodiments described herein, a mean diameter of the liposomes is in a range of from 50 to 5,000 nm, or from 500 to 5,000 nm, or from 500 to 3500 nm, or from 200 to 2,000 nm, or from 200 to 1,000 nm, or from 400 to 1,000 nm, or from 400 to 800 nm.

According to some of any of the embodiments described herein, an amount of the bilayer-forming lipid in the composition in a range of from 0.1 to 5, % by weight of the total weight of the composition.

According to some of any of the embodiments described herein, a weight ratio of the bilayer-forming lipid and the polymeric compound is in a range of from 1:1 to 3,000:1, or from 1:1 to 1,000:1, or from 1:1 to 500:1, or from 1:1 to 300:1, or from 1:1 to 100:1, or from 1:1 to 50:1, or from 5:1 to 50:1, or from 5:1 to 30:1.

According to some of any of the embodiments described herein, the cosmetic or cosmeceutical composition is formulated for topical application.

According to some of any of the embodiments described herein, the carrier comprises an aqueous liquid.

According to some of any of the embodiments described herein, the liposome is, or the plurality of liposomes are, included in the aqueous liquid.

According to some of any of the embodiments described herein, the carrier forms a composition in a form of a cream, an ointment, a gel, a lotion, a soap, a shampoo, a water-in-oil emulsion, an oil-in-water emulation, a water-in-oil-in water emulation, an oil-in water-in oil emulsion.

According to some of any of the embodiments described herein, a total amount of the bilayer-forming lipid and the polymeric compound is in a range of from 0.1 to 10, or from 0.1 to 5, % by weight of the total weight of the composition.

According to some of any of the embodiments described herein, the composition is in a form of a gel and a total amount of the bilayer-forming lipid and the polymeric compound is in a range of from 1 to 10, or from 1 to 5, % by weight of the total weight of the composition.

According to some of any of the embodiments described herein, the composition is in a form of a cream or an emulsion and a total amount of the bilayer-forming lipid and the polymeric compound is in a range of from 0.1 to 5, or from 0.1 to 1, % by weight of the total weight of the composition.

According to an aspect of some embodiments of the present invention there is provided a skin care product comprising the cosmetic or cosmeceutical composition as described herein in any of the respective embodiments and any combination thereof.

According to an aspect of some embodiments of the present invention there is provided a composition as described herein in any of the respective embodiments and any combination thereof or the skin care product as described herein in any of the respective embodiments and any combination thereof, for use in skin hydration.

According to an aspect of some embodiments of the present invention there is provided a composition as described herein in any of the respective embodiments and any combination thereof or the skin care product as described herein in any of the respective embodiments and any combination thereof, for use in treating a damaged keratinous tissue.

According to an aspect of some embodiments of the present invention there is provided a method of performing a cosmetic care in a subject in need thereof, the method comprising applying to the skin of the subject an effective amount of the composition as described herein in any of the respective embodiments and any combination thereof or the skin care product as described herein in any of the respective embodiments and any combination thereof, thereby performing the cosmetic care.

Unless otherwise defined, all technical and/or scientific terms used herein have the same meaning as commonly understood by one of ordinary skill in the art to which the invention pertains.

Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a scheme showing an exemplary synthetic protocol for preparing an exemplary polymeric compound (lipid-polymer conjugate; LPC) according to some embodiments of the present invention.

FIGS. 2A-B present graphs showing the corneometry (hydration) of the IM22-777 gel formulation as a function of time in comparison with a glycerin reference IM22-587 (FIG. 2A) and in comparison with various lipid standards (IM22-743 and IM22-736) (FIG. 2B).

FIG. 2C presents the TEWL of IM22-777 as a function of time, in comparison with various standards.

FIGS. 3A-B present graphs showing the corneometry (hydration) of the IM22-778 gel formulation as a function of time in comparison with a glycerin reference IM22-587 (FIG. 3A) and in comparison with various lipid standards (IM22-743 and IM22-736) (FIG. 3B).

FIG. 3C presents the TEWL of IM22-778 as a function of time, in comparison with various standards.

FIGS. 4A-B present graphs showing the corneometry (hydration) of the IM22-754 gel formulation as a function of time in comparison with a glycerin reference IM22-587 (FIG. 4A) and in comparison with various lipid standards (IM22-743 and IM22-736) (FIG. 4B).

FIG. 4C presents the TEWL of IM22-754 as a function of time, in comparison with various standards.

FIGS. 5A-B present graphs showing the corneometry (hydration) of the IM22-744 gel formulation as a function of time in comparison with a glycerin reference IM22-587 (FIG. 5A) and in comparison with various lipid standards (IM22-743 and IM22-736) (FIG. 5B).

FIG. 6 presents graphs showing the corneometry (hydration) of the IM22-732 gel formulation as a function of time in comparison with a various lipid standards (IM22-743 and IM22-736).

FIGS. 7A-B present graphs showing the corneometry (hydration) of the IM22-782 gel formulation as a function of time in comparison with a glycerin reference IM22-587 (FIG. 7A) and in comparison with various lipid standards (IM22-743 and IM22-736) (FIG. 7B).

FIG. 7C presents the TEWL of IM22-782 as a function of time, in comparison with various standards.

FIGS. 8A-B present graphs showing the corneometry (hydration) of the IM22-780 gel formulation as a function of time in comparison with a glycerin reference IM22-587 (FIG. 8A) and in comparison with various lipid standards (IM22-743 and IM22-736) (FIG. 2B).

FIG. 9A presents comparative plots showing the magnetization signal from different gels on porcine skin, normalized to compensate for the different signal-decaying processes shown. Fitted dotted and dashed lines correspond to the unbound- and embedded-water regimes. The vertical dashed lines represent the calculation for the expected completion time of evaporation.

FIG. 9B presents a magnified portion of FIG. 9A, zooming mainly on the central embedded water regime shown in FIG. 9A, comprising 3 formulae, as shown by the dashed lines. The DMPC-LPC consistently has the smallest slope, meaning the lowest nominal shrinkage rate, indicating that it persists its embedded water molecules for the longest time.

FIG. 10A presents comparative plots showing the magnetization signal from different gels on porcine skin (absolute values).

FIG. 10B presents comparative plots showing the decay of echo signal along 120 π-pulses after the π/2-pulse. Each point is an average of 100 such sequence iterations

FIG. 11 presents comparative plots showing the accumulated and averaged mass loss after gel deposition as measured by the analytical balance. Blue circles—5% Glycerine; red squares—DMPC; green triangles—DMPC-LPC.

### DESCRIPTION OF SPECIFIC EMBODIMENTS OF THE INVENTION

The present invention, in some embodiments thereof, relates to cosmetics, and, more particularly, but not exclusively, to cosmetic compositions and products having incorporated therein liposomes formed of a lipid bilayer forming material and a polymeric material.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While investigating liposome-containing solutions, the present inventors have found that incorporating liposomes comprising a lipid-derived polymeric compound, also referred to herein as “a polymeric compound” “a lipid-polymer conjugate” or abbreviated as “LPC”, and optionally a bilayer-forming lipid, in topical compositions or formulations, successfully increases skin hydration and also increases the duration of the hydration layer on the skin (see, the Examples section which follows). The present inventors have conceived using these liposomes as an advantageous component of cosmetic compositions, formulations, or products, particularly in such compositions, formulations or products that advantageously utilize phospholipids, or phospholipid-containing polymeric materials, in addition to, or instead of, phospholipids or phospholipid-containing polymeric materials that are currently commonly included in cosmetic compositions, formulations or products.

Consequently, specific embodiments of the present teachings suggest cosmetic formulations (e.g., for topical application) comprising these lipid-derived polymeric compounds and/or liposomes comprising same.

According to some of any of the embodiments described herein, the polymeric compounds are as described in U.S. Provisional Patent Application No. 63/402,097, filed Aug. 30, 2022 and/or in a co-filed PCT International Patent Application having Attorney's Docket No. 97558, which claims the benefit of priority of U.S. Provisional Patent Application No. 63/402,097.

According to an aspect of some embodiments of the present invention, there is provided a cosmetic or cosmeceutical composition comprising a liposome as defined herein in any of the respective embodiments and any combination thereof and a cosmetically acceptable carrier.

As used herein throughout, the term “cosmetic” with regard to any of the compositions and methods (and any other aspect) disclosed herein, describes compositions that enhance the appearance or odor of the human body.

In some embodiments, the term “cosmetic” follows the definition regulatory authorities worldwide. For example, in some embodiments, the term “cosmetic” follows the definition of the U.S. Food and Drug Administration (FDA), as being “intended to be applied to the human body for cleansing, beautifying, promoting attractiveness, or altering the appearance without affecting the body's structure or functions”.

As used herein, the phrase “cosmeceutical composition” describes a composition for topical administration as a cosmetic, which comprises a biologically active ingredient, that is, a compound that exhibits a biological activity such as, for example, anti-inflammatory, anti-bacterial and/or anti-oxidation activity.

The phrase “keratinous material” or “keratinous substrate” or “keratinous tissue”, which are used interchangeably herein, means, in some embodiments of the present invention, a material, substrate or tissue that is enriched with keratin, including, for example, nails, hair and skin, and especially bodily areas like the face, cheeks, hands, body, legs, around the eyes, the eyelids and the lips.

“Skin” means the outermost protective covering of mammals that is composed of cells such as keratinocytes, fibroblasts and melanocytes. Skin includes an outer epidermal layer and an underlying dermal layer. Skin may also include hair and nails as well as other types of cells commonly associated with skin, such as, for example, myocytes, Merkel cells, Langerhans cells, macrophages, stem cells, sebocytes, nerve cells and adipocytes.

As used herein and in the art, the term “liposome” refers to an artificially prepared vesicle comprising a bilayer composed of molecules of an amphiphilic lipid. In an aqueous medium, the bilayer is typically configured such that hydrophilic moieties of the amphiphilic lipid are exposed to the medium at both surfaces of the bilayer, whereas lipophilic moieties of the lipid are located in the internal portion of the bilayer, and therefore less exposed to the medium. Examples of liposomes which may be used in any one of the embodiments described herein include, without limitation, small unilamellar vesicles, large unilamellar vesicles and multilamellar vesicles.

As described herein, the liposome according to embodiments comprises, inter alia, a polymeric compound.

### Polymeric Compounds (LPCs):

According to an aspect of some embodiments of the invention, there are provided polymeric compounds collectively represented by Formula I:

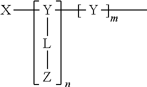

wherein:
m is zero or a positive integer;
n is an integer which is at least 2, at least 5, preferably at least 10 (e.g., of from 10 to 200);
Y is a backbone unit which forms a polymeric backbone of the polymeric compound;
X is a lipid moiety as described herein in any of the respective embodiments;
L is absent or is a linking moiety; and
Z has the general Formula II:

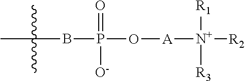

wherein:
the dashed (curved) line denotes an attachment point to the respective Y backbone unit or to the linking moiety L, if present;
A is a substituted or unsubstituted hydrocarbon;
B is an oxygen atom or is absent; and
R₁-R₃ are each independently selected from the group consisting of hydrogen, alkyl, cycloalkyl, heteroalicyclic, aryl and heteroaryl,
as described in more detail herein below.

Formula I may also be described herein simply as:

X—[Y(-L-Z)]n[Y]m-
which is to be regarded as interchangeable with the schematic depiction hereinabove, wherein X is a lipid moiety conjugated to the —[Y(-L-Z)]n[Y]m- polymeric moiety.

### Polymeric Moiety:

Herein, the term “polymeric” refers to a compound having at least 2 repeating units (and more preferably at least 3 repeating units), the repeating units being identical or similar. It is to be appreciated that the compound of general Formula I is by definition polymeric when n is at least 2, as it comprises at least 2 of the backbone units represented by Y.

Herein, the phrase “polymeric moiety” refers to the portion of the polymeric compound (according to any of the embodiments described herein relating to general Formula I) which has the general Formula Ia:

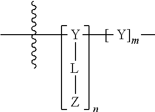

wherein m, n, Y, L and Z are as defined herein for general Formula I, and the dashed (curved) line represents an attachment point to the X lipid moiety.

Formula Ia may also be described herein simply as:

—[Y(-L-Z)]n[Y]m-
which is to be regarded as interchangeable with the schematic depiction hereinabove.

Herein, the phrase “polymeric compound” further encompasses compounds having a “polymeric moiety” as described herein having at least one unit (e.g., according to Formula Ia wherein n is at least 1), provided that the lipid moiety described herein (e.g., the lipid moiety represented by X) has a similar unit. For example, when the lipid moiety comprises a phosphate group (e.g., the lipid moiety is a glycerophospholipid moiety), and a single unit of the polymeric moiety has a phosphate group, the two phosphate groups may be regarded as repeating units.

In preferred embodiments, however, n is at least 2, such that the polymeric moiety per se has at least two units. In some embodiments, n is at least 3.

As used herein, the term “backbone unit” refers to a repeating unit, wherein linkage of a plurality of the repeating unit (e.g., sequential linkage) forms a polymeric backbone. A plurality of linked repeating units per se is also referred to herein as a “polymeric backbone”. A polymeric moiety as described herein can comprise a plurality of repeating backbone units which identical to one another, and thereby form a homopolymeric moiety, or, alternatively, can comprise two or more types of repeating backbone units, which can be linked to one another randomly or in a certain order (e.g., as two or more blocks, or in alternating order), and thereby form a copolymer moiety.

As shown in Formulae I and Ia, L and Z together form a pendant group of at least a portion of the backbone units, which group is referred to herein for brevity simply as the “pendant group”.

Each backbone unit Y with a pendant group (i.e., a unit represented by Y(-L-Z), the number of which is represented by the variable n) and each backbone unit Y without a pendant group (the number of which is represented by the variable m) is also referred to herein as a “monomeric unit”.

A backbone unit may optionally be a unit of a polymerizable monomer or polymerizable moiety of a monomer. A wide variety of polymerizable monomers and moieties will be known to the skilled person, and the structure of the units of such monomers which result upon polymerization (e.g., monomeric units) will also be known to the skilled person.

A “unit of a polymerizable monomer” refers to a modified form of a polymerizable monomer and/or a portion of a polymerizable monomer that remains after polymerization.

A portion of a polymerizable monomer may be formed, for example, by a condensation reaction, e.g., wherein at least one atom or group (e.g., a hydrogen atom or hydroxyl group) in the monomer, and optionally at least two atoms or groups (e.g., a hydrogen atom and a hydroxyl group) in the monomer, is replaced with a covalent bond with another polymerizable monomer.

A modified form of a polymerizable monomer may be formed, for example, by ring-opening (wherein a covalent bond between two atoms in a ring is broken, and each of the two atoms optionally becomes linked to another polymerizable monomer); and/or by adding to an unsaturated bond, wherein an unsaturated bond between two adjacent atoms is broken (e.g., conversion of an unsaturated double bond to a saturated bond, or conversion of an unsaturated triple bond to an unsaturated double bond) and the two atoms optionally each become linked to another polymerizable monomer.

A modified form of a polymerizable monomer may consist essentially of the same atoms as the original monomer, for example, different merely in the rearrangement of covalent bonds, or alternatively, may have a different atomic composition, for example, wherein polymerization includes a condensation reaction (e.g., as described herein).

Examples of backbone units include, without limitation, substituted or unsubstituted hydrocarbons (which may form a substituted or unsubstituted hydrocarbon backbone), such as alkylene units; hydroxycarboxylic acid units (which may form a polyester backbone), e.g., glycolate, lactate, hydroxybutyrate, hydroxyvalerate, hydroxycaproate and hydroxybenzoate units; dicarboxylic acid units (which may form a polyester backbone in combination with a diol and/or a polyamide in combination with a diamine), e.g., adipate, succinate, terephthalate and naphthalene dicarboxylic acid units; diol units (which may form a polyether backbone, or form a polyester backbone in combination with a dicarboxylic acid), e.g., ethylene glycol, 1,2-propanediol, 1,3-propanediol, 1,4-butanediol, and bisphenol A units; diamine units (which may form a polyamide backbone in combination with a dicarboxylic acid), e.g., para-phenylene diamine and alkylene diamines such hexylene diamine; carbamate units (which may form a polyurethane backbone); amino acid residues (which may form a polypeptide backbone); and saccharide moieties (which may form a polysaccharide backbone).

In some embodiments of any of the embodiments described herein, Y is a substituted or unsubstituted alkylene unit.

In some embodiments, Y is a substituted or unsubstituted ethylene unit, that is, an alkylene unit 2 atoms in length.

Polymeric backbones wherein Y is a substituted or unsubstituted ethylene unit may optionally be a polymeric backbone such as formed by polymerizing ethylene (CH₂═CH₂) and/or substituted derivatives thereof (also referred to herein as “vinyl monomers”). Such polymerization is a very well-studied procedure, and one of ordinary skill in the art will be aware of numerous techniques for effecting such polymerization.

It is to be understood that any embodiments described herein relating to a polymeric backbone formed by a polymerization encompass any polymeric backbone having a structure which can be formed by such polymerization, regardless of whether the polymeric backbone was formed in practice by such polymerization (or any other type of polymerization).

As is well known in the art, the unsaturated bond of ethylene and substituted ethylene derivatives becomes saturated upon polymerization, such that the backbone units in a polymeric backbone formed by the polymerization are saturated, although they may be referred to as units of an unsaturated compound (e.g., a “vinyl monomer” or “olefin monomer”) to which they are analogous.

Polymers which can be formed from unsaturated monomers such as vinyl monomers and olefin monomers are also referred to by the terms “polyvinyl” and “polyolefin”, respectively.

Herein, an “unsubstituted” alkylene unit (e.g., ethylene unit) refers to an alkylene unit which does not have any substituent other than the pendant group discussed herein (represented as (-L-Z)). That is, an alkylene unit attached to the aforementioned pendant group is considered unsubstituted if there are no substituents at any other positions on the alkylene unit.

In some embodiments of any of the embodiments described herein, Y has the formula —CR₄R₅—CR₆D-.

When Y is a backbone unit which is not attached to L or Z (i.e., to a pendant group as described herein), D is R₇ (an end group, as defined herein); and when Y is a backbone unit which is attached to L or Z, D is a covalent bond or a linking group attaching Y to L or Z. The linking group may optionally be —O—, —S—, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

R₄-R₇ are each independently hydrogen, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

Herein throughout, the phrase “linking group” describes a group (e.g., a substituent) that is attached to two or more moieties in the compound.

Herein throughput, the phrase “end group” describes a group (e.g., a substituent) that is attached to a single moiety in the compound via one atom thereof.

When each of R₄-R₆ is hydrogen, and D is a covalent bond or linking group, Y is an unsubstituted ethylene group attached (via D) to a pendant group described herein.

When each of R₄-R₇ is hydrogen (and D is R₇), Y is an unsubstituted ethylene group which is not attached to a pendant group described herein.

In some embodiments of any of the embodiments described herein, R₄ and R₅ are each hydrogen. Such embodiments include polymeric backbones formed from many widely used vinyl monomers (including ethylene), including, for example, olefins (e.g., ethylene, propylene, 1-butylene, isobutylene, 4-methyl-1-pentene), vinyl chloride, styrene, vinyl acetate, acrylonitrile, acrylate and derivatives thereof (e.g., acrylate esters, acrylamides), and methacrylate and derivatives thereof (e.g., methacrylate esters, methacrylamides).

In some embodiments of any of the embodiments described herein, R₆ is hydrogen. In some such embodiments, R₄ and R₅ are each hydrogen.

In some embodiments of any of the embodiments described herein, R₆ is methyl. In some such embodiments, R₄ and R₅ are each hydrogen. In some such embodiments, the backbone unit is a unit of methacrylate or a derivative thereof (e.g., methacrylate ester, methacrylamide).

In some embodiments of any of the embodiments described herein, the linking group represented by the variable D is —O—, —C(═O)O—, —C(═O)NH— or phenylene. In exemplary embodiments, D is —C(═O)O—.

For example, the backbone unit may optionally be a vinyl alcohol derivative (e.g., an ester or ether of a vinyl alcohol unit) when D is —O—; an acrylate or methacrylate derivative (e.g., an ester of an acrylate or methacrylate unit) when D is —C(═O)O—; an acrylamide or methacrylamide unit when D is —C(═O)NH—; and/or a styrene derivative (e.g., a substituted styrene unit) when D is phenylene.

In some embodiments of any of the embodiments described herein, L is a substituted or unsubstituted hydrocarbon from 1 to 10 carbon atoms in length. In some embodiments, the hydrocarbon is unsubstituted. In some embodiments, the hydrocarbon is a linear, unsubstituted hydrocarbon, that is, —(CH₂)ᵢ— wherein i is an integer from 1 to 10.

In some embodiments of any of the embodiments described herein, L is a substituted or unsubstituted ethylene group. In some embodiments, L is an unsubstituted ethylene group (—CH₂CH₂—).

In some embodiments of any of the embodiments described herein, B is an oxygen atom.

In some such embodiments, L is a hydrocarbon according to any of the respective embodiments described herein (i.e., L is not absent), and Z is a phosphate group attached to L.

In some embodiments of any of the embodiments described herein, B is absent. In some such embodiments, L is a hydrocarbon according to any of the respective embodiments described herein (i.e., L is not absent), and Z is a phosphonate group attached to L. In some embodiments, L is also absent, such that the phosphorus atom of Formula TT is attached directly to Y.

In some embodiments of any of the embodiments described herein, A is a substituted or unsubstituted hydrocarbon from 1 to 4 carbon atoms in length.

In some embodiments of any of the embodiments described herein, A is an unsubstituted hydrocarbon. In some such embodiments, the unsubstituted hydrocarbon is from 1 to 4 carbon atoms in length. In some embodiments, the hydrocarbon is a linear, unsubstituted hydrocarbon, that is, —(CH₂)ⱼ— wherein j is an integer from 1 to 4.

In some embodiments of any of the embodiments described herein, A is a substituted or unsubstituted ethylene group.

In some embodiments of any of the embodiments described herein, A is an unsubstituted ethylene group (—CH₂CH₂—). In such embodiments, the moiety having general Formula II (represented by the variable Z) is similar or identical to a phosphoethanolamine or phosphocholine moiety. Phosphoethanolamine and phosphocholine moieties are present in many naturally occurring compounds (e.g., phosphatidylcholines, phosphatidylethanolamines).

In some embodiments of any of the embodiments described herein, A is an ethylene group substituted by a C-carboxy group. In some embodiments, the C-carboxy is attached to the carbon atom adjacent to the nitrogen atom depicted in Formula II (rather than the carbon atom attached to the depicted oxygen atom). In such embodiments, the moiety having general Formula II (represented by the variable Z) is similar or identical to a phosphoserine moiety. Phosphoserine is present in many naturally occurring compounds (e.g., phosphatidylserines).

Without being bound by any particular theory, it is believed that moieties similar or identical to naturally occurring moieties such as phosphocholine, phosphoethanolamine and/or phosphoserine may be particularly biocompatible.

In some embodiments of any of the embodiments described herein, R₁-R₃ (the substituents of the nitrogen atom depicted in general Formula II) are each independently hydrogen or C₁₋₄-alkyl. In some embodiments, R₁-R₃ are each independently hydrogen or methyl. In some embodiments, R₁-R₃ are each methyl. In some such embodiments, R₁-R₃ are each hydrogen.

The variable n may be regarded as representing a number of backbone units (represented by the variable Y) which are substituted by the pendant group represented by (-L-Z), and the variable m may be regarded as representing a number of backbone units which are not substituted by such a pendant group. The sum n+m may be regarded as representing the total number of backbone units in the polymeric backbone. The ratio n/(n+m) may be regarded as representing the fraction of backbone units which are substituted by the pendant group represented by (-L-Z).

In some embodiments of any of the embodiments described herein, the percentage of backbone units (represented by the variable Y) which are substituted by the pendant group represented by (-L-Z) (as represented by the formula 100%*n/(n+m)) is at least 20%. In some embodiments, the percentage of backbone units substituted by the aforementioned pendant group is at least 30%. In some embodiments, the percentage of backbone units substituted by the aforementioned pendant group is at least 40%. In some embodiments, the percentage of backbone units substituted by the aforementioned pendant group is at least 50%. In some embodiments, the percentage of backbone units substituted by the aforementioned pendant group is at least 60%. In some embodiments, the percentage of backbone units substituted by the aforementioned pendant group is at least 70%. In some embodiments, the percentage of backbone units substituted by the aforementioned pendant group is at least 80%. In some embodiments, the percentage of backbone units substituted by the aforementioned pendant group is at least 90%. In some embodiments, the percentage of backbone units substituted by the aforementioned pendant group is at least 95%. In some embodiments, the percentage of backbone units substituted by the aforementioned pendant group is at least 98%.

In some embodiments of any of the embodiments described herein, m is 0, such that each of the backbone units (represented by the variable Y) is substituted by the pendant group represented by (-L-Z).

In some embodiments of any of the embodiments described herein, n is at least 5. In some embodiments, n is at least 10. In some embodiments, n is at least 15.

In some embodiments of any of the embodiments described herein, n is in a range of from 2 to 1,000, including any intermediate value and subranges therebetween. In some embodiments of any of the embodiments described herein, n is in a range of from 2 to 500, including any intermediate value and subranges therebetween. In some embodiments of any of the embodiments described herein, n is in a range of from 2 to 200, including any intermediate value and subranges therebetween. In some embodiments of any of the embodiments described herein, n is in a range of from 2 to 100, including any intermediate value and subranges therebetween. In some embodiments of any of the embodiments described herein, n is in a range of from 2 to 50, including any intermediate value and subranges therebetween. In some such embodiments, m is 0.

In some embodiments of any of the embodiments described herein, n is in a range of from 3 to 1,000, including any intermediate value and subranges therebetween. In some embodiments of any of the embodiments described herein, n is in a range of from 3 to 500, including any intermediate value and subranges therebetween. In some embodiments of any of the embodiments described herein, n is in a range of from 3 to 200, including any intermediate value and subranges therebetween. In some embodiments of any of the embodiments described herein, n is in a range of from 3 to 100, including any intermediate value and subranges therebetween. In some embodiments of any of the embodiments described herein, n is in a range of from 3 to 50, including any intermediate value and subranges therebetween. In some embodiments of any of the embodiments described herein, n is in a range of from 5 to 50, including any intermediate value and subranges therebetween. In some embodiments of any of the embodiments described herein, n is in a range of from 10 to 50, including any intermediate value and subranges therebetween. In some embodiments of any of the embodiments described herein, n is in a range of from 10 to 25, including any intermediate value and subranges therebetween. In some such embodiments, m is 0.

In some embodiments of any of the embodiments described herein, n is in a range of from 10 to 200, including any intermediate value and subranges therebetween. In some embodiments of any of the embodiments described herein, n is in a range of from 10 to 180, including any intermediate value and subranges therebetween. In some embodiments of any of the embodiments described herein, n is in a range of from 10 to 150, including any intermediate value and subranges therebetween. In some embodiments of any of the embodiments described herein, n is in a range of from 10 to 120, including any intermediate value and subranges therebetween 0. In some such embodiments, m is 0.

In some embodiments of any of the embodiments described herein, n is at least 30.

In some embodiments of any of the embodiments described herein, n is in a range of from 30 to 200, including any intermediate value and subranges therebetween. In some embodiments of any of the embodiments described herein, n is in a range of from 30 to 180, including any intermediate value and subranges therebetween. In some embodiments of any of the embodiments described herein, n is in a range of from 30 to 150, including any intermediate value and subranges therebetween. In some embodiments of any of the embodiments described herein, n is in a range of from 30 to 120, including any intermediate value and subranges therebetween 0. In some such embodiments, m is 0.

In some embodiments of any of the embodiments described herein, n is in a range of from 30 to 70, including any intermediate value and subranges therebetween. In some embodiments of any of the embodiments described herein, n is in a range of from 35 to 65, including any intermediate value and subranges therebetween. In some such embodiments, m is 0.

In some embodiments of any of the embodiments described herein, n is at least 50, or at least 60, or at least 80.

In some embodiments of any of the embodiments described herein, n is in a range of from 50 to 200, including any intermediate value and subranges therebetween. In some embodiments of any of the embodiments described herein, n is in a range of from 50 to 180, including any intermediate value and subranges therebetween. In some embodiments of any of the embodiments described herein, n is in a range of from 50 to 150, including any intermediate value and subranges therebetween. In some embodiments of any of the embodiments described herein, n is in a range of from 50 to 120, including any intermediate value and subranges therebetween. In some embodiments of any of the embodiments described herein, n is in a range of from 80 to 120, including any intermediate value and subranges therebetween. In some embodiments of any of the embodiments described herein, n is in a range of from 50 to 100, including any intermediate value and subranges therebetween. In some embodiments of any of the embodiments described herein, n is in a range of from 50 to 80, including any intermediate value and subranges therebetween. In some such embodiments, m is 0.

In some embodiments of any of the embodiments described herein, when n is lower than 80, or lower than 70, or lower than 50, or lower than 30, or is in a range of from 10 to 50, or from 30 to 60, or from 30 to 80, or from 30 to 70, or from 50 to 80, as described herein in any of the respective embodiments, the polymeric compound is referred to herein as “short” or “S”.

In some embodiments of any of the embodiments described herein, when n is higher than 80, or is higher than 100, or is in a range of from 50 to 150, or from 50 to 120, or from 80 to 150, or from 80 to 120, as described herein in any of the respective embodiments, the polymeric compound is referred to herein as “long” or “L”.

In some embodiments of any of the embodiments described herein, n is in a range of from 10 to 50, including any intermediate value and subranges therebetween. In some such embodiments, m is 0.

In some embodiments of any of the embodiments described herein, n is in a range of from 50 to 80, including any intermediate value and subranges therebetween. In some such embodiments, m is 0.

In some embodiments of any of the embodiments described herein, n is in a range of from 80 to 120, including any intermediate value and subranges therebetween. In some such embodiments, m is 0.

In some embodiments of any of the embodiments described herein, m is in a range of from 0 to 1,000, including any intermediate value and subranges therebetween. In some such embodiments, n is in a range of from 2 to 1,000, including any intermediate value and subranges therebetween, such that the total number of backbone units is in a range of from 2 to 2,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 500, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 200, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 100, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 50, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 20, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some embodiments, n is in a range of from 50 to 80, including any intermediate value and subranges therebetween. In some embodiments, n is in a range of from 80 to 120, including any intermediate value and subranges therebetween.

In some embodiments of any of the embodiments described herein, m is in a range of from 0 to 10, including any intermediate value and subranges therebetween. In some such embodiments, n is in a range of from 2 to 1,000, including any intermediate value and subranges therebetween. In some such embodiments, n is in a range of from 3 to 1,000, including any intermediate value and subranges therebetween. In some embodiments, n is in a range of from 3 to 500, including any intermediate value and subranges therebetween. In some embodiments, n is in a range of from 3 to 200, including any intermediate value and subranges therebetween. In some embodiments, n is in a range of from 3 to 100, including any intermediate value and subranges therebetween. In some embodiments, n is in a range of from 3 to 50, including any intermediate value and subranges therebetween. In some embodiments, n is in a range of from 5 to 50, including any intermediate value and subranges therebetween. In some embodiments, n is in a range of from 10 to 50, including any intermediate value and subranges therebetween. In some of any of the embodiments described herein for m, n is in a range of from 30 to 70, as described herein in any of the respective embodiments, or represents a short polymeric moiety, as described herein. In some such embodiments, n is in a range of from 50 to 80, including any intermediate value and subranges therebetween.

In some of any of the embodiments described herein for m, n is in a range of from 80 to 120, as described herein in any of the respective embodiments, or represents a long polymeric moiety, as described herein.

In some embodiments of any of the embodiments described herein, the backbone unit Y which is substituted by the pendant group represented by (-L-Z) is the same as the backbone unit Y which is not substituted by the pendant group (e.g., when m is at least 1). In alternative embodiments, at least a portion of the backbone units Y which are substituted by the pendant group are different than a portion of the backbone unit Y which is not substituted by the pendant group (e.g., when m is at least 1).

In some embodiments of any of the embodiments described herein, the plurality (indicated by the variable n) of backbone units Y which are substituted by the pendant group represented by (-L-Z) are the same as each other. In alternative embodiments, at least a portion of the plurality of backbone units Y which are substituted by the pendant group represented by (-L-Z) are different from a second portion of the plurality of backbone units Y which are substituted by the pendant group.

In some embodiments of any of the embodiments described herein, the plurality (indicated by the variable n) of pendant groups (-L-Z) attached to the plurality of backbone units Y are the same as each other. In alternative embodiments, at least a portion of the pendant groups (-L-Z) attached to the plurality of backbone units Y are different from each other (e.g., differ in the identity of any one or more of A, B, R₁, R₂, R₃ and L).

In any of the embodiments described herein wherein more than one backbone unit Y is not substituted by the pendant group described herein (i.e., when m is greater than 1), the plurality (indicated by the variable m) of backbone units Y which are not substituted by the pendant group are the same as each other. In alternative embodiments wherein m is larger than 1, at least a portion of the backbone units Y which are not substituted by the pendant group described herein, are different from at least a second portion of the plurality of backbone units Y which are not substituted by the pendant group.

The number of types of backbone units substituted by the pendant group, the number of types of backbone units not substituted by the pendant group (if any such units are present), and/or the number of types of pendant group in the polymeric moiety may each independently be any number (e.g., 1, 2, 3, 4, 5, 6, 7, 8, 9, 10 or more).

In some embodiments of any of the embodiments described herein, the polymeric moiety is a copolymer moiety, that is, the polymeric moiety comprises at least two different types of monomeric unit. In some such embodiments, the different types of monomeric units differ in whether they comprise the pendant group (-L-Z) according to any of the respective embodiments described herein (e.g., when m is at least 1), and/or the different types of monomeric units differ in the type of backbone unit Y, and/or the different types of monomeric units differ in the type of pendant group (-L-Z).

For example, in some embodiments of any of the embodiments described herein the backbone unit Y in each of the Y(-L-Z) units may optionally be the same or different, while the L and Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In some embodiments of any of the embodiments described herein the L moiety in each of the Y(-L-Z) units may optionally be the same or different, while the backbone units Y and the Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In some embodiments of any of the embodiments described herein the Z moiety in each of the Y(-L-Z) units may optionally be the same or different, while the backbone units Y and the Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In any of the embodiments described herein wherein the polymeric moiety is a copolymer moiety, any two or more different types of monomeric unit may be distributed randomly or non-randomly throughout the polymeric moiety. When different types of monomeric unit are distributed non-randomly, the copolymer may be one characterized by any non-random distribution, for example, an alternating copolymer, a periodic copolymer, and/or a block copolymer.

In some of any of the embodiments described herein, the polymeric moiety, which is attached at one of its termini to the lipid moiety X, can have various terminal groups at the other terminus (i.e., at the terminus near the backbone unit Y without a pendant group, wherein m is at least 1; or at the other terminus near the backbone unit Y with a pendant group, wherein m is zero).

The terminal group can be an intrinsic terminal group, derived from the monomers used to form the polymeric compound and/or from the process used to polymerize the monomers, or can be otherwise conjugated to, or generated within, the terminus of the polymeric moiety. For example, the terminal group can be hydrogen, halo, alkyl, hydroxy, carboxy and the like, or can be a targeting moiety, as described in further detail hereinafter. In some of any of the embodiments described herein, the terminal group is hydrogen or halo. In some of any of the embodiments described herein the terminal group is derived from the initiator used to form the polymeric compound as described herein in any of the respective embodiments and exemplified in the Examples section that follows, and in some of these embodiments the terminal group is a halo (e.g., chloro or bromo).

In some of any of the embodiments described herein, the terminal group is a functional group that is suitable for electron transfer radical polymerization, e.g., variable Ri in Formula V, as described herein in any of the respective embodiments.

In some embodiments of any of the embodiments described herein, when n is lower than 60, or lower than 50, or lower than 30, as described herein in any of the respective embodiments, the polymeric compound is referred to herein as “short” or “S”.

In some embodiments of any of the embodiments described herein, when n is lower than 80 but higher than 50 or higher than 60, as described herein in any of the respective embodiments, the polymeric compound is referred to herein as “medium”.

In some embodiments of any of the embodiments described herein, when n is higher than 80, or higher than 90, or higher than 100, as described herein in any of the respective embodiments, the polymeric compound is referred to herein as “long” or “L”.

In some embodiments of any of the embodiments described herein, n is in a range of from 50 to 120, including any intermediate value and subranges therebetween. In some such embodiments, m is 0.

In some embodiments of any of the embodiments described herein, n is in a range of from 60 to 120, including any intermediate value and subranges therebetween. In some such embodiments, m is 0.

In some embodiments of any of the embodiments described herein, n is in a range of from 80 to 120, or from 90 to 120, or from 100 to 120, or from 100 to 110, including any intermediate value and subranges therebetween. In some such embodiments, m is 0.

In some of any of the embodiments described herein for m, n is in a range of from 80 to 120, as described herein in any of the respective embodiments, or represents a long polymeric moiety, as described herein.

### Lipid Moiety:

The lipid moiety (represented by the variable X in Formula I herein) according to any of the embodiments in this section may be attached to a polymeric moiety according to any of the embodiments described in the section herein relating to the polymeric moiety.

The lipid moiety may optionally be derived from any lipid known in the art (including, but not limited to, a naturally occurring lipid). Derivation of the lipid moiety from the lipid may optionally consist of substituting a hydrogen atom at any position of the lipid with the polymeric moiety represented in general Formula I by [Y(-L-Z)]n[Y]m (i.e., the polymeric moiety represented by general Formula Ia).

In some embodiments of any of the embodiments described herein, the lipid moiety (according to any of the respective embodiments described herein) is attached to a Y(-L-Z) unit (according to any of the embodiments described herein relating to Y, L and/or Z), that is, backbone unit substituted by the pendant group described herein (e.g., rather than a backbone unit not substituted by the pendant group).

Alternatively or additionally, in some embodiments of any of the embodiments described herein wherein m is at least 1, the lipid moiety (according to any of the respective embodiments described herein) may optionally be attached to a backbone unit (Y) which is not substituted by a pendant group described herein (e.g., rather than attached to a backbone unit substituted by the pendant group). For example, the polymeric moiety may optionally be a copolymer wherein the identity of the backbone unit attached to the lipid moiety varies randomly between molecules.

Thus, the depiction of X in Formula I as being attached to a backbone unit substituted by a pendant group (i.e., Y-(L-Z)) rather than to an unsubstituted backbone unit Y is arbitrary, and is not intended to be limiting.

In some embodiments of any of the embodiments described herein, the lipid moiety is a moiety of a lipid which is a fatty acid, a monoglyceride, a diglyceride, a triglyceride, a glycerophospholipid, a sphingolipid, or a sterol. In some embodiments, the lipid is a glycerophospholipid.

In some embodiments of any of the embodiments described herein, the lipid moiety comprises at least one fatty acid moiety (e.g., an acyl group derived from a fatty acid). The fatty acid moiety may be derived from a saturated or unsaturated fatty acid. For example, the lipid moiety may consist of a fatty acid moiety, or be a monoglyceride moiety comprising one fatty acid moiety, a diglyceride moiety comprising two fatty acid moieties, or a triglyceride moiety comprising three fatty acid moieties.

Examples of fatty acid moieties which may optionally be comprised by the lipid moiety include, without limitation, lauroyl, myristoyl, palmitoyl, stearoyl, palmitoleoyl, oleoyl, and linoleoyl.

Suitable examples of glycerophospholipids include, without limitation, a phosphatidyl ethanolamine, a phosphatidyl serine, a phosphatidyl glycerol and a phosphatidyl inositol.

In some embodiments of any of the embodiments described herein, the lipid moiety represented by the variable X has the general Formula I is represented by Formula IV:

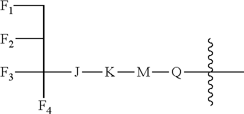

wherein:
the dashed (curved) line denotes an attachment point to the polymeric backbone (i.e., via the respective Y backbone unit);
F₁, F₂, F₃ and F₄ are each independently selected from the group consisting of hydrogen, alkyl, alkenyl, alkynyl, acyl, alkoxy, thioalkoxy, carboxy, thiocarboxy, wherein at least one of F₁, F₂, F₃ and F₄ is not hydrogen and is of at least 10 carbon atoms in length;
J is —O—P(═O)(OH)—O— or absent;
K is a substituted or unsubstituted hydrocarbon from 1 to 10 carbon atoms in length or absent;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent, wherein when M is absent, Q is also absent.

Q is attached to a backbone unit of the polymeric backbone according to any of the respective embodiments described herein, or alternatively, when Q is absent, M is attached to the aforementioned backbone unit.

When M is absent, Q is also absent, and K is attached to a backbone unit of the polymeric backbone according to any of the respective embodiments described herein.

In some embodiments of any of the embodiments described herein for Formula IV, when J is absent, M is not absent.

In some embodiments of any of the embodiments described herein for Formula IV, when J is —O—P(═O)(OH)—O—, M is other than amido and/or Q comprises an aryl moiety.

In some embodiments of any of the embodiments described herein for Formula IV, at least one of F₁, F₂, F₃ and F₄ is an alkoxy, thioalkoxy, acyl or carboxy, preferably of at least 10 carbon atoms in length, for example, of from 8 to 40, or of from 10 to 40, or of from 10 to 30, carbon atoms in length.

In some such embodiments, the alkoxy, thioalkoxy, acyl and/or carboxy has an alkyl moiety that is derived from a fatty acid acyl, as described herein, and is, for example, lauroyl, myristoyl, palmitoyl, stearoyl, palmitoleoyl, oleoyl, and linoleoyl.

In some embodiments of any of the embodiments described herein for Formula IV, at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

In some embodiments of any of the embodiments described herein, at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein, M is other than amido.

According to some of any of the embodiments described herein, M is carboxy.

According to some of any of the embodiments described herein, M is carboxy and at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

According to some of any of the embodiments described herein, M is carboxy and at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein, J is absent.

According to some of any of the embodiments described herein, J is absent and M is other than amido.

According to some of any of the embodiments described herein, J is absent and M is carboxy.

According to some of any of the embodiments described herein, J is absent, and at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

According to some of any of the embodiments described herein, J is absent, M is carboxy, and at least one, or at least two, of F₁, F₂, F₃ and F₄ is independently a thioalkoxy. In some of these embodiments, the thioalkoxy is of at least 10 carbon atoms in length, for example, of from 8 to 40, or of from 10 to 40, or of from 10 to 30, carbon atoms in length. In exemplary embodiments, the alkyl is 15 carbon atoms in length and is derived from palmitic acid. In some of these embodiments, the thioalkoxy has an alkyl group that is of lauric acid, myristic acid, palmitic acid, stearic acid, oleic acid or linoleic acid. In some of these embodiments, each of F₁ and F₂ is independently a thioalkoxy as described herein, and can be the same or different, preferably the same. In some of these embodiments, F₃ and F₄ are each hydrogen.

According to some of any of the embodiments described herein, J is absent, and at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein, J is absent, M is carboxy, and at least one, or at least two, of F₁, F₂, F₃ and F₄ is independently a carboxy. In some of these embodiments, the carboxy is of at least 10 carbon atoms in length, for example, of from 8 to 40, or of from 10 to 40, or of from 10 to 30, carbon atoms in length. In exemplary embodiments, the carboxy is 16 carbon atoms in length and is derived from palmitic acid. In some of these embodiments, the carboxy has an alkyl group that is of lauric acid, myristic acid, palmitic acid, stearic acid, oleic acid or linoleic acid. In some of these embodiments, at least one or both of F₁ and F₂ is a carboxy as described herein in any of the respective embodiments. In some of these embodiments, both of F₁ and F₂ is a carboxy as described herein in any of the respective embodiments, which can be the same or different and is preferably the same. In some of any of these embodiments, at least one of F₃ and F₄ is an alkyl, which can be the same or different. In some such embodiments, the alkyl is a short alkyl of 1 to 6, or 1 to 4 carbon atoms in length, for example, methyl. Alternatively, each of F₃ and F₄ is hydrogen.

According to some of any of the embodiments described herein for Formula IV when J is absent, Q is —C(CH₃)₂—.

According to some of any of the embodiments described herein for Formula IV when J is absent and M is carboxy, Q is —C(CH₃)₂—.

Herein, the length of the hydrocarbon represented by the variable K refers to the number of atoms separating J and M (i.e., along the shortest path between J and M) as depicted in Formula IV, in cases when J is not absent, or the number of atoms separating M and the lipid skeleton formed of F₁, F₂, F₃ and F₄.

When K is a substituted hydrocarbon, M may be attached to a carbon atom of the hydrocarbon per se, or be attached to a substituent of the hydrocarbon.

In some embodiments, K is an all-carbon hydrocarbon.

In some embodiments, K is an unsubstituted hydrocarbon.

In some embodiments, K is an unsubstituted all-carbon hydrocarbon.

In some of any of these embodiments, K is an alkyl (an alkylene chain or linking group), preferably unsubstituted, and optionally being a short alkyl or alkylene of 1 to 6, or 1 to 4, or 1 to 2, carbon atoms in length.

According to some of any of the embodiments described herein, K is absent.

According to some of any of the embodiments described herein, J is absent, as described herein in any of the respective embodiments, and K is absent. In some of these embodiments, M is carboxy.

According to some of any of the embodiments described herein, Q is a hydrocarbon substituted by at least one aryl (e.g., phenyl).

In some of these embodiments, Q is a hydrocarbon which is an all-carbon hydrocarbon, and is some of these embodiments the hydrocarbon is alkyl (an alkylene linking group), preferably a short alkyl (or alkylene) of from 1 to 6, or from 1 to 4, preferably 1 or 2, carbon atoms in length, substituted by at least one aryl (e.g., phenyl).

According to some of any of the embodiments described herein, Q is a methylene substituted by at least one aryl (e.g., phenyl).

According to some of any of the embodiments described herein, J is —P(═O)(OH)—O—; M is amido; and Q is a hydrocarbon substituted by at least one aryl (e.g., phenyl), as described herein in any of the respective embodiments and any combination thereof.

According to some of any of the embodiments described herein, J is —P(═O)(OH)—O—; M is amido; and Q is a methylene substituted by at least one aryl (e.g., phenyl).

In some embodiments of any of the embodiments described herein for Formula I, the lipid moiety represented by the variable X has the general Formula III:

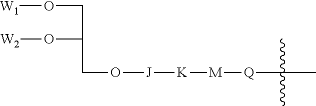

wherein:
the dashed (curved) line denotes an attachment point to the respective Y backbone unit;
W₁ and W₂ are each independently hydrogen, alkyl, alkenyl, alkynyl or acyl, wherein at least one of W₁ and W₂ is not hydrogen;
J is —P(═O)(OH)—O—, or J is absent (such that K is attached directly to the depicted oxygen atom of a glycerol moiety);
K is a substituted or unsubstituted hydrocarbon from 1 to 10 carbon atoms in length;
M is a linking group which is —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, or sulfonamide, or M is absent (such that K is attached directly to Q); and
Q is a substituted or unsubstituted hydrocarbon from 1 to 10 carbon atoms in length, or Q is absent.

Q is attached to a backbone unit of the polymeric backbone according to any of the respective embodiments described herein, or alternatively, when Q is absent, M is attached to the aforementioned backbone unit.

When M is absent, Q is also absent, and K is attached to a backbone unit of the polymeric backbone according to any of the respective embodiments described herein.

In some embodiments of any of the embodiments described herein for Formula III, one of W₁ and W₂ is hydrogen and the other is not hydrogen.

In some embodiments of any of the embodiments described herein for Formula III, neither W₁ nor W₂ is hydrogen.

In some embodiments of any of the embodiments described herein for Formula III, at least one of W₁ and W₂ is an alkyl, alkenyl, alkynyl or acyl, which is from 10 to 30 carbon atoms in length. In some embodiments, each of W₁ and W₂ is from 10 to 30 carbon atoms in length.

Examples of acyl groups which may optionally serve independently as W₁ and/or W₂ include, without limitation, lauroyl, myristoyl, palmitoyl, stearoyl, palmitoleoyl, oleoyl, and linoleoyl.

In some embodiments of any of the embodiments described herein for Formula III, J is —P(═O)(OH)—O— (e.g., the lipid moiety is a glycerophospholipid).

Herein, the length of the hydrocarbon represented by the variable K refers to the number of atoms separating J and M (i.e., along the shortest path between J and M) as depicted in Formula III.

When K is a substituted hydrocarbon, M may be attached to a carbon atom of the hydrocarbon per se, or be attached to a substituent of the hydrocarbon.

In some embodiments of any of the embodiments described herein for Formula III, K is an acyl moiety (e.g., —C(═O)—C(CH₃)₂—). In some such embodiments, J is absent, such that K is attached directly to the depicted oxygen atom of a glycerol moiety. In some such embodiments, K comprises a carbonyl linking group (—C(═O)—), which attaches to the oxygen atom of a glycerol moiety via an ester bond.

In some embodiments of any of the embodiments described herein for Formula III, K is an ethanolamine moiety (e.g., —CH₂—CH₂—NH—, or —CH₂—CH₂— attached to a nitrogen atom), a serine moiety (e.g., —CH₂—CH(CO₂H)—NH—, or —CH₂—CH(CO₂H)— attached to a nitrogen atom), a glycerol moiety (e.g., —CH(OH)—CH(OH)—CH—O—) and an inositol moiety (e.g., -cyclohexyl(OH)₄—O—). In some embodiments, J is —P(═O)(OH)—O—.

In some embodiments of any of the embodiments described herein for Formula III, M is amido, optionally —C(═O)NH—.

In some embodiments, the nitrogen atom of the amido is attached to K. In some such embodiments, K is an ethanolamine or serine moiety described herein.

In some embodiments of any of the embodiments described herein for Formula III, Q is a substituted alkylene (e.g., of 1 to 6 or 1 to 4 or 1 to 2 carbon atoms in length, for example, a methylene group). In some such embodiments, M is amido or carboxy. In some embodiments, the C(═O) of the amido or the carboxy is attached to Q.

In some embodiments of any of the embodiments described herein for Formula III, Q is an alkylene group as described herein, and the methylene group substituted by one or two substituents, and at least one of these substituents is or comprises an aryl (e.g., phenyl). In some such embodiments, M is amido. In some embodiments, the C(═O) of the amido is attached to Q. Alternatively, M is carboxy and the C(═O) of the carboxy is attached to Q.

In some embodiments of any of the embodiments described herein for Formula III, Q is a methylene group substituted by two substituents, at least one being or comprising an aryl (e.g. phenyl), and the other can be, for example, an aryl (e.g., phenyl) or alkyl (e.g., of 1 to 6 or 1 to 4, carbon atoms in length). In some embodiments, the methylene group is substituted by an alkyl groups (e.g., C₁₋₄-alkyl) and an aryl (e.g., phenyl). In some such embodiments, M is amido. In some such embodiments, M is carboxy.

In some embodiments of any of the embodiments described herein for Formula III, Q is a substituted alkylene (e.g., of 1 to 6 or 1 to 4 or 1 to 2 carbon atoms in length, for example, a methylene group). In some such embodiments, M is amido or carboxy. In some embodiments, the C(═O) of the amido or the carboxy is attached to Q.

In some embodiments of any of the embodiments described herein for Formula III, Q is an alkylene group as described herein, and the methylene group substituted by one or two substituents. In some embodiments, the methylene group is substituted by one or two alkyl groups (e.g., C₁₋₄-alkyl). In some such embodiments, M is other than amido. In some such embodiments, M is carboxy.

In some embodiments of any of the embodiments described herein for Formula III, Q is a methylene group substituted by two substituents. In some embodiments, the methylene group is substituted by two alkyl groups (e.g., C₁₋₄-alkyl). In some embodiments, the alkyl groups are methyl, such that Q is dimethylmethylene (—C(CH₃)₂—). In some such embodiments, M is other than amido. In some such embodiments, M is carboxy.

According to some of any of the embodiments described herein for Formula III, when M is amido, Q is an alkylene that is substituted by at least one aryl as described herein in any of the respective embodiments.

According to some of any of the embodiments described herein, M is other than amido, and Q is as described herein in any of the respective embodiments.

In some embodiments of any of the embodiments described herein for Formula III, M and Q are each absent, and K is terminated by a substituted or unsubstituted methylene group, according to any of the respective embodiments described herein with respect to Q, for example, a methylene group substituted by two substituents (e.g., dimethylmethylene (—C(CH₃)₂—)). In some embodiments, K further comprises a carbonyl group according to any of the respective embodiments described herein.

In some embodiments of any of the embodiments described herein, J, M and Q are each absent. In some such embodiments, K comprises a carbonyl linking group (—C(═O)—) attached directly to the depicted oxygen atom of a glycerol moiety (via an ester bond), and further comprises a substituted or unsubstituted methylene group (e.g., dimethylmethylene). In some embodiment, K consists of a carbonyl linking group attached directly to the depicted oxygen atom of a glycerol moiety (via an ester bond), and a substituted or unsubstituted methylene group, for example, K is —C(═O)—C(CH₃)₂—.

According to some of any of the embodiments described herein for Formula IV, F₁ is as described herein for OW₁. According to some of these embodiments, F₃ and F₄ are each hydrogen.

According to some of these embodiments, J is absent. According to some of these embodiments, M is other than amido.

According to some of any of the embodiments described herein for Formula IV, F₂ is as described herein for OW₂. According to some of these embodiments, F₃ and F₄ are each hydrogen.

According to some of these embodiments, J is absent. According to some of these embodiments, M is other than amido.

According to some of any of the embodiments described herein for Formula IV, F₁ is as described herein for OW₁ and F₂ is as described herein for OW₂. According to some of these embodiments, F₃ and F₄ are each hydrogen. According to some of these embodiments, J is absent.

According to some of these embodiments, M is other than amido.

According to some of any of the embodiments described herein, the lipid moiety does not include a moiety of Formula III as described herein.

According to some of any of the embodiments described herein, the lipid moiety has a moiety of Formula III as described herein, provided that M is other than amido (e.g., M is carboxy) and/or that Q comprises an aryl substituent, as described herein.

According to some of any of the embodiments described herein for Formula IV, excluded from the scope of the present embodiments are polymeric compounds in which the lipid moiety X is as described in WO 2017/109784.

### Targeting Moiety:

As described hereinabove, in some embodiments of any of the embodiments described herein, at least a portion of the monomeric units comprise a targeting moiety (according to any of the embodiments described herein relating to a targeting moiety).

Herein, a “targeting moiety” refers to a moiety which is capable of bringing a compound (e.g., a compound according to some embodiments of the invention) into proximity with a selected substance and/or material (which is referred to herein as a “target”). The target is optionally a cell (e.g., a proliferating cell associated with the proliferative disease or disorder), wherein the proximity is such that the targeting moiety facilitates attachment and/or internalization of the compound into a target cell, and such that the compound may exert a therapeutic effect.

In any of the embodiments described herein wherein m is at least 1, at least a portion of the monomeric units comprising a targeting moiety (the number of which is represented by the variable m), according to any of the respective embodiments described herein, are monomeric units which do not comprise the pendant group represented by (-L-Z). In some such embodiments, each of the monomeric units comprising a targeting moiety (according to any of the respective embodiments described herein) is a monomeric unit which comprises the pendant group represented by (-L-Z) (i.e., a backbone unit Y substituted by (-L-Z)), that is, none of the monomeric units comprising the pendant group represented by (-L-Z) comprise the aforementioned targeting moiety.

In any of the embodiments described herein wherein m is at least 1, each of the monomeric units which do not comprise the pendant group represented by (-L-Z) (the number of which is represented by the variable m) comprises a targeting moiety (according to any of the respective embodiments described herein). In some such embodiments, each of the monomeric units comprising a targeting moiety (according to any of the respective embodiments described herein) is a monomeric unit which does not comprise the pendant group represented by (-L-Z), that is, none of the monomeric units comprising the pendant group represented by (-L-Z) comprise the aforementioned targeting moiety, and each of the monomeric units which does not comprise the pendant group represented by (-L-Z) comprises the aforementioned targeting moiety.

In any of the embodiments described herein wherein m is at least 1, a monomeric unit comprising a targeting moiety may consist essentially of a backbone unit Y (according to any of the respective embodiments described herein) substituted by one or more targeting moieties (according to any of the respective embodiments described herein).

The backbone unit Y of a monomeric unit comprising a targeting moiety may optionally be different (optionally considerably different) in structure than a backbone unit Y of other monomeric units in the polymeric moiety (according to any of the respective embodiments described herein).

In any of the embodiments described herein wherein m is at least 1, the polymeric moiety comprises a monomeric unit which comprises a targeting moiety, and said monomeric unit is at a terminus of the polymeric moiety distal to the lipid moiety. In such embodiments, the compound represented by general formula I has the Formula Ib:

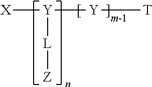

wherein:
T is a monomeric unit comprising a targeting moiety (according to any of the respective embodiments described herein);
X and T are attached to distal termini of the moiety represented by [Y(-L-Z)]n[Y]m−1; and
X, Y, L, Z, n and m are defined in accordance with any of the embodiments described herein relating to Formula I, with the proviso that m is at least 1.

It is to be understood that T in Formula Ib is a type of monomeric unit represented by Y (i.e., without the pendant group represented by (-L-Z)) in formulas I and Ia, and the number of monomeric units represented by Y (i.e., without the pendant group represented by (-L-Z)) other than T is represented by the value m−1, such that the total number of monomeric units without the pendant group represented by (-L-Z)), including T, is represented by the variable m, as in formulas I and Ia.

In some embodiments, m is 1, such that m−1 is zero, and the compound represented by Formula Ib consequently has the formula: X—[Y(-L-Z)]n-T, wherein L, T, X, Y, Z and n are defined in accordance with any of the embodiments described herein.

A monomeric unit comprising a targeting moiety according to any of the respective embodiments described herein may optionally be prepared by preparing a monomer comprising a targeting moiety, and using said monomer to prepare a polymeric moiety described herein (e.g., by polymerization of monomers according to any of the respective embodiments described herein) and/or by modifying a monomeric unit in a polymeric moiety subsequently to preparation of a polymeric moiety (e.g., by polymerization of monomers according to any of the respective embodiments described herein), using any suitable technique known in the art, including, but not limited to, techniques for conjugation.

In some embodiments of any of the embodiments described herein relating to a targeting moiety, the targeting moiety does not comprise a moiety having general formula II (according to any of the respective embodiments described herein). For example, even if a moiety represented by Formula TT is capable of forming a bond with a target as described herein, the phrase “targeting moiety”, in some embodiments, is to be understood as relating to a moiety distinct from a moiety represented by variable Z (having general formula II).

In some embodiments of any one of the embodiments described herein, the pendant group represented by (-L-Z) is selected so as not to form a bond with the target and/or so as not to include a structure and/or property of a targeting moiety as described herein in any one of the respective embodiments. For example, in embodiments wherein a targeting moiety comprising a nucleophilic group (according to any of the respective embodiments described herein)—for example, an amine group—is capable of forming a bond (e.g., covalent bond) with a target, the variable Z (having general formula II) is optionally selected such that the depicted amine/ammonium group is a tertiary amine/ammonium (i.e., no more than one of R₁-R₃ is hydrogen) or quaternary ammonium (i.e., none of R₁-R₃ is hydrogen), preferably a quaternary ammonium (e.g., comprising a trimethylamino group, such as in phosphocholine). Tertiary amine groups, and especially quaternary ammonium groups, may be significantly less reactive nucleophilic groups than primary and secondary amine groups.

In some embodiments of any of the embodiments described herein relating to a targeting moiety, the targeting moiety comprises (and optionally consists of) at least one functional group capable of forming a covalent bond or non-covalent bond (preferably a selective non-covalent bond) with a substance and/or material (which is referred to herein as a “target”), e.g., at a surface of the target (e.g., a surface of a cell and/or tissue).

Herein, the phrase “functional group” encompasses chemical groups and moieties of any size and any functionality described herein (for example, any functionality capable of forming a covalent bond or non-covalent bond with a target).

A non-covalent bond according to any of the respective embodiments described herein may optionally be effected by non-covalent interactions such as, without limitation, electrostatic attraction, hydrophobic bonds, hydrogen bonds, and aromatic interactions.

In some embodiments, the targeting moiety comprises a functional group capable of forming a non-covalent bond which is selective for the target, e.g., an affinity (e.g., as determined based on a dissociation constant) of the targeting moiety and/or functional group to the target is greater than an affinity of the of the targeting moiety and/or functional group to most (or all) other compounds capable of forming a non-covalent bond with the targeting moiety.

In some embodiments of any one of the embodiments described herein, the functional group(s) are capable of forming a covalent bond with one or more specific functional groups (e.g., hydroxy, amine, thiohydroxy and/or oxo groups) which are present on the target (e.g., a target according to any of the respective embodiments described herein).

Examples of functional groups (in a targeting moiety) capable of forming a covalent bond with a target (according to any of the respective embodiments described herein) and the type of covalent bonds they are capable of forming, include, without limitation:
nucleophilic groups such as thiohydroxy, amine (e.g., primary or secondary amine) and hydroxy, which may form covalent bonds with, e.g., a nucleophilic leaving group (e.g., any nucleophilic group described herein), Michael acceptor (e.g., any Michael acceptor described herein), acyl halide, isocyanate and/or isothiocyanate (e.g., as described herein) in a target;
nucleophilic leaving groups such as halo, azide (—N₃), sulfate, phosphate, sulfonyl (e.g. mesyl, tosyl), N-hydroxysuccinimide (NHS) (e.g. NHS esters), sulfo-N-hydroxysuccinimide, and anhydride, which may form covalent bonds with, e.g., a nucleophilic group (e.g., as described herein) in a target;
Michael acceptors such as enones (e.g., maleimide, acrylate, methacrylate, acrylamide, methacrylamide), nitro groups and vinyl sulfone, which may form covalent bonds with, e.g., a nucleophilic group (e.g., as described herein) in a target, optionally thiohydroxy;
dihydroxyphenyl groups (according to any of the respective embodiments described herein), which may form covalent bonds with, e.g., a nucleophilic group (e.g., as described herein) and/or a substituted or unsubstituted phenyl group (e.g., another dihydroxyphenyl group) in a target, as described herein;
an acyl halide (—C(═O)-halogen), isocyanate (—NCO) and isothiocyanate (—N═C═S) group, which may form covalent bonds with, e.g., a nucleophilic group (e.g., as described herein) in a target;
a carboxylate (—C(═O)OH) group, which may form a covalent bond with, e.g., a hydroxyl group in a target to form an ester bond and/or an amine group (e.g., primary amine) in a target to form an amide bond (optionally by reaction with a coupling reagent such as a carbodiimide); and/or a carboxylate group is in a target and may form an amide or ester bond with an amine or hydroxyl group, respectively, in the targeting moiety;
an oxo group (optionally in an aldehyde group (—C(═O)H)), which may form a covalent imine bond with an amine group (e.g., a primary amine) in a target; and/or an oxo group (optionally in an aldehyde group) is in a target and may form a covalent imine bond with an amine groups in the targeting moiety; and/or
thiohydroxy groups, which may form covalent disulfide (—S—S—) bonds with a thiohydroxy group in a target.

Modification of a monomer (e.g., prior to polymerization) or a monomeric unit of a polymeric moiety (e.g., subsequent to polymerization) to comprise any of the functional groups described herein may optionally be performed using any suitable technique for conjugation known in the art. The skilled person will be readily capable of selecting a suitable technique for any given molecule to be modified.

Herein, the term “dihydroxyphenyl” refers to an aryl group (as defined herein) which is a phenyl substituted by two hydroxyl groups at any positions thereof. The phenyl may optionally be substituted by additional substituents (which may optionally comprise additional hydroxyl groups), to thereby form a substituted dihydroxyphenyl group; or alternatively, the phenyl comprises no substituents other than the two hydroxyl groups, such that the dihydroxyphenyl group is an unsubstituted dihydroxyphenyl group.

In some embodiments of any one of the embodiments described herein, the dihydroxyphenyl group is an ortho-dihydroxyphenyl (wherein the hydroxyl groups are attached to the phenyl at adjacent positions) or a para-dihydroxyphenyl (wherein the hydroxyl groups are attached to opposite sides of the phenyl ring), each being a substituted or unsubstituted dihydroxyphenyl. In some such embodiments, the ortho-dihydroxyphenyl or para-dihydroxyphenyl is an unsubstituted dihydroxyphenyl.

A dihydroxyphenyl group according to any of the respective embodiments described herein may optionally bond covalently and/or non-covalently to a target according to any one or more attachment mechanism described for dihydroxyphenyl (catechol) groups in Lee et al. [PNAS 2006, 103:12999-13003], Brodie et al. [Biomedical Materials 2011, 6:015014] and/or International Patent Application PCT/IL2015/050606, the contents of each of which are incorporated in their entirety, and especially contents regarding bonds formed by dihydroxyphenyl (catechol) groups to surfaces.

In some embodiments of any one of the embodiments described herein, the functional group capable of forming a bond to a target is a functional group capable of forming a covalent bond with an amine group, optionally a primary amine group. In some such embodiments, the target comprises on or more amino acids or amino acid residues, for example, a peptide or polypeptide of any length (e.g., at least two amino acid residues, for example, proteins), and the amine groups may optionally be lysine side chain amine groups and/or N-terminal amine groups. In some embodiments, the target comprises an extracellular matrix protein, for example, collagen. In some embodiments, the target comprises cartilage (e.g., articular cartilage).

In some embodiments of any one of the embodiments described herein, the targeting moiety comprises (and optionally consists of) at least one functional group capable of forming a non-covalent bond with the target (e.g., as described herein in any one of the respective embodiments).

In some embodiments of any one of the embodiments described herein, a functional group capable of forming a non-covalent bond with the target comprises (and optionally consists of) a polysaccharide and/or polypeptide (e.g., a protein and/or fragment thereof), wherein the target optionally comprises a ligand of the polysaccharide and/or polypeptide; and/or the target comprises a polysaccharide and/or polypeptide (e.g., a protein and/or fragment thereof) and the functional group capable of forming a non-covalent bond with the target is a ligand of the polysaccharide and/or polypeptide.

Examples of suitable polysaccharides and/or polypeptides, and ligands thereof, include, without limitation:
avidin or streptavidin as a polypeptide described herein, and biotin as a ligand thereof;
a polysaccharide-binding polypeptide as a polypeptide described therein, and a complementary polysaccharide as a ligand thereof (or a complementary polysaccharide-binding polypeptide as a ligand of a polysaccharide described herein);
a collagen-binding polypeptide as a polypeptide described therein, and a complementary collagen as a ligand thereof (or a collagen as a polypeptide described herein and a complementary collagen-binding polypeptide as a ligand thereof);
a cell receptor expressed by a cell, and a ligand selectively bound by the receptor;
an antibody towards any antigen (e.g., wherein the target described herein optionally comprises the antigen) or a fragment of such an antibody as a polypeptide described herein, and the respective antigen as a ligand thereof; and
an antibody mimetic towards any antigen (e.g., wherein the target described herein optionally comprises the antigen).

Examples of cell receptors expressed by a cell include, without limitation, receptors characteristic of a particular type of cell and/or tissue, and receptors overexpressed by a cancer cell. The cell receptor or the cell is optionally a target described herein, and the targeting moiety optionally comprises any ligand of the receptor. Examples of such ligands include, without limitation, transferrin, a ligand of transferrin receptor which may optionally target transferrin receptor overexpressed by some cancer cells; keratinocyte growth factor (KGF or FGF7) which is specific for cells of epithelial origin, and may optionally target KGF receptor such as that overexpressed by an endometrial carcinoma or pancreatic carcinoma [Visco et al., Int J Oncol 1999, 15:431-435; Siegfried et al., Cancer 1997, 79:1166-1171]; and epidermal growth factor (EGF) which may optionally target an EGF receptor, optionally an erbB, such as that overexpressed by gliomas and endometrial carcinomas [Normanno et al., Curr Drug Targets 2005, 6:243-257]).

As used herein, the term “antibody” encompasses any type of immunoglobin.

As used herein, the phrase “antibody mimetic” encompasses any type of molecule, optionally a polypeptide, referred as such in the art capable of selectively binding an antigen (e.g., non-covalently). Non-limiting examples of antibody mimetics include affibodies, affilins, affimers, affitins, alphabodies, anticalins, avimers, DARPins, Fynomers, Kunitz domain peptides, and monobodies, e.g., as described in Nygren [FEBS J 2008, 275:2668-2676], Ebersbach et al. [J Mol Biol 2007, 372:172-185], Johnson et al. [Anal Chem 2012, 84:6553-6560], Krehenbrink et al. [J Mol Biol 2008, 383:1058-1068], Desmet et al. [Nature Comm 2014, 5:5237], Skerra [FEBS J 2008, 275:2677-2683], Silverman et al. [Nature Biotechnol 2005, 23:1556-1561], Stumpp et al. [Drug Discov Today 2008, 13:695-701], Grabulovski et al. [J Biol Chem 2007, 282:3196-3204], Nixon & Wood [Curr Opin Drug Discov Devel 2006, 9:261-268], Koide & Koide [Methods Mol Biol 2007, 325:95-109], and Gebauer & Skerra [Curr Opin Chem Biol 2009, 13:245-255], the contents of each of which are incorporated in their entirety, and especially contents regarding particular types of antibody mimetics.

As used herein, the phrase “polysaccharide-binding polypeptide” encompasses any polypeptide or oligopeptide (peptide chains of at least 2, and preferably at least 4 amino acid residues in length) capable of selectively binding (e.g., non-covalently) to a polysaccharide. A wide variety of polysaccharide-binding polypeptides and their binding specificities will be known to the skilled person, and include short peptide sequences (e.g., from 4 to 50, optionally 4 to 20 amino acid residues in length), and longer polypeptides such as proteins or fragments (e.g., carbohydrate-binding modules and/or domains) thereof. In addition, the phrase “polysaccharide-binding polypeptide” encompasses antibodies capable of specifically binding to a polysaccharide. Such antibodies will be available to the skilled person and/or the skilled person will know how to prepare such antibodies, using immunological techniques known in the art.

Examples of polysaccharide-binding polypeptides which may be used in some of any one of the embodiments of the invention include, without limitation, carbohydrate-binding modules (CBMs); and hyaluronic acid-binding peptides, polypeptides and/or modules (e.g., having a sequence as described in any of International Patent Application publication WO 2013/110056; International Patent Application publication WO 2014/071132; Barta et al. [Biochem J 1993, 292:947-949], Kohda et al. [Cell 1996, 86:767-775], Brisset & Perkins [FEBS Lett 1996, 388:211-216], Peach et al. [J Cell Biol 1993, 122:257-264], Singh et al. [Nature Materials 2014, 13:988-995], and Zaleski et al. [Antimicrob Agents Chemother 2006, 50:3856-3860], the contents of each of which are incorporated in their entirety, and especially contents regarding particular polysaccharide-binding polypeptides), for example, GAHWQFNALTVR (a hyaluronic acid-binding peptide sequence).

Examples of CBMs which may be used in some of any one of the embodiments of the invention, include, without limitation, CBMs belonging to the families CBM3, CBM4, CBM9, CBM10, CBM17 and/or CBM28 (which may optionally be used to bind cellulose, e.g., in a cellulose-containing target); CBM5, CBM12, CBM14, CBM18, CBM19 and/or CBM33 (which may optionally be used to bind chitin and/or other polysaccharides comprising N-acetylglucosamine, e.g., in a chitin-containing target); CBM15 (which may optionally be used to bind hemicellulose, e.g., in a hemicellulose-containing target); and/or CBM20, CBM21 and/or CBM48 (which may optionally be used to bind starch and/or glycogen, e.g., in a starch-containing and/or glycogen-containing target).

As used herein, the phrase “collagen-binding polypeptide” encompasses any polypeptide or oligopeptide (peptide chains of at least 2, and preferably at least 4 amino acid residues in length) capable of selectively binding (e.g., non-covalently) to a collagen (e.g., one type of collagen, some types of collagen, all types of collagen), including glycosylated polypeptides and oligopeptides such as peptidoglycans and proteoglycans. A wide variety of collagen-binding polypeptides and their binding specificities will be known to the skilled person, and include short peptide sequences (e.g., from 4 to 50, optionally 4 to 20 amino acid residues in length), and longer polypeptides such as proteins or fragments (e.g., collagen-binding domains) thereof. In addition, the phrase “collagen-binding polypeptide” encompasses antibodies capable of specifically binding to a collagen. Such antibodies will be available to the skilled person and/or the skilled person will know how to prepare such antibodies, using immunological techniques known in the art.

Examples of collagen-binding polypeptides which may be used in embodiments of the invention include, without limitation, collagen-binding proteins (e.g., decorin), fragments thereof and/or other polypeptides as described in U.S. Pat. No. 8,440,618, Abd-Elgaliel & Tung [Biopolymers 2013, 100:167-173], Paderi et al. [Tissue Eng Part A 2009, 15:2991-2999], Rothenfluh et al. [Nat Mater 2008, 7:248-254] and Helms et al. [J Am Chem Soc 2009, 131:11683-11685](the contents of each of which are incorporated in their entirety, and especially contents regarding particular collagen-binding polypeptides), for example, the sequence WYRGRL.

It is expected that during the life of a patent maturing from this application many relevant functional groups and moieties for binding will be developed and/or uncovered and the scope of the terms “targeting moiety”, “functional group”, “cell receptor”, “antibody”, “antibody mimetic”, “collagen-binding polypeptide” and “polysaccharide-binding polypeptide” and the like is intended to include all such new technologies a priori.

In some embodiments of any of the embodiments described herein, a functional group in a targeting moiety (according to any of the respective embodiments described herein) is attached to a linking group (as defined herein). The linking group may optionally be any linking group or linking moiety described herein, including, without limitation, a substituted or unsubstituted hydrocarbon. In some embodiments, the targeting moiety (optionally a substituent of a backbone unit Y) consists essentially of a functional group attached to the rest of the polymeric moiety via the linking group.

A functional group may optionally be attached to the linking moiety by a covalent bond obtainable by a reaction between two functional groups, for example, any covalent bond and/or functional groups described herein in the context of forming a covalent bond between a functional group and a target.

In some embodiments of any of the embodiments described herein relating to a functional group comprising a peptide or polypeptide, an amino acid residue of the peptide or polypeptide is optionally attached to a linking group of the targeting moiety, for example, via an amide bond formed from an amine or carboxylate group in the peptide or polypeptide (e.g., in an N-terminus, a lysine side chain, a C-terminus, a glutamate side chain and/or an aspartate side chain), an ester bond formed from a hydroxyl or carboxylate group in the peptide or polypeptide (e.g., in a serine side chain, a threonine side chain, a C-terminus, a glutamate side chain and/or an aspartate side chain), and/or a disulfide bond formed from a thiohydroxy group in the peptide or polypeptide (e.g., in a cysteine side chain). In some embodiments, an amino acid residue attached to the linking group is an N-terminal and/or C-terminal residue, for example, any amino acid residue attached via an N-terminal amino group or C-terminal carboxylate group, and/or a terminal lysine, glutamate, aspartate, serine, threonine and/or cysteine residue attached via a side chain thereof.

In some embodiments, an amino acid residue and/or peptide (e.g., from 2 to 20 amino acid residues in length) is added to the N-terminus and/or C-terminus of a peptide or polypeptide sequence of a functional group (according to any of the respective embodiments described herein), and links the aforementioned sequence to a linking group. Examples of such terminal amino acid residues and/or peptides include, without limitation, glycine residues and peptides with a terminal glycine residue, which may be used to attach a linking group to an N-terminus or C-terminus (according to any of the respective embodiments described herein); serine and threonine residues and peptides with a terminal serine or threonine residue, which may be used to attach a linking group to hydroxyl group in a serine or threonine side chain, optionally via an ester bond (according to any of the respective embodiments described herein); and cysteine residues and peptides with a terminal cysteine residue, which may be used to attach a linking group to a peptide via a disulfide bond (according to any of the respective embodiments described herein).

In some embodiments, attachment of a peptide or polypeptide to a linking group via a terminal amino acid residue minimizes interference (e.g., steric interference) with the functionality of the peptide or polypeptide following attachment to the linking group.

In some embodiments, attachment of a peptide or polypeptide to a linking group via a terminal glycine facilitates attachment by minimizing interference (e.g., steric interference) of an amino acid side chain (which glycine lacks) with attachment to the linking group.

According to some of any of the embodiments described herein, the polymeric compounds are as described in U.S. Provisional Patent Application No. 63/402,097, by the present assignee, which is incorporated by reference as if fully set forth herein.

### Bilayer-Forming Lipid and Liposomes:

As described herein, the liposome according to embodiments comprises, inter alia, at least one bilayer-forming lipid.

Herein, the term “bilayer-forming lipid” encompasses any compound in which a bilayer may form from a pure aqueous solution of the compound, the bilayer comprising two parallel layers of molecules of the compound (referred to as a “lipid”).

Typically, the bilayer (e.g., in a liposome according to some of any of the embodiments described herein) comprises relatively polar moieties of the lipid at the two surfaces of the bilayer, which may optionally comprise an interface with the aqueous solution and/or an interface with a solid surface; and relatively hydrophobic moieties of the lipid at the interior of the bilayer, at an interface between the two layers of lipid molecules which form the bilayer.

Examples of bilayer-forming lipids include glycerophospholipids. Suitable examples of glycerophospholipids include, without limitation, a phosphatidyl ethanolamine, a phosphatidyl serine, a phosphatidyl glycerol and a phosphatidyl inositol.

It is to be appreciated that the polymeric compound comprised by a liposome (according to any of the respective embodiments described herein) may optionally be a bilayer-forming lipid which can form a bilayer per se or in combination with one or more additional bilayer-forming lipids.

In some embodiments of any one of the embodiments described herein, the bilayer-forming lipid comprises at least one charged group (e.g., one or more negatively charged groups and/or one or more positively charged groups).

In some embodiments, the bilayer-forming lipid is zwitterionic; comprising both (e.g., an equal number of) negatively charged and positively charged groups (e.g., one of each).

In some embodiments of any of the embodiments described herein, a mol ratio of the bilayer-forming lipid (comprised in addition to the polymeric compound) and the polymeric compound (according to any of the respective embodiments described herein) in the liposome is in a range of from 5:1 to 5,000:1 (bilayer-forming lipid: polymeric compound), optionally in a range of from 10:1 to 2,500:1, optionally in a range of from 25:1 to 1,000:1, and optionally in a range of from 50:1 to 500:1, including any intermediate values and subranges therebetween.

In some embodiments of any of the embodiments described herein relating to a bilayer, a mol ratio of the bilayer-forming lipid (comprised in addition to the polymeric compound) and the polymeric compound in the bilayer is in a range of from 10:1 to 1,000:1 (bilayer-forming lipid: polymeric compound), optionally in a range of from 10:1 to 500:1, optionally in a range of from 10:1 to 200:1, and optionally in a range of from 10:1 to 150:1, including any intermediate values and subranges therebetween.

In some embodiments of any of the embodiments described herein relating to a bilayer, a mol ratio of the bilayer-forming lipid (comprised in addition to the polymeric compound) and the polymeric compound in the bilayer is in a range of from 10:1 to 100:1 (bilayer-forming lipid: polymeric compound), optionally in a range of from 10:1 to 50:1, optionally in a range of from 20:1 to 40:1, including any intermediate values and subranges therebetween.

In some embodiments of any of the embodiments described herein relating to a bilayer, a mol ratio of the bilayer-forming lipid (comprised in addition to the polymeric compound) and the polymeric compound in the bilayer is in a range of from 10:1 to 1,000:1 (bilayer-forming lipid: polymeric compound), optionally in a range of from 100:1 to 1,00:1, optionally in a range of from 101:1 to 500:1, and optionally in a range of from 100:1 to 200:1, including any intermediate values and subranges therebetween.

Herein throughout, the terms “mol ratio” and “molar ratio” are used interchangeably, and describe the ratio between the mol % of the indicated components in the lipid bilayer or liposome.

The bilayer according to embodiments described herein may optionally be closed upon itself (e.g., such that the bilayer has no edges), thereby forming an inner volume separated by the bilayer from the surrounding environment, which is referred to herein and in the art as a “liposome”. Alternatively or additionally, the bilayer may be open-faced and/or with edges.

As used herein and in the art, the term “liposome” refers to an artificially prepared vesicle comprising a bilayer composed of molecules of an amphiphilic lipid. In an aqueous medium, the bilayer is typically configured such that hydrophilic moieties of the amphiphilic lipid are exposed to the medium at both surfaces of the bilayer, whereas lipophilic moieties of the lipid are located in the internal portion of the bilayer, and therefore less exposed to the medium. Examples of liposomes which may be used in any one of the embodiments described herein include, without limitation, small unilamellar vesicles (SUV), large unilamellar vesicles (LUV) and large multilamellar vesicles (MLV).

As described herein, the liposome according to embodiments comprises, inter alia, at least one bilayer-forming lipid.

It is to be appreciated that the polymeric compound comprised by a liposome (according to any of the respective embodiments described herein) may optionally be a bilayer-forming lipid which can form a bilayer per se or in combination with one or more additional bilayer-forming lipids.

A liposome may optionally comprise a single bilayer (e.g., a unilamellar vesicle) or a plurality of bilayers (e.g., a multilamellar vesicle)—wherein each bilayer optionally independently forms a closed vesicle—comprising, for example, concentric bilayer vesicles and/or a plurality of separate bilayer vesicles encompassed by the same bilayer vesicle.

As used herein, the term “unilamellar” refers to liposomes characterized by a single lipid bilayer, whereas the term “multilamellar” refers to liposomes characterized by a multiple lipid bilayers, for example, concentric bilayers.

As used herein, the phrase “small unilamellar vesicle” refers to unilamellar liposomes of less than 100 nm in diameter, whereas the phrase “large unilamellar vesicle” refers to unilamellar liposomes at least 100 nm in diameter.

In some embodiments of any one of the embodiments described herein, the liposomes comprise multilamellar vesicles. In some embodiments, the liposomes are primarily (more than 50 weight percent) multilamellar vesicles.

In some embodiments, the liposomes are primarily (more than 50 weight percent) large multilamellar vesicles, at least 100 nm in diameter (MLV).

A liposome according to any of the respective embodiments described herein may be approximately spherical in shape or may have any alternative shape, such as an elongated tube and/or a flattened (e.g., sheet-like) shape.

In some of any of the embodiments described herein, a weight ratio of the bilayer-forming lipid and the polymeric compound is in a range of from 2:1 to 1,000:1, or from 2:1 to 500:1, or from 2:1 to 100:1, or from 2:1 to 50:1 or from 2:1 to 20:1.

In some of any of the embodiments described herein, a mean diameter of liposomes ranges from about 50 nm to about 5,000 nm, or from about 500 nm to about 5,000 nm, or from about 500 nm to about 4,000 nm, or from about 500 nm to about 3500 nm, including any intermediate values and subranges therebetween.

In some of any of the embodiments described herein, a mean diameter of liposomes ranges from about 100 nm to about 2000 nm, or from about 100 nm to about 1000 nm, or from 200 nm to 2,000 nm, or from 200 nm to 1,000 nm, from about 500 nm to about 1000 nm, or from 400 to about 800 nm, including any intermediate values and subranges therebetween.

The mean diameter according to any of the respective embodiments described herein may optionally be an arithmetic mean (a ratio of a sum of values to the number of values) or a Z-average as this term is defined in the art of dynamic light scattering (in brief, an intensity-weighted harmonic mean). In exemplary embodiments, the mean diameter is a Z-average diameter determined by dynamic light scattering.

The polydispersity index (PDI) and/or mean diameter of liposomes in a composition may optionally be determined by dynamic light scattering using a two-parameter fit to the data (e.g., according to ISO 13321 and ISO 22412 standards) for determining PDI and Z-average diameter (e.g., using a commercially available instrument).

In some of any of the embodiments described herein, a PDI of the liposomes is lower than 1 or lower than 0.8, for example, in a range of from 0.3 to 1, or from 0.3 to 0.8, or from 0.3 to 0.6, including any intermediate values and subranges therebetween.

In some of any of the embodiments described herein, a zeta potential of the liposomes is at least −3 mV (i.e., −3 mV or a more negative value), optionally at least −3.5 mV, and optionally at least −4 mV.

In some of any of the embodiments described herein, a zeta potential of the liposomes is in a range of from 10 mV to −10 mV (e.g., from 5 mV to −5 mV), optionally in a range of from 0 to −10 mV (e.g., from 0 to −5 mV, or from −3 mV to −5 mV).

Zeta potential may optionally be determined using any suitable technique known in the art (e.g., using a commercially available instrument), for example, electrophoretic light scattering.

The zeta potential of liposomes may be determined by diluting the liposomes in an aqueous salt (e.g., NaCl) solution with a predetermined salt concentration (e.g., 10 PM).

In some embodiments of any of the embodiments described herein relating to a liposome, the liposome further comprises at least one functional moiety or agent bound to a surface of the liposome and/or within a lipid bilayer and/or core of the liposome (e.g., within the liposome bilayer and/or enveloped by the liposome bilayer).

Examples of functional moieties and agents suitable for inclusion in embodiments described herein include, without limitation, a therapeutically active agent or moiety of a therapeutically active agent (e.g., wherein the active agent is releasable upon cleavage of the moiety), a labeling moiety or agent, and/or a targeting moiety or targeting agent (e.g., a targeting moiety or agent on a surface of the liposome).

Examples of therapeutically active agents suitable for inclusion in a liposome (e.g., as a molecule or moiety of the agent) include, without limitation, amphotericin B, cisplatin, cytarabine, daunorubicin, doxorubicin, estradiol, influenza virosome, morphine, surfactant protein B, surfactant protein C, verteporfin and vincristine.

In some of any of the embodiments described herein, the liposomes comprise a therapeutically active agent, which is optionally incorporated in a liposome and/or on a surface of the liposome. In some such embodiments, the therapeutically active agent is a therapeutically active agent described in International Patent Application publication WO 2018/150429, which is incorporated herein by reference.

In some of any of the embodiments described herein, the liposomes are devoid of a therapeutically active agent.

Herein, the phrase “therapeutically active agent” refers to any agent (e.g., compounds) having a therapeutic effect, provided that the compound is not a bilayer-forming lipid or polymeric compound comprised by the liposome (according to any of the respective embodiments described herein), as well as to any portion of an agent (e.g., a moiety of a compound) which generates an agent having a therapeutic effect upon release (e.g., upon cleavage of one or more covalent bonds), including a portion of a bilayer-forming lipid or polymeric compound. Thus, the bilayer-forming lipid and polymeric compound per se are excluded from the definition of a therapeutically active agent, but a bilayer-forming lipid and/or polymeric compound may optionally comprise generate a therapeutically active agent upon release, in which case the portion of the bilayer-forming lipid and/or polymeric compound which generates the therapeutically active agent is also considered to be a therapeutically active agent as defined herein.

When associated with a liposome, a therapeutically active agent may optionally be attached by a covalent or non-covalent (e.g., electrostatic and/or hydrophobic) bond to a liposome (e.g., to an exterior surface and/or interior surface of a liposome membrane), incorporated within a liposome membrane (e.g., a lipophilic agent which stably partitions to a lipid phase of the liposome), and/or enveloped within a core of a liposome (e.g., a hydrophilic agent in an aqueous compartment of the liposome). The therapeutically active agent may optionally be a moiety covalently attached to a liposome (e.g., attached to a lipid so as to form a lipid-derivative comprising the moiety). Such attachment may be obtained in some embodiments by using techniques known in the art (e.g., amide bond formation).

According to some of any of the embodiments described herein, the composition comprises comprising a plurality of liposomes, and at least a portion of the liposomes comprises a plurality of liposomes as defined herein. Optionally, the composition comprises an additional portion of liposomes other than the liposomes described herein (e.g., not including a polymeric compound as described herein).

Examples of a labeling moiety or agent include moieties and compounds which are chromophoric (e.g., absorb visible light), fluorescent, phosphorescent, and/or radioactive. Many such compounds and moieties (and techniques for preparing such moieties) will be known to a skilled person.

A targeting moiety in a liposome according to any of the respective embodiments described herein may optionally be a targeting moiety according to any of the respective embodiments described herein. A targeting moiety in a liposome may be comprised by a polymeric compound according to some embodiments of the invention (according to any of the respective embodiments described herein), the liposome comprising the polymeric compound. Alternatively or additionally, a targeting moiety in a liposome may optionally be comprised by another compound in the liposome, optionally a bilayer-forming lipid (according to any of the respective embodiments described herein) conjugated to a targeting moiety according to any of the respective embodiments described herein.

Herein, a “targeting agent” refers to a compound (“agent”) comprising (and optionally consisting essentially of) a targeting moiety according to any of the respective embodiments described herein (e.g., in the context of a targeting moiety comprised by a polymeric compound described herein). Typically, the phrase “targeting agent” is used to refer to a compound other than a polymeric compound comprising a targeting moiety, as described herein.

In some embodiments, a functional moiety (e.g., targeting moiety or labeling moiety) is covalently attached to a liposome. Such attachment may be obtained in some embodiments by using techniques known in the art (e.g., amide bond formation).

### Composition and Uses:

It should be noted that the liposomes disclosed herein being part of a cosmetic composition may be regarded as an active ingredient (e.g. moisturizer, skin protectant), modulator of skin penetration and/or a carrier of other formulation components.

A cosmetic or cosmeceutical composition (also referred to herein interchangeably as formulation) according to the present embodiments can be used as a cosmetic or cosmeceutical product (e.g., skin-care product) per se or be used to make up such a product.

In some embodiment of the present invention, the cosmetic or cosmeceutical formulation is formulated in a form suitable for topical application on the applied area (e.g., a keratinous tissue, for example, facial skin).

By selecting the appropriate carrier and optionally other ingredients that can be included in the formulation, as is detailed hereinbelow, the compositions of the present embodiments may be formulated into any form typically employed for topical application.

By “appropriate carrier” for topical application it is meant any medium compatible with a keratinous substrate or a mucosal tissue, which has a color, a smell and a pleasant feel and which does not generate unacceptable discomfort (stinging, tautness or redness).

According to some of any of the embodiments of these aspects of the present invention, the formulation further comprises a cosmetically or cosmeceutically acceptable carrier.

Herein, the term “cosmetically or cosmeceutically acceptable carrier” refers to a carrier or a diluent that does not cause significant irritation to a keratinous material or tissue of an organism and does not abrogate the biological activity and properties of the applied compound or combinations of compounds (e.g., a liposome as described herein). Examples, without limitations, of carriers include propylene glycol, water, saline, emulsions and mixtures of organic solvents with water, as well as solid (e.g., powdered) and gaseous carriers. The carrier is typically aimed at facilitating the topical application of the composition to the keratinous tissue or substrate. It is to be noted that the carrier is selected in accordance with the intended use and the form of the formulation or the product containing same and is not limited to the above description.

According to some of any of the embodiments described herein the carrier comprises an aqueous liquid, e.g., water or an aqueous solution. According to some embodiments, the liposome is included in the aqueous liquid and the liposome-containing aqueous liquid in incorporated in the composition.

The cosmetic or cosmeceutical formulations as described herein in any of the respective embodiments and any combination thereof can be packaged in a packaging material and identified in print, in or on the packaging material, for use in the treatment of a medical or cosmetic condition, as described in further detail hereinbelow. The package can be adopted for topical application of the formulation, in accordance with its consistency. For example, liquid formulations (including emulsions and oils) can be packaged in a container having means to topically apply the formulation, for example, a nozzle, a dropper, a pipette, or in a narrow-necked and/or squeezable bottle. Formulations in a form of a spray or aerosol can be packaged in a container equipped with a spraying nozzle and/or mechanism, Ointments, gels, oils and creams can be packaged in a simple container, or in a squeezable container.

According to an aspect of some embodiments of the present invention there is provided an article-of-manufacturing, which comprises any of the formulations and/or products as described herein, and optionally means for topically applying the formulations (e.g., as described herein).

“Topical application” means to apply or spread the formulation, composition or product of the present embodiments onto the surface of the keratinous tissue.

In some embodiments, a formulation as described is in a form of a cream, an ointment, a paste, a gel, a lotion, a milk, a suspension, a solution, an aerosol, a spray, a foam, a serum, or a mousse.

The formulation can be water-based, or emulsion-based (including water-in-oil, oil-in-water, water-in-oil-in-water and oil-in-water-in-oil emulsions) or silicon-based.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emolliency). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the sunscreens-containing capsules, are present in a water or alcohol base. Lotions are typically preferred for covering/protecting large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol and/or natural oil substances and/or plant extracts. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the skin of a user, quickly evaporates, driving the active ingredient through the upper skin layers to the site of treatment.

According to specific embodiments, the composition may comprise an aqueous phase.

The aqueous phase of a composition according to the invention comprises water and optionally a water-soluble solvent.

In the present invention, the term “water-soluble solvent” denotes a compound that is liquid at room temperature and water-miscible (miscibility with water of greater than 50% by weight at 25 °C and atmospheric pressure).

The water-soluble solvents that may be used in the composition of the invention may also be volatile.

Among the water-soluble solvents that may be used in the composition in accordance with the invention, mention may be made especially of lower monoalcohols containing from 2 to 8 carbon atoms, such as ethanol and isopropanol, glycols containing from 2 to 8 carbon atoms, such as ethylene glycol, propylene glycol, 1,3-butylene glycol and dipropylene glycol, C3 and C4 ketones and C2-C4 aldehydes.

The aqueous phase (water and optionally the water-miscible solvent) may be present in the composition in a content ranging from 20% to 95%, better still from 30% to 80% by weight by weight relative to the total weight of the said composition.

According to another embodiment variant, the aqueous phase of a composition according to the invention may comprise at least one C2-C32 polyol.

For the purposes of the present invention, the term “polyol” should be understood as meaning any organic molecule comprising at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols advantageously suitable for the formulation of a composition according to the present invention are those exhibiting in particular from 2 to 32 carbon atoms and preferably from 3 to 16 carbon atoms.

Advantageously, the polyol may be chosen, for example, from ethylene glycol, pentaerythritol, trimethylolpropane, propylene glycol, 1,3-propanediol, butylene glycol, isoprene glycol, pentylene glycol, hexylene glycol, glycerol, polyglycerols such as glycerol oligomers, for instance diglycerol, and polyethylene glycols, and mixtures thereof.

According to a preferred embodiment of the invention, the said polyol is chosen from ethylene glycol, pentaerythritol, trimethylolpropane, propylene glycol, glycerol, polyglycerols and polyethylene glycols, and mixtures thereof.

According to a particular embodiment, the composition of the invention may comprise at least propylene glycol.

According to another particular embodiment, the composition of the invention may comprise at least glycerol.

A water suitable for the invention can be a floral water such as cornflower water and/or a mineral water such as water from Vittel, water from Lucas or water from La Roche Posay, and/or thermal water.

According to specific embodiments, the composition comprises alcohol.

According to a particular form of the invention, the composition contains at least one mono-alcohol comprising from 2 to 8 carbon atoms.

According to a particular form of the invention, the composition contains from 0.5% to 10%, and preferably from 1 to 5 by weight relative to the total weight of at least one mono-alcohol comprising from 2 to 8 carbon atoms.

The compositions of the invention comprise at least one mono-alcohol having from 2 to 8 carbon atoms, especially from 2 to 6 carbon atoms, and particularly 2 to 4 carbon atoms.

The compositions of the invention may include one or more mono-alcohol (s).

The monoalcohol may be represented for example by formula RaOH, wherein Ra is an alkyl group, linear or branched, comprising 2 to 8 carbon atoms.

As a monohydric alcohol include ethanol, isopropanol, propanol or butanol.

According to one embodiment, the compositions of the invention include ethanol.

In any of the formulations or products described herein, additional agents and/or additives can be included.

Some non-limiting representative examples of additives and/or agents include humectants, antioxidants, solvents, odor absorbers, perfumes, deodorants, antiperspirants, sunscreen agents (e.g., UV blocking agents, UV filters), sunless tanning agents, hair conditioning agents, pH adjusting agents, chelating agents, preservatives, emulsifiers, occlusive agents, emollients, thickeners, solubilizing agents, penetration enhancers, anti-irritants, colorants or coloring agents (pigments, nacres, water-soluble dyestuffs), propellants, surfactants, dispersants, fillers and bactericides.

Representative examples of humectants include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propyleneglycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Suitable pH adjusting agents include, for example, one or more of adipic acids, glycines, citric acids, calcium hydroxides, magnesium aluminometasilicates, buffers or any combinations thereof.

Representative examples of deodorant agents include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmithyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate.

Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Representative examples of sunless tanning agents include, without limitation, dihydroxyacetone, glyceraldehyde, indoles and their derivatives. The sunless tanning agents can be used in combination with the sunscreen agents.

The chelating agents are optionally added to formulations so as to enhance the preservative or preservative system. Preferred chelating agents are mild agents, such as, for example, ethylenediaminetetraacetic acid (EDTA), EDTA derivatives, or any combination thereof.

Suitable preservatives include, without limitation, one or more alkanols, disodium EDTA (ethylenediamine tetraacetate), EDTA salts, EDTA fatty acid conjugates, isothiazolinone, parabens such as methylparaben and propylparaben, propyleneglycols, sorbates, urea derivatives such as diazolindinyl urea, or any combinations thereof.

Suitable emulsifiers include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients include, for example, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

According to a particular mode of the invention, the composition contains at least one particulate or non-particulate, water-soluble or water-insoluble coloring agent.

According to specific embodiments, the coloring agent is in a proportion of at least 0.01% by weight relative to the total weight of the composition.

For the purposes of the invention, the term “water-soluble coloring agent” means any natural or synthetic, generally organic compound, which is soluble in an aqueous phase or in water-miscible solvents, and which is capable of imparting colour.

Examples of water-soluble dyes may be made especially of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophylline, methylene blue, anthocyanins (enocianin, black carrot, hibiscus and elder), caramel and riboflavin.

According to specific embodiments, the coloring agent may be pigments, nacres and/or particles with metallic tints.

The term “pigments” should be understood as meaning white or coloured, mineral or organic particles that are insoluble in an aqueous solution, which are intended to color and/or opacity the composition containing them.

The pigments may be white or colored, and mineral and/or organic.

Mineral pigments that may be used with specific embodiments of the invention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30. They may also be pigments having a structure that may be, for example, of silica microsphere type containing iron oxide. An example of a pigment having this structure is the product sold by the company Miyoshi under the reference PC Ball PC-LL-100 P, this pigment being constituted of silica microspheres containing yellow iron oxide.

The term “nacres” should be understood as meaning iridescent or non-iridescent colored particles of any shape, especially produced by certain molluscs in their shell or alternatively synthesized, which have a color effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the nacres available on the market, mention may be made of the nacres Timica, Flamenco and Duochrome (based on mica) sold by the company Engelhard, the Timiron nacres sold by the company Merck, the Prestige mica-based nacres sold by the company Eckart, and the Sunshine synthetic mica-based nacres sold by the company Sun Chemical.

The nacres may more particularly have a yellow, pink, red, bronze, orangey, brown, gold and/or coppery color or tint.

According to a particular mode of the invention, the composition contain at least one filler.

For the purposes of the present invention, the term “fillers” should be understood as meaning colorless or white solid particles of any form, which are in an insoluble and dispersed form in the medium of the composition.

These fillers, of mineral or organic, natural or synthetic nature, give the composition containing them softness and give the makeup result a matt effect and uniformity.

The fillers according to specific embodiments of the present invention may be in lamellar form (or platelet), spherical (or globular), fiber or any other intermediate form between these defined forms.

Non-limiting examples of organic spherical fillers include for example polyamide powders and especially Nylon® powders such as Nylon-12 or Polyamide 12, sold under the names ORGASOL by Arkema; polyethylene powders; polytetrafluoroethylene powders (Teflon *); microspheres based on acrylic copolymers, such as copolymer of ethylene glycol dimethacrylate/lauryl methacrylate copolymer sold by Dow Corning under the name Polytrap; expanded powders such as hollow microspheres and especially the microspheres sold under the name Expancel by Kemanord Plast or under the name Micropearl F 80 ED by Matsumoto; silicone resin microbeads such as those sold under the name Tospearl by Toshiba Silicone; polymethyl methacrylate microspheres, sold under the name Microsphere M-100 by Matsumoto or under the name Covabead LH85 by Wacker; ethylene acrylate copolymer powders, such as those sold under the name Flobeads by Sumitomo Seika Chemicals; powders of natural organic materials such as starch powders, especially of corn starch, wheat or rice, crosslinked or otherwise, such as the powders of starch crosslinked with octenyl succinate anhydride, sold under the name Dry-FLO by National Starch; metal soaps derived from organic carboxylic acids having 8 to 22 carbon atoms, preferably from 12 to 18 carbon atoms, for example, zinc stearate, magnesium or lithium, zinc laurate, myristate magnesium, Polyporus the L*200 (Chemdal Corporation), polyurethane powders, in particular, powders of crosslinked polyurethane comprising a copolymer, said copolymer comprising trimethylol hexyl lactone as the polymer of hexamethylene diisocyanate/trimethylol hexyl lactone, sold under the name Plastic Powder D-400® or Plastic Powder D-800® by the company Toshiki, carnauba microwaxes, such as that sold under the name MicroCare 350® by the company Micro Powders, microwaxes of synthetic wax such as that sold under the name MicroEase 114S® by the company Micro Powders, microwaxes consisting of a mixture of carnauba wax and polyethylene wax, such as those sold under the names Micro Care 300® and 310® by the company Micro Powders, microwaxes consisting of a mixture of carnauba wax and of synthetic wax, such as that sold under the name Micro Care 325® by the company Micro Powders, polyethylene microwaxes such as those sold under the names Micropoly 200®, 220®, and 220L® 250S® by the company Micro Powders. As spherical inorganic filler, there may be mentioned the hydrophobic aerogel silica particles.

Non-limiting examples of lamellar fillers that may be used with specific embodiments of the invention include phyllosilicates, such as talcs, micas, perlite and mixtures thereof.

According to specific embodiments, the composition comprises e a dispersant.

Such a dispersant may be, for example, a surfactant, an oligomer, a polymer or a mixture of several thereof.

Depending on the fluidity of the composition that it is desired to obtain, it is possible to incorporate one or more thickeners or gelling agents into a composition of the invention.

A thickener or gelling agent that is suitable for use in the invention may be hydrophilic, i.e. soluble or dispersible in water.

Hydrophilic gelling or thickening agents that may be mentioned in particular include water-soluble or water-dispersible thickening polymers.

Suitable thickeners include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, fatty acids and their alkali salts and mixtures thereof.

Other non-limiting examples of thickeners may be chosen especially from: modified or unmodified carboxyvinyl polymers, such as the products sold under the name Carbopol (CTFA name: Carbomer) by the company Goodrich; polyacrylates and polymethacrylates such as the products sold under the names Lubrajel and Norgel by the company Guardian or under the name Hispagel by the company Hispano Chimica; polyacrylamides; optionally crosslinked and/or neutralized 2-acrylamido-2-methylpropanesulfonic acid polymers and copolymers, for instance the poly(2-acrylamido-2-methylpropanesulfonic acid) sold by the company Clariant under the name Hostacerin AMPS® (CTFA name: ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company SEPPIC; hydrophobic modified polymers of this type, of the copolymer of ammonium salt of 2-acrylamido-2-methylpropanesulphonic acid and of ethoxylated C12-C14 alkyl methacrylate (noncrosslinked copolymer obtained from ® Genapol LA-070 and from ® AMPS) (CTFA name: Ammonium Acryloyldimethyltaurate/Laureth-7 Methacrylate Copolymer) sold under the name ® Aristoflex LNC by Clariant, and the crosslinked copolymer of ammonium salt of 2-acrylamido-2-methylpropanesulphonic acid and of ethoxylated (25 EO) stearyl methacrylate (copolymer which is preferably crosslinked with trimethylolpropane triacrylate and obtained from Genapol T-250 and from @AMPS) (CTFA name: Ammonium Acryloyldimethyltaurate/Steareth-25 Methacrylate Crosspolymer) sold under the name ® Aristoflex HMS by Clariant; polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethyl cellulose, hydroxymethyl cellulose and hydroxypropyl cellulose; and mixtures thereof.

Representative examples of solubilizing agents that are usable in this context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEENS and spans, e.g., TWEEN 80. Other solubilizers that are usable for the compositions of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

Suitable penetration enhancers include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propyleneglycol (PG), propyleneglycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, and the like. The permeation enhancer may also be a vegetable oil. Such oils include, for example, safflower oil, cottonseed oil and corn oil.

Suitable anti-irritants include, for example, steroidal and non-steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

Exemplary additional active agents according to some embodiments of present invention include, without limitation, one or more, or any combination of an anti-acne agent, an anti-aging agent, a wrinkle-reducing agent, a skin whitening agent, a sebum reducing agent, an anesthetic agent, an antipruriginous agent, an antineoplastic agent, an immunomodulator, an interferon, an antidepressant, an anti-histamine, a vitamin (e.g. A, C, E, B3, B5, K and their derivatives, in particular their esters), hyaluronic acid, a mineral (e.g., a dead sea mineral), a hormone and an anti-dandruff agent.

Exemplary additional active agents according to some embodiments of present invention include, without limitation, one or more, or any combination of a vitamin, a mineral (e.g., a dead sea mineral), a ceramide, hyaluronic acid, a protein (e.g., collagen, elastin), a hydroxy acid (e.g., alpha or beta hydroxyacid), a peptide, an amino acid, a sunscreen agent, a herb or plant extract, a fungus, a herb (e.g., a medicinal herb, a seaweed, an alga), urea and its hydroxylated derivatives, such as the N-(2-hydroxyethyl)urea, salicylic acid, sequestering agents (e.g. EDTA) and more.

According to specific embodiments, the cosmetic composition comprises a compound selected from the group consisting of glycerin, cetearyl alcohol, cetearyl glucoside, caprylic, capric triglyceride, isononyl isononanoate, Simmondsia Chinensis (jojoba) seed oil, Butyrospermum Parkii (Shea) Butter, Cocos Nucifera (coconut) oil, sodium polyacrylate, phenoxyethanol, caprylic glycol, tocopheryl acetate and perfume.

According to specific embodiments, the total amount of the bilayer-forming lipid and the polymeric compound is in a range of from 0.1 to 10, or from 0.1 to 5, or from 1 to 10, or from 1 to 5, % by weight of the total weight of the composition, including any intermediate values and subranges therebetween.

According to specific embodiments, the composition is in a form of a gel and the total amount of the bilayer-forming lipid and the polymeric compound is in a range of from 1 to 10, or from 1 to 5, % by weight of the total weight of the composition, including any intermediate values and subranges therebetween.

According to specific embodiments, the composition is in a form of a cream or an emulsion and the total amount of the bilayer-forming lipid and the polymeric compound is in a range of from 0.1 to 5, or from 0.1 to 1, % by weight of the total weight of the composition, including any intermediate values and subranges therebetween.

The formulations as described herein can be or be used to make up a cosmetic product and be used in cosmetic care.

Exemplary non-limiting products include, but are not limited to, an anti-acne product, an anti-diaper rush product, a shampoo, a body lotion, a body cream, a face cream, a face lotion, a face mask, a body and/or face wash product, a cleansing product (for hair and/or skin and/or mucosal tissue such as oral cavity or vagina), a hygienic product, an anti-pigmentation product, a make-up product, an anesthetic product, a sunscreen product.

According to specific embodiments, the cosmetic composition is used for skin care.

“Skin-care” means regulating and/or improving a skin condition. Some non-limiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin-care active” means a compound or combination of compounds that, when applied to skin, provide an acute and/or chronic benefit to skin or a type of cell commonly found therein.

Skin-care actives may regulate and/or improve skin or its associated cells (e.g., improve skin elasticity; improve skin hydration; improve skin condition; and improve cell metabolism).

“Skin-care formulation” means a formulation that includes a skin-care active and regulates and/or improves skin condition.

“Skin-care product” as used herein refers to a product that includes a skin-care composition or formulation. Some non-limiting examples of “skin-care products” include skin creams, moisturizers, lotions, and body washes. Other examples are provided hereinbelow According to an aspect of some embodiments of the present invention, there is provided a skin care product comprising the cosmetic or cosmeceutical composition or formulation, as described herein in any of the respective embodiments and any combination thereof.

Non-limiting exemplary skin care products include products for improving skin appearance and/or feel by providing a smoother, more even appearance and/or feel; products for increasing the thickness of one or more layers of the skin; products for improving the elasticity or resiliency of the skin; products for improving the firmness of the skin; products for reducing the oily, shiny, and/or dull appearance of skin, products for improving the hydration status or moisturization of the skin, products for improving the appearance of fine lines and/or wrinkles, products for improving skin exfoliation or desquamation, products for plumping the skin, products for improving skin barrier properties, products for improving skin tone, products for reducing the appearance of redness or skin blotches, and products for improving the brightness, radiancy, or translucency of skin. Such products are to be applied, for example, to a facial skin, the neck, the torso, and/or the ddcolletd.

The formulations and products as described herein can be used to treat, prevent (protect from) or reduce damage to a keratinous and/or mucosal tissue of a subject.

Medical, cosmetic or cosmeceutical conditions that can benefit from topical application of the compositions, formulations or products as described herein include, but are not limited to, damage to skin cells caused by UV radiation, skin aging, skin pigmentation, stress, extreme weather conditions, menopause, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, xeroderma pigmentosum, infections caused by pathogenic microorganisms, wounds, inflammation and/or pain, inflammatory diseases or disorders such as tinea pedis (athlete's foot), acne, tinea versicolor (a benign scaly skin condition), and thrombophlebitis, primary Raynaud's phenomenon (PRP), limited cutaneous systemic sclerosis (LCSSc), diabetic ulcer wounds, skin infections, eczema, rash, immunologically mediated systemic diseases, allergic response-mediated systemic diseases, viral blisters such as one caused by herpes, anal fissure, anal fissure pain, post Hirschprung surgery (for the treatment of obstructive symptoms), acute strangulated internal hemorrhoids, pain and symptoms of chronic extensor tendinosis of the elbow (tennis elbow).

According to some embodiments, the keratinous tissue is a facial tissue, a hair tissue, or a skin tissue (e.g., of the ddcolletd).

According to some embodiments, the mucosal tissue is a vaginal tissue.

According to some embodiments, the mucosal tissue is in the oral cavity.

According to some embodiments, the damage is associated with aging.

According to some embodiments, the damage is associated with a dermatological condition, for example, skin inflammation, and/or conditions such as atopic dermatitis, psoriasis, pigmentation, acne, eczema, xerosis, ichthyosis, keratosis, keratoderma, pruritus, dermatitis, neuro-dermatitis, dermatitis herpetiformis, actinic keratosis, hyper and inflamed keratosis, atopic eczema, melanoma, rosacea, urticaria, seborrheic dermatitis, skin cancer, and xeroderma pigmentosum.

According to some embodiments, any of the compositions, formulations, articles-of-manufacturing and/or products as described herein, is packaged in packaging material and identified in print, in or on the packaging material, for its intended use.

The compositions as described herein can be identified for use in preparing any of the formulations or products or articles-of-manufacturing as described herein.

The compositions as described herein can be identified for use in preparing any of the products or articles-of-manufacturing as described herein.

The products and/or articles-of-manufacturing as described herein can be identified for an intended use, as described herein in any of the respective embodiments, depending on the formulation that makes up the product or article, and on other ingredients that may be included in the product.

According to an additional or an alternative aspect of the present invention there is provided a method of performing a cosmetic care in a subject in need thereof, the method comprising applying to the skin of the subject an effective amount of the cosmetic composition disclosed herein, thereby performing the cosmetic care.

According to specific embodiments, the subject is a female subject.

According to specific embodiments, the subject is a baby (e.g. under 4 years old, under 3 years old).

According to specific embodiments, the subject is at least 20 years old, at least 30 years old, at least 40 years old, or at least 50 years old.

According to specific embodiments, the subject is an elderly subject, According to specific embodiments, the subject is at least 70 years old, or at least 80 years old.

As used herein, the term “subject” includes mammals, preferably human beings at any gender and of any age.

According to specific embodiments, the subject suffers from dry skin.

### Additional Definitions:

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, oxo, cyano, nitro, azo, azide, sulfonamide, carboxy, thiocarbamate, urea, thiourea, carbamate, amide, and hydrazine. The hydrocarbon can be an end group or a linking group, as these terms are defined herein. The hydrocarbon moiety is optionally interrupted by one or more heteroatoms, including, without limitation, one or more oxygen, nitrogen and/or sulfur atoms. In some embodiments of any of the embodiments described herein relating to a hydrocarbon, the hydrocarbon is not interrupted by any heteroatoms.

Preferably, the hydrocarbon moiety has 1 to 20 carbon atoms. Whenever a numerical range; e.g., “1 to 20”, is stated herein, it implies that the group, in this case the alkyl group, may contain 1 carbon atom, 2 carbon atoms, 3 carbon atoms, etc., up to and including 20 carbon atoms.

Herein, the term “alkyl” refers to any saturated aliphatic hydrocarbon including straight chain and branched chain groups. Preferably, the alkyl group has 1 to 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond. The cycloalkyl group can be an end group, as this phrase is defined herein, wherein it is attached to a single adjacent atom, or a linking group, as this phrase is defined herein, connecting two or more moieties.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Representative examples are piperidine, piperazine, tetrahydrofuran, tetrahydropyran, morpholine and the like. The heteroalicyclic group can be an end group, as this phrase is defined herein, wherein it is attached to a single adjacent atom, or a linking group, as this phrase is defined herein, connecting two or more moieties.

Herein, the terms “amine” and “amino” each refer to either a —NR′R″ group or a —N+R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group refers to a —N═N+=N-end group.

An “alkoxy” group refers to any of an —O-alkyl, —O-alkenyl, —O-alkynyl, —O-cycloalkyl, and —O-heteroalicyclic end group, as defined herein, or to any of an —O-alkylene, —O-cycloalkyl- and —O-heteroalicyclic-linking group, as defined herein.

An “aryloxy” group refers to both an —O-aryl and an —O-heteroaryl group, as defined herein, or to an —O-arylene.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic end group, as defined herein, or to any of an —S-alkylene-, —S-cycloalkyl- and —S-heteroalicyclic-linking group, as defined herein.

A “thioaryloxy” group refers to both an —S-aryl and an —S-heteroaryl group, as defined herein, or to an —S-arylene.

A “carbonyl” or “acyl” group refers to a —C(═O)—R′ end group, where R′ is defined as hereinabove, or to a —C(═O)— linking group.

A “thiocarbonyl” group refers to a —C(═S)—R′ end group, where R′ is as defined herein, or to a —C(═S)— linking group.

A “carboxy”, “carboxyl”, “carboxylic” or “carboxylate” group refers to both “C-carboxy” and “O-carboxy” end groups, as defined herein, as well as to a carboxy linking group, as defined herein.

A “C-carboxy” group refers to a —C(═O)—O—R′ group, where R′ is as defined herein.

An “O-carboxy” group refers to an R′C(═O)—O—group, where R′ is as defined herein.

A “carboxy linking group” refers to a —C(═O)—O—linking group.

An “oxo” group refers to a ═O end group.

An “imine” group refers to a ═N—R′ end group, where R′ is as defined herein, or to an ═N-linking group.

An “oxime” group refers to a ═N—OH end group.

A “hydrazone” group refers to a ═N—NR′R″ end group, where each of R′ and R″ is as defined herein, or to a ═N—NR′— linking group where R′ is as defined herein.

A “halo” group refers to fluorine, chlorine, bromine or iodine.

A “sulfinyl” group refers to an —S(═O)—R′ end group, where R′ is as defined herein, or to an —S(═O)— linking group.

A “sulfonyl” group refers to an —S(═O)₂—R′ end group, where R′ is as defined herein, or to an —S(═O)₂— linking group.

A “sulfonate” group refers to an —S(═O)₂—O—R′ end group, where R′ is as defined herein, or to an —S(═O)₂—O— linking group.

A “sulfate” group refers to an —O—S(═O)₂—O—R′ end group, where R′ is as defined as herein, or to an —O—S(═O)₂—O— linking group.

A “sulfonamide” or “sulfonamido” group encompasses both S-sulfonamido and N-sulfonamido end groups, as defined herein, as well as a sulfonamide linking group, as defined herein.

An “S-sulfonamido” group refers to a —S(═O)₂—NR′R″ end group, with each of R′ and R″ as defined herein.

An “N-sulfonamido” group refers to an R'S(═O)₂—NR″— end group, where each of R′ and R″ is as defined herein.

A “sulfonamide linking group” refers to a —S(═O)₂—NR′— linking group, where R′ is as defined herein.

A “carbamyl” group encompasses both O-carbamyl and N-carbamyl end groups, as defined herein, as well as a carbamyl linking group, as defined herein.

An “O-carbamyl” group refers to an —OC(═O)—NR′R″ end group, where each of R′ and R″ is as defined herein.

An “N-carbamyl” group refers to an R′OC(═O)—NR″— end group, where each of R′ and R″ is as defined herein.

A “carbamyl linking group” refers to a —OC(═O)—NR′— linking group, where R′ is as defined herein.

A “thiocarbamyl” group encompasses O-thiocarbamyl, S-thiocarbamyl and N-thiocarbamyl end groups, as defined herein, as well as a thiocarbamyl linking group, as defined herein.

An “O-thiocarbamyl” group refers to an —OC(═S)—NR′R″ end group, where each of R′ and R″ is as defined herein.

An “N-thiocarbamyl” group refers to an R′OC(═S)NR″— end group, where each of R′ and R″ is as defined herein.

An “S-thiocarbamyl” group refers to an —SC(═O)—NR′R″ end group, where each of R′ and R″ is as defined herein.

A “thiocarbamyl linking group” refers to a —OC(═S)—NR′— or —SC(═O)—NR′— linking group, where R′ is as defined herein.

An “amide” or “amido” group encompasses C-amido and N-amido end groups, as defined herein, as well as an amide linking group, as defined herein.

A “C-amido” group refers to a —C(═O)—NR′R″ end group, where each of R′ and R″ is as defined herein.

An “N-amido” group refers to an R′C(═O)—NR″— end group, where each of R′ and R″ is as defined herein.

An “amide linking group” refers to a —C(═O)—NR′— linking group, where R′ is as defined herein.

A “urea group” refers to an —N(R′)—C(═O)—NR″R′″ end group, where each of R′, R″ and R″ is as defined herein, or an —N(R′)—C(═O)—NR″— linking group, where each of R′ and R″ is as defined herein.

A “thiourea group” refers to an —N(R′)—C(═S)—NR″R′″ end group, where each of R′, R″ and R″ is as defined herein, or an —N(R′)—C(═S)—NR″— linking group, where each of R′ and R″ is as defined herein.

A “nitro” group refers to an —NO₂ group.

A “cyano” group refers to a —C—N group.

The term “phosphonyl” or “phosphonate” describes a —P(═O)(OR′)(OR″) group, with R′ and R″ as defined herein, or a —P(═O)(OR′)—O—linking group, with R′ as defined herein.

The term “phosphate” describes an —O—P(═O)(OR′)(OR″) end group, with each of R′ and R″ as defined herein, or an —O—P(═O)(OR′)—O—linking group, with R′ as defined herein.

The term “phosphinyl” describes a —PR′R″ end group, with each of R′ and R″ as defined herein, or a —PR′—linking group, with R′ as defined herein.

The term “hydrazine” describes a —NR′—NR″R′″ end group, where R′, R″, and R′″ are as defined herein, or to a —NR′—NR″— linking group, where R′ and R″ are as defined herein.

As used herein, the term “hydrazide” describes a —C(═O)—NR′—NR″R′″ end group, where R′, R″ and R′″ are as defined herein, or to a —C(═O)—NR′—NR″— linking group, where R′ and R″ are as defined herein.

As used herein, the term “thiohydrazide” describes a —C(═S)—NR′—NR″R′″ end group, where R′, R″ and R′″ are as defined herein, or to a —C(═S)—NR′—NR″— linking group, where R′ and R″ are as defined herein.

A “guanidinyl” group refers to an —RaNC(═NRd)-NRbRc end group, where each of Ra, Rb, Re and Rd can be as defined herein for R′ and R″, or to an —R′NC(═NR″)—NR′—linking group, where R′, R″ and R′″ are as defined herein.

A “guanyl” or “guanine” group refers to an R′″R″NC(═NR′)—end group, where R′, R″ and R′″ are as defined herein, or to a —R″NC(═NR′)—linking group, where R′ and R″ are as defined herein.

For any of the embodiments described herein, the compound described herein may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the intended activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

As used herein the term “about” refers to ±10%, and optionally ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

Distearoylphosphatidylethanolamine (DSPE) and Dipalmitoylphosphatidylethanolamine (DPPE), exemplary bilayer-forming lipids, were obtained from Lipoid.

Hydrogenated soy phosphatidylcholine (HSPC), an exemplary bilayer-forming lipid, was obtained from Lipoid GmbH.

PC S 100 (Phosphatidylcholine from soybean), an exemplary bilayer-forming lipid, was obtained from Lipoid GmbH.

MLV's are multilamellar vesicles (liposomes) comprising a phospholipid or a phospholipid and LPC as described herein in any of the respective embodiments.

Carbomer—Carbopol based gel—was obtained from Lubrizol advanced materials Europe BV, Cat No. CBP1058.

HA—hyaluronic acid—was obtained from ‘Contipro a.s’: Catalogue number: 814-10-01 & 814-12-01.

Particles size was determined by dynamic light scattering (DLS) measurements using a Zetasizer Nano instrument by Malvern Panalytical. Liposome samples were diluted ×10 in PBS to a final volume of 1 mL, transferred to a plastic cuvette and equilibrated to 25 °C, followed by 3 replicate measurements. The mean Z-average diameter and mean polydispersity index (PDI) were reported along with their standard deviations.

Z-average was determined using a Zetasizer Nano instrument by Malvern Panalytical. Samples were diluted ×100-200 in water to a final volume of 800 μL, transferred to a disposable folded capillary cuvette and equilibrated to 25 °C, followed by 3 replicate measurements. The Z-average (mean) and its standard deviation are reported.

HPLC measurements were performed using Agilent 1100 system equipped with an evaporative light scattering (ELS) detector using a reversed phase C8 column (Kinetex C8, 150×4.6 mm, 5 μm, 100A) equilibrated to 30 °C.

GPC measurements were performed using an Agilent 1100 system equipped with a refractive index detector using a combination of PFG columns (PFG Column Guard+100 Å PFG single pore+300 Å PFG single pore) equilibrated to 40 °C.
Other methods are described hereinunder.

### Example 1

### Lipid Polymer Conjugates (LPCs) and Liposome Formulations Comprising Same

### Lipid Polymer Conjugates (LPCs):

Lipid polymer conjugates (LPCs) are prepared as generally described in U.S. Provisional Patent Application No. 63/402,097, and/or in a co-filed PCT International Patent Application having Attorney's Docket No. 97558, which claims the benefit of priority of U.S. Provisional Patent Application No. 63/402,097, by the present assignee, which are incorporated by reference as if fully set forth herein.

According to some of any of the embodiments described herein, a process of preparing an LPC according to the present embodiments comprises contacting an initiator compound having Formula V:

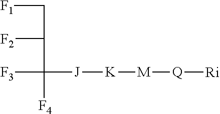

wherein:
F₁, F₂, F₃, F₄, J, K, M and Q are as defined for Formula IV; and
Ri is an electron transfer functional group,
with a plurality of monomers that form the —[Y-L-Z]n-[Y]m- polymeric backbone, wherein Y, L, Z, n and m are as described herein for Formula I,
under conditions that promote atom transfer radical polymerization (ATRP), preferably under conditions that promote ARGET-ATRP.

According to some of any of the embodiments described herein, Ri can be any functional group that is suitable for electron transfer radical polymerization, and is typically a group that is capable of forming a stable radical on its own. Exemplary such groups include halogens (halo), preferably chloro or bromo, more preferably bromo, although any other suitable groups are contemplated.

The term “stable radical” as used herein encompasses any chemical species that comprises an unpaired electron within its molecular or atomic structure but is relatively long-lived and less reactive compared to typical radicals. Typically, a stable radical is such that is capable of stabilizing the unpaired electron energetically.

Conditions that promote ATRP include any conditions known in the art, typically in the presence of radical forming reagents such as CuX′ or CuX′2, wherein X′ is typically halogen, and a suitable ligand.

According to some of any of the embodiments described herein, the process is effected under conditions that promote ARGET-ATRP. Exemplary such conditions include any conditions known in the art, typically in the presence of CuX′₂, wherein X′ is typically halogen, a suitable ligand, and a reducing agent. Examples of safe, cheap and commercially available reducing agents that can be employed in ARGET-ATRP include, without limitation, ascorbic acid (vitamin C), sodium ascorbate (NaAsc), Calcium Ascorbate (Ca2Asc), and hydrazine (mono hydrate or dissolved in ethanol), tin-2-ethyhexanoate (Tin-2EH) and glucose. For polymerization of MPC, both CuBr₂ and CuCl₂ (preferably anhydrous) work well together with TPMA as an exemplary ligand, while the constant reduction of the catalyst is performed using ascorbic acid [see, for example, Adler et al., Biomaterials Science, 2021, 9, 5854-5867]. Other ligands are also contemplated, including, for example, N,N,N′,N′-tetrakis(2-pyridinylmethyl)-1,2-ethanediamine (TPEN; CAS No. 16858-02-9).

Suitable solvents for carrying out ATRP or ARGET-ATRP processes include, without limitations, polar solvents such as alcohols (e.g., methanol and/or ethanol).

According to some of any of the embodiments described herein, the process is effected at a temperature in a range of from 10 °C to 50 °C, or from 15 °C to 50 °C, or from 15 °C to 30 °C, including any intermediate value and subranges therebetween. In some of any of the embodiments described herein, the process is effected at room temperature (i.e., ambient temperature; from about 20 °C to about 25 °C).

According to some of any of the embodiments described herein, contacting the initiator compound with the plurality of monomers is effected for a time period of from about 1 to about 48 hours, or from about 3 to about 48 hours, or from about 3 to about 36 hours, or from about 3 to about 24 hours, from about 4 to about 48 hours, or from about 4 to about 36 hours, or from about 4 to about 24 hours, from about 6 to about 48 hours, or from about 6 to about 36 hours, or from about 6 to about 24 hours including any intermediate value and subranges therebetween.

Exemplary procedures for performing ATRP process and ARGET-ATRP processes are described hereinunder. These procedures can be manipulated as desired by selecting an initiator compound, a mol ratio of the plurality of monomers to the initiator, by manipulating the catalyst solution, the ligand and/or the reducing, and by selecting a synthetic protocol as exemplified for Procedures 2 and 3.

According to some of any of the embodiments described herein, the process is effected by contacting a catalyst solution that comprises a catalyst and a ligand with a solution comprising the initiator compound, preferably under inert atmosphere (e.g., argon), and with a solution of the plurality of monomers, at a mol ratio to the initiator compound selected to provide a desired length (number of repeating backbone units) of the obtained LPC.

According to some of any of the embodiments described herein, a mol ratio of the catalyst and the initiator is about 1:1.

According to some of any of the embodiments described herein, the process is effected by ARGET-ATRP, using procedures known in the art. In some of these embodiments, a catalyst solution is prepared by dissolving the catalyst and the ligand in a polar solvent (e.g., an alcoholic solvent such as MeOH or EtOH, preferably EtOH). In an exemplary procedure (e.g., Procedure 2), the catalyst solution is added to a mixture of the initiator compound, a reducing agent and a plurality of monomers, in apolar solvent as described herein (e.g., an alcoholic solvent). In another exemplary procedure (e.g., Procedure 3), the catalyst solution is added to a mixture of the initiator compound, and a plurality of monomers, in a polar solvent as described herein (e.g., an alcoholic solvent), and a reducing agent is thereafter added.

In some of any of the embodiments described herein, the mol ratio of the monomer (plurality of monomers) as described in any of the respective embodiments and in any combination thereof, and the initiator compound as described herein, is in a range of from 5:1 to 200:1, or from 10:1 to 150:1, or from 20:1 to 100:1, or from 30:1 to 75:1, including any intermediate values and subranges therebetween. This mol ratio determines the number of repeating units in the obtained polymeric compound (LPC).

In some embodiments, a mol ratio of 50:1 or lower (e.g., about 30:1 or about 25:1) provides short LPCs as described herein in of the respective embodiments, comprising less than 100, or less than 80, repeating units in the polymeric moiety (indicated by variable n in Formula I as described herein).

In some embodiments, a mol ratio of 60:1 or higher (e.g., from about 60:1 to about 80:1) provides longer LPCs as described herein in of the respective embodiments, comprising at least 80 repeating units (e.g., 80-120) in the polymeric moiety (indicated by variable n in Formula I as described herein).

According to some of any of the embodiments described herein, the process further comprises isolating the polymeric compound.

Isolating the obtained polymeric compound (e.g., a polymeric compound having Formula I as described herein in any of the respective embodiments and any combination thereof), can be effected by any work up process known in the art, preferably in the context of ATRP processes, including, for example, TFF, column chromatography and/or precipitation. Exemplary such procedures are described hereinunder.

According to some of any of the embodiments described herein, isolating the polymeric compound does not involve acidification, namely, exposing the polymeric compound to an acidic environment.

According to some of any of the embodiments described herein, isolating the polymeric compound is performed by precipitation, namely, precipitating the polymeric compound from the polymerization reaction mixture by means of, for example, contacting the mixture with an anti-solvent in which the polymeric compound is not soluble, at a ratio in a range of from 2:1 to 50:1, anti-solvent: reaction mixture, including any intermediate values and subranges therebetween.

Exemplary anti-solvents include, but are not limited to ketones such as acetone, dimethoxyethane (DME), chloroform (CHCl₃), dichloromethane (DCM), tetrachloroethylene (C₂Cl₄), dimethylcarbonate (DMC), diethylcarbonate (DEC), and methyl tert-butyl ether (MTBE).

In exemplary embodiments, the anti-solvent is a ketone, for example, acetone.

According to some of any of the embodiments described herein, isolating the polymeric compound further comprises, prior to and/or subsequent to the precipitation, column chromatography.

As an exemplary LPC, DPPE-Ph-pMPC was used, with n featuring varying values, as indicated hereinafter.

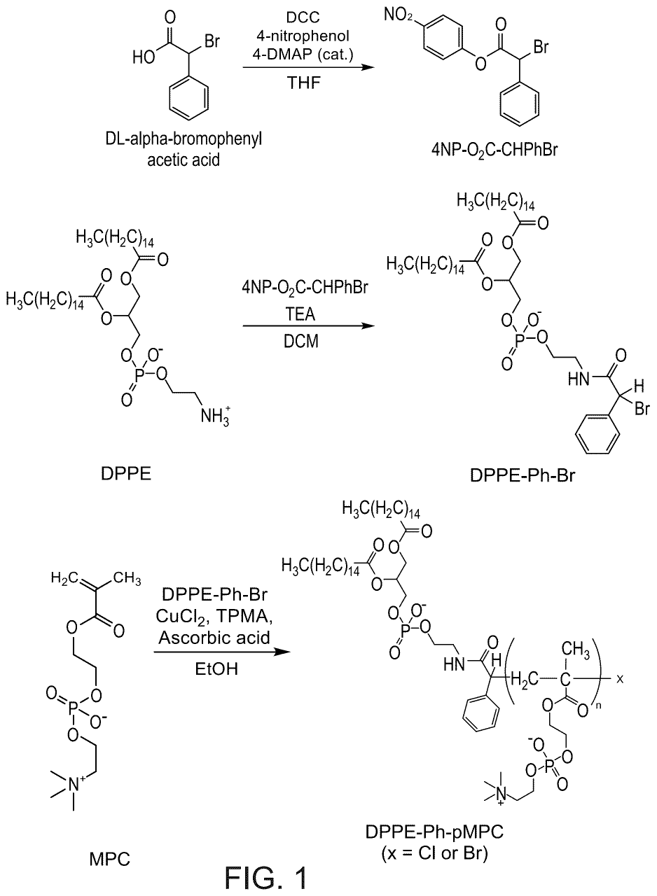

The chemical structure and an exemplary general synthetic protocol (Procedure 2) used for preparing this exemplary LPC are depicted in FIG. 1.

### Preparation of DPPE-pH-Br Initiator (Atom-Transfer Radical Polymerization (ATRP) Initiator):

In an exemplary procedure, DPPE-Ph-Br was prepared via a two-step reaction as follows: (a) Steglich esterification of DL-α-bromophenylacetic with 4-nitrophenol to afford the active 4-nitrophenol ester (4NP—O₂C—CHPhBr); and (b) reacting the resulting ester with DPPE to obtain DPPE-Ph-Br, as depicted in FIG. 1. Reacting the resulting ester with other glycerophospholipids can provide additional Ph-Br-based initiators.

(a) Synthesis of 4-nitrophenyl 2-bromo-2-phenylacetate (4NP—O₂C—CHPhBr):

DL-α-bromophenylacetic acid (4.30 grams, 20 mmol, 1.0 mol equivalent), 4-nitrophenol (3.06 grams, 22 mmol, 1.1 mol equivalent) and 4-DMAP (0.244 gram, 2 mmol, 0.1 mol equivalent) were weighed into a round bottomed flask equipped with a magnetic stirring bar. THF (20 mL per 1 gram of DL-α-bromophenylacetic acid) was added to the flask and stirring was turned-on to completely dissolve all solids. The flask was thereafter sealed, cooled to about 5 °C using an ice-bath, and dicyclohexylcarbodiimide (DCC; 4.54 grams, 22 mmol, 1.1 mol equivalent) was added. After 10 minutes of stirring, the ice-bath was removed, and the flask was allowed to reach room temperature. The reaction mixture was allowed to stir for 3 more hours at ambient temperature and then its content was gravitationally filtered-off into an evaporation flask using a common filtration paper. The reaction flask and the filtered urea byproduct were washed twice with DCM (25 mL) and the combined filtrates were evaporated to dryness using a rotary evaporator. The obtained yellowish oily crude mixture was re-dissolved in DCM (10-15 mL) and loaded on a silica column with DCM as eluent. Collected fractions that contained the product (identified using TLC with UV light at 254 nm, eluent=DCM, Rf=0.95) were combined, evaporated to dryness, and placed under high vacuum overnight. The product was obtained as a clear and slightly yellowish oil, and upon overnight drying under high vacuum it crystalized to give 5.05 grams of a pale yellow solid in 75% yield.

(b) Synthesis of 2,3-bis(palmitoyloxy)propyl (2-(2-bromo-2-phenylacetamido)ethyl) phosphate (DPPE-Ph-BrATRP Initiator):

4NP—O₂C—CHPhBr (5.05 grams, 15.0 mmol, 1.2 mol equivalent) was dissolved using DCM (15 mL per 1 gram 4NP—O₂C—CHPhBr) in a round bottomed flask equipped with a magnetic stirring bar. Then, DPPE (8.66 grams, 12.5 mmol, 1.0 mol equivalent) was added to the flask and washed down with additional DCM (10 mL per 1 gram DPPE). TEA (4.2 mL, 30.0 mmol, 2.4 mol equivalents) was added and the flask was heated to 50 °C using a water bath to afford gentle solvent reflux. At the beginning of the reaction, a foam was obtained in the solution, which was cleared following one hour of reflux and vigorous stirring. The reaction was refluxed for additional 2 hours and was then allowed to cool to room temperature. The content of the flask was transferred into a separation funnel and the organic phase was washed thrice with equivalent volumes of 1 Molar (M) HCl for each wash. The combined aqueous acidic phase was extracted twice with DCM (50 mL). The combined organic DCM phase was dried over anhydrous Na2SO₄, filtered, and evaporated to 30-50 mL. The DPPE-Ph-Br product was precipitated by adding the concentrated DCM solution to a laboratory bottle filled with stirring MeOH (75 mL per 1 gram of DPPE) and placing this bottle in the fridge for at least 2 hours. The precipitated product was filtered-off, washed twice with cold MeOH (50 mL) and allowed to dry with suction still turned-on for at least 10 minutes. Finally, the precipitate was dried overnight under high vacuum to give 9.82 grams of DPPE-Ph-Br in 88% yield.

In another exemplary procedure, DPPE-Ph-Br was prepared as follows:

(a) Activation of αBPA with NHS Towards Coupling Reaction with DPPE:

DL-α-bromophenylacetic acid (αBPA, 7.53 grams, 35.0 mmol, 1.4 mol equivalent to DPPE) and N-hydroxysuccinimide (NHS, 4.32 grams, 37.5 mmol, 1.5 mol equivalent to DPPE) were added into a 100 mL round-bottomed flask equipped with a magnetic stirrer and a powder funnel. Tetrahydrofuran (THF, 40 mL) was added through the powder funnel and stirring was turned on to completely dissolve both solids. Diisoproylcarbodiimide (DIC, 5.1 mL, 32.5 mmol, 1.3 mol equivalent to DPPE) was added thereafter to the flask using a syringe, the flask was sealed, and the reaction was allowed to stir for 1 hour at room temperature. Then, the precipitated DIC-urea byproduct was filtered-off gravitationally through a standard filtration paper and the reaction flask and filtration paper were washed with 5 mL THF. The obtained clear solution was kept aside for the next step.

(b) Reacting DPPE with the NHS Ester of αBPA:

In parallel, a 500 mL round-bottomed flask was equipped with a magnetic stirrer and a powder funnel. 1,2-dipalmitoyl-sn-glycero-3-phosphoethanolamine (DPPE, 17.3 grams, 25 mmol) was added to the flask, following by addition of chloroform (CHCl₃, 175 mL) through the powder funnel. Then, triethylamine (TEA, 3.8 mL, 27.5 mmol, 1.1 mol equivalent to DPPE) was added to the flask using a syringe, and the contents of the flask were strongly stirred while heating the flask to 70 °C. After complete dissolution was achieved, the flask was allowed to gradually cool down to 35 °C, and then the clear NHS ester solution from Step (a) was added in a single portion, and the reaction was allowed to proceed for 1 hour at 35 °C. Thereafter, the flask was allowed to cool down to room temperature, e CHCl₃ was evaporated under reduced pressure and the obtained oily residue was transferred into a 1 L separation funnel using a total of 500 mL Ethyl Acetate (EtOAc). The organic phase in the separation funnel was washed thrice with HCl 1M (250 mL), thrice with Brine (250 mL), dried over anhydrous MgSO₄, and filtered gravitationally using a standard filtration paper into a 1 L lab bottle to separate any solid particulates. The lab bottle with the clear EtOAc solution was placed in a −20 °C freezer for 2 hours, during which the DPPE-Ph-Br product precipitated as a white solid. DPPE-Ph-Br was filtered-off using a Buchner filtration system and the collected product was washed twice with cold EtOAc (25 mL). The product was dried to constant weight under high vacuum. A yield of 80-90% of pure DPPE-Ph-Br was obtained.

As the above procedure includes in situ activation of αBPA without the need to perform any purification to the active ester, the DPPE-Ph-Br can alternatively be prepared in a one-pot reaction. Activation is performed by turning αBPA into an active NHS ester using diisoproylcarbodiimide (DIC) as a coupling reagent.

### Synthesis of DPPE-Ph-pMPC Via ARGET-ATRP:

MPC was polymerized from the exemplary DPPE-Ph-Br initiator via ARGET-ATRP using anhydrous CuCl₂ as the catalyst, tris(2-pyridylmethyl) (TPMA) as the ligand, and ascorbic acid or Sodium Ascorbate (NaAsc) as the reducing agent, to obtain DPPE-Ph-pMPC (see, for example, FIG. 1).

Generally, catalysts, reducing agents and ligands and solutions thereof can alternatively be replaced by others known in the art in the context of ARGET-ATRP. The initiator can also be replaced by any initiator that corresponds to the desired LPC, as described herein (see, for example, Formula V).

Generally, an amount of the ligand is about 2 mol equivalents relative to the CuCl₂ catalyst; an amount of the reducing agent is about 10 mol equivalents relative to the CuCl₂ catalyst; an amount of the DPPE-Ph-Br is about 1 mol equivalent relative to the CuCl₂ catalyst; and an amount of the MPC is pre-determined in accordance with a desired number of the repeating units (n). The length of the LPC is determined by the mol ratio of DPPE-Ph-Br to MPC, and accordingly, the amount of the MPC added to the reaction was selected. For example, a mol ratio of 1:25 DPPE-Ph-Br:MPC resulted in LPC comprising about 50-80 or 50-60 repeating units. A mol ratio of 1:50 or 1:60 resulted in LPC comprising about 80-120 or 110-120 repeating units.

In all of the synthetic protocols described herein for preparing a lipid-polymer conjugate, the type of the lipid moiety can be manipulated by preparing a respective initiator (which comprises the selected lipid moiety). Further, the length of the polymeric moiety (number of repeating units of e.g., MPC) is controlled by the mol equivalents of the selected monomer (e.g., MPC) with respect to the initiator (a mol ratio of the monomer relative to the initiator).

The following exemplary general procedure was used, employing the following catalyst solution: CuX₂ (X=Cl or Br, 0.10 mol equivalent to the initiator) was dissolved in an alcoholic solvent (e.g., MeOH or EtOH, preferably EtOH). TPMA (0.20 mol equivalent to the initiator) was added to the CuCl₂ green solution, and the obtained metal-ligand complex solution was mixed for at least 15 minutes.

In one exemplary procedure (also referred to herein as Procedure 2), into an oven-dried round-bottomed Schlenk flask equipped with a magnetic stirring bar, an initiator (1 mol equivalent) and Ascorbic Acid (1.41 mol equivalent to initiator) were added, followed by addition of a solution of MPC (25-100 mol equivalents to initiator; e.g., 30 mol equivalents to initiator) in the selected solvent (e.g., EtOH). The total solvent (e.g., EtOH) volume used in the reaction was set according to the amount of MPC to give a concentration of 15% w/v. The Catalyst solution was then added to the Schlenk flask, the flask was sealed with rubber septum and all contents were mixed together to form a greenish clear solution.

In an alternative procedure (also referred to herein as Procedure 3), into an oven-dried round-bottomed Schlenk flask equipped with a magnetic stirring bar, an initiator (1 mol equivalent) was added, followed by addition of a solution of MPC (25-100 mol equivalents to initiator; e.g., 30 mol equivalents to initiator) in the selected solvent (e.g., EtOH), as above, and an addition of the catalyst solution. The flask was sealed with rubber septum and all contents were mixed together to form a greenish clear solution, and, after all of the solids completely dissolved, Sodium Ascorbate (1.00 mol equivalent to the initiator) was added.

Once all components were introduced to the flask, it was purged from oxygen by bubbling Argon gas for 30 minutes through the solution. The Schlenk valve was thereafter tightly closed, and the polymerization mixture was allowed to stir at ambient temperature for 2-24 (e.g., 5-6) hours.

In both Procedures 2 and 3, polymerization was quenched by opening the flask to air. The obtained solution was optionally filtered-off using a Buchner funnel equipped with a Whatman paper. The obtained solution (or filtrate) was added dropwise into acetone while stirring, to precipitate the polymer product as a white solid. The volume of acetone was set to be 10 times greater than the volume of the alcoholic solvent (e.g., EtOH). The formed slurry was allowed to vigorously stir for additional 30 minutes, and the precipitate was thereafter filtered-off through a glass funnel, and the precipitation vessel and the polymer were washed thrice with acetone. The obtained polymer (LPC), which had a paste-like texture, was purified by dissolving it in EtOH and/or deionized water (15 mL per 1 gram of LPC), while optionally removing impurities by filtration. When EtOH was used, the solvents were evaporated and the residue was dissolved in purified water, filtered from insoluble impurities, and further purified by TFF (8-10 diafiltrations with deionized water). When water was used, the obtained aqueous solution was filtered from insoluble impurities and then further purified using TFF (8-10 diafiltrations with DIW). The aqueous LPC solutions were placed in a −80 °C freezer for at least 2 hours and then lyophilized for at least 48 hours (e.g., 96 hours).

The number of units in the obtained LPC was determined based on the Mw of the product, which was determined by GPC analysis.

All the tested LPCs were prepared using DPPE-Ph-Br as an initiator compound, following Procedure 2 as described herein.
IM22-1053 was prepared according to the above procedure, using 30 mol equivalents MPC relative to the DPPE-Ph-Br.
IM22-1054 was prepared according to the above procedure, using 80 mol equivalents MPC relative to the DPPE-Ph-Br.
IM22-1055 was prepared according to the above procedure, using 30 mol equivalents MPC relative to the DPPE-Ph-Br.
IM22-1056 was prepared according to the above procedure, using 30 mol equivalents MPC relative to the DPPE-Ph-Br.
IM22-1063 was prepared according to the above procedure, using 25 mol equivalents MPC relative to the DPPE-Ph-Br.

Table 1 below presents the characterizing properties of the obtained LPCs.

TABLE 1

| | | MPC | |
|---|---|---|---|
| Batch | M.W. | Units | PDI |
| IM22-1053 | 18400 | 53 | 1.1 |
| IM22-1063 | 20563 | 61 | 1.1 |
| IM22-1054 | 34400 | 104 | 1.1 |
| IM22-1056 | 25124 | 75 | 1.1 |
| IM22-1055 | 27200 | 89 | 1.1 |

Liposomes formed of DPPE-Ph-pMPC of different lengths and dipalmitoyl phosphatidylcholine lipids (DMPC), hydrogenated soy phosphatidylcholine (HSPC), Phosphatidylcholine from soybean (PC S 100), as a phospholipid were prepared and added to various cosmetic formulations as described in Example 2 below.

Liposome stock solutions were generally prepared as follows. Protocols for each formulation are described in further details in Example 2 that follows.

Ethanol dissolving of the lipids—55 grams Ethanol AR was added to a 250 ml flask and heated to a temperature in a range of 30-70 °C. The selected LPC was weighed and added to the flask followed by the selected phospholipid. Reference liposome formulations were prepared without LPC. A 2.5 cm egg-shaped magnet was added to the flask and ingredients were mixed at a temperature in a range of 30-70 °C until a homogenous clear solution was obtained (typically in about 20 minutes).

Ethanol evaporation—The solution was evaporated using a ‘BUCHI’ Roto-vapor (water bath set to 35 °C, −2 inclination, 250 RPM) until dry, followed by a second validating evaporation for 15 minutes, forming a dry lipid cake.

Hydration in water—55 grams of di-ionized water were added to the dried flask along with a 2.5 cm magnet and the flask was stirred for 20-70 minutes at a hydration temperature in a range of 30-70 °C.

Homogenization—The solution was placed inside a 250 ml lab bottle immersed in a 35 °C bath, to be homogenized at 7-9K RPM for 30-60 minutes.

Table 2 below describes the preparation parameters and characterizing properties of exemplary liposomal formulations.

TABLE 2

| | | | | | | | Homo- | | |
|---|---|---|---|---|---|---|---|---|---|
| | | | | Prcoess | Hydration | Hydration | genization | Z- | |
| | | | | Temp | Time | Temp | Time | average | |
| # | Formulation | LPC | Lipid | (°C) | (Minutes) | (°C) | (minutes) | [nm] | PDI |
| 1 | IM22-782 | IM22-1063 | DMPC | 35 | 60 | 35 | — | — | — |
| 2 | IM22-780 | IM22-1056 | PC S | 65 | 60 | 65 | — | 1609 ± | 0.76 ± |
| | | | 100 | | | | | 400 | 0.40 |
| 3 | IM22-778 | IM22-1055 | HSPC | 65 | 60 | 65 | 40 | 900 ± | 1.00 |
| | | | | | | | | 31 | |
| 4 | IM22-777 | IM22-1054 | HSPC | 65 | 60 | 65 | 30 | 420 ± | 0.304 ± |
| | | | | | | | | 2.6 | 0.02 |
| 5 | IM22-754 | IM22-1054 | DMPC | 33 | 120 | 34 | — | 677 ± | 0.622 ± |
| | | | | | | | | 41 | 0.03 |
| 6 | IM22-744 | IM22-1054 | DMPC | 26 | 120 | 27 | — | 780 ± | 0.477 ± |
| | | | | | | | | 6.7 | 0.15 |
| 7 | IM22-743 | — | HSPC | 60 | 60 | 60 | 40 | 3401 ± | 0.702 ± |
| | | | | | | | | 523 | 0.26 |
| 8 | IM22-736 | — | DMPC | 38 | 60 | 38 | 60 | 1461 ± | 0.321 ± |
| | | | | | | | | 150 | 0.27 |
| 9 | IM22-732 | IM22-1053 | DMPC | 37 | 60 | 37 | 60 | 564 ± | 0.627 ± |
| | | | | | | | | 20 | 0.04 |

### Example 2

### Cosmetic Gel Formulations

The common ingredient of all formulations was a Carbomer gel. For the gel preparation, a pre-made gel stock (1.5% Carbopol® content) was generally prepared according to the following: 15 grams Carbopol® 990 was added to di-ionized water (930 grams) at room temperature and homogenized at 9K RPM for 7 minutes, followed by the addition of preservatives (40 grams ‘ISCAGARD OP’) and mixing for 1 minute. Following, 15 grams of tri-ethanolamine was added to neutralize the acidic Carbomer.

For a gel comprising common moisturizers such as glycerine, 37.5 grams of the stock gel was mixed with 9.4 grams glycerin and 140.8 grams di-ionized water using an over-head stirrer equipped with a stainless steel 6 cm wide ‘anchor’ type rod.

Samples comprising multilamellar vesicles composed of a phospholipid only (e.g., dipalmitoyl phosphatidylcholine; DMPC) or of a phospholipid and LPC as described herein were prepared by missing an amount of the stock gel and an amount of an homogenized solution of the lipids (prepared as described in Example 2), using an overhead stirrer equipped with a stainless steel 6 cm wide ‘anchor’ type rod.

The following presents preparation protocols of exemplary gel formulations:

### A Reference Gel Comprising HSPC Only (Also Referred to Herein as Formulation “IM22-743”; Table 2, Entry 7):

A gel formulation comprising Hydrogenated soy phosphatidylcholine lipids (HSPC), mixed with Carbomer gel (Carbopol®, di-ionized water, Tri-ethanolamine, and preservatives), resulting in 50.64 mM gel at a 4% w/w final gel lipid concentration was prepared as follows:

Gel preparation—a pre-made gel stock (IM22-728-1.5% Carbopol® content) was prepared as follows: 6 grams Carbopol® 990 was added to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine was added to neutralize the acidic Carbomer to a pH of 6.17.

Ethanol dissolving of the lipids—55 grams Ethanol AR at 57 °C was added to a 250 ml flask. HSPC (2.75 grams) was added along with a 2.5 cm egg-shaped magnet. Mixing was performed at 56 °C for 30 minutes at the evaporator until complete dissolution was obtained.

Ethanol evaporation—The solution was evaporated using a ‘BUCHI’ Roto-vapor (water bath set to 56 °C, 250 RPM) until dry, followed by a second verifying evaporation, forming a dry lipid cake.

Hydration in water—55 grams of di-ionized water at 60 °C were added to the dried flask which was mixed via an evaporator for 1 hour at 60 °C.

Homogenization—The solution was placed inside a cylinder immersed in a 65 °C bath and homogenized at 9K RPM for 40 minutes using a narrow ‘KINEMATICA’ knife, at that temperature.

Gel embedding was performed 12 hours following the homogenization. 12.5 grams of the stock gel was mixed with about 50 grams of the homogenized solution using an over-head stirrer equipped with a stainless steel ‘anchor’ type rod.

The resulting MLVs DLS analysis showed a Z-average of 3401±532 nm, PDI of 0.702±0.26. See, Table 2, Entry 7 in Example 1 hereinabove.

### a Reference Gel Comprising DMPC Only (Also Referred to Herein as Formulation “IM22-736”; Table 2, Entry 8):

A gel formulation comprising dipalmitoyl phosphatidylcholine lipid (DMPC), mixed with Carbomer gel (Carbopol®, di-ionized water, Tri-ethanolamine, and preservatives), resulting in 59.04 mM gel at a 4% w/w final gel lipid concentration was prepared as follows:
Gel preparation—a pre-made gel stock (IM22-728-1.5% Carbopol® content) was prepared as follows: 6 grams Carbopol® 990 was added to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine was added to neutralize the acidic Carbomer to a pH of 6.17.
Ethanol dissolving of the lipids—55 grams Ethanol AR at 37 °C was added to a 250 ml flask. DMPC (2.75 grams) was added to the flask, and mixing was performed at 37 °C at the Roto-evaporator until complete dissolution was obtained.
Ethanol evaporation—The solution was evaporated using a ‘BUCHI’ Roto-vapor (water bath set to 38 °C, 250 RPM) until dry, followed by a second verifying evaporation, forming a dry lipid cake.
Hydration in water—55 grams of di-ionized water at 38 °C were added to the dried flask and mixing was effected for 1 hour at the above temperature.
Homogenization—The solution was placed inside a cylinder immersed in a 38 °C bath, and homogenized for 2 hours.

The resulting MLVs DLS analysis showed a Z-average of 1461±150 nm, PDI of 0.321±0.27. See, Table 2, Entry 8 in Example 1 hereinabove.

Gel embedding was performed using 12.5 grams of the stock gel mixed with 50.20 grams of the homogenized solution using an over-head stirrer equipped with a stainless steel ‘anchor’ type rod.

### A Reference Gel Comprising 5% w/w Glycerine (Also Referred to Herein as Formulation “IM22-587”):

A reference 5% w/w glycerin gel formulation comprising Carbomer gel (Carbopol®, di-ionized water, Tri-ethanolamine, and preservatives), Glycerine & Di-ionized water. The resulting gel serves as a moisturizing reference herein while incorporation 5% w/w glycerine in any cosmetic preparation serves as common moisturizing standard.

Gel preparation—a pre-made gel stock (IM22-579-1.5% Carbopol® content) was prepared as follows: 15 grams Carbopol® 990 was added to di-ionized water (930 grams) at room temperature and the mixture was homogenized at 9K RPM for 7 minutes, followed by the addition of preservatives (40 grams Phenoxyethanol & Caprylyl Glycol mixture) and mixing. Thereafter, 15.00 grams of tri-ethanolamine was added to neutralize the acidic Carbomer to a pH of 5.72.

The final preparation was performed by adding 37.49 stock gel (IM22-579) with 9.39 grams Glycerin USP with 140.8 grams Di-ionized water and mixing using over-head stirrer equipped with a stainless steel ‘anchor’ type rod, resulting in a pH of 6.39.

### A Gel Comprising Liposomes Based on LPC (Long) and HSPC (Also Referred to Herein as Formulation IM22-777; Table 2, Entry 4):

A gel formulation comprising multilamellar vesicles composed of Hydrogenated soy phosphatidylcholine lipids (HSPC), the LPC IM22-1054 (see, Table 1) and water; mixed with Carbomer gel (Carbopol®, di-ionized water, Tri-ethanolamine, and preservatives), resulting in 44.80 mM gel at a 4% w/w final gel lipid concentration was prepared as follows:
Gel preparation—a pre-made gel stock (IM22-728-1.5% Carbopol® content) was prepared by adding 6 grams Carbopol® 990 to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine were added to neutralize the acidic Carbomer to a pH of 6.17.
Ethanol dissolving of the lipids—55 grams Ethanol AR at 65 °C were added to a 250 ml flask. The exemplary LPC (0.33 grams; IM22-1054, See, Table 1 above) was weighed and added to the flask followed by HSPC (2.41 grams). A 2.5 cm egg-shaped magnet was added to the flask and mixing was performed at 65 °C for 30 minutes until a homogenous clear solution was obtained.
Ethanol evaporation—The solution was evaporated using a ‘BUCHI’ Roto-vapor (water bath set to 35 °C, −2 inclination, 250 RPM) until dry, followed by a second verifying evaporation, forming a dry lipid cake.
Hydration in water—55 grams of di-ionized water at 65 °C were added to the dried flask along with a 2.5 cm magnet and stirring was effected for 1 hour at 65 °C.
Homogenization—The solution was placed inside a cylinder immersed in a 65 °C bath, to be homogenized at 6-9 K RPM for 30 minutes using a narrow ‘Kinematica’ knife, at that temperature. The resulting MLVs DLS analysis showed a Z-average of 420±3 nm, and PDI of 0.304±0.016. See, Table 2, Entry 4, in Example 1 hereinabove.
Gel embedding—12.56 grams of the stock gel was mixed with 50.64 grams of the homogenized solution using an over-head stirrer equipped with a stainless steel ‘anchor’ type rod.

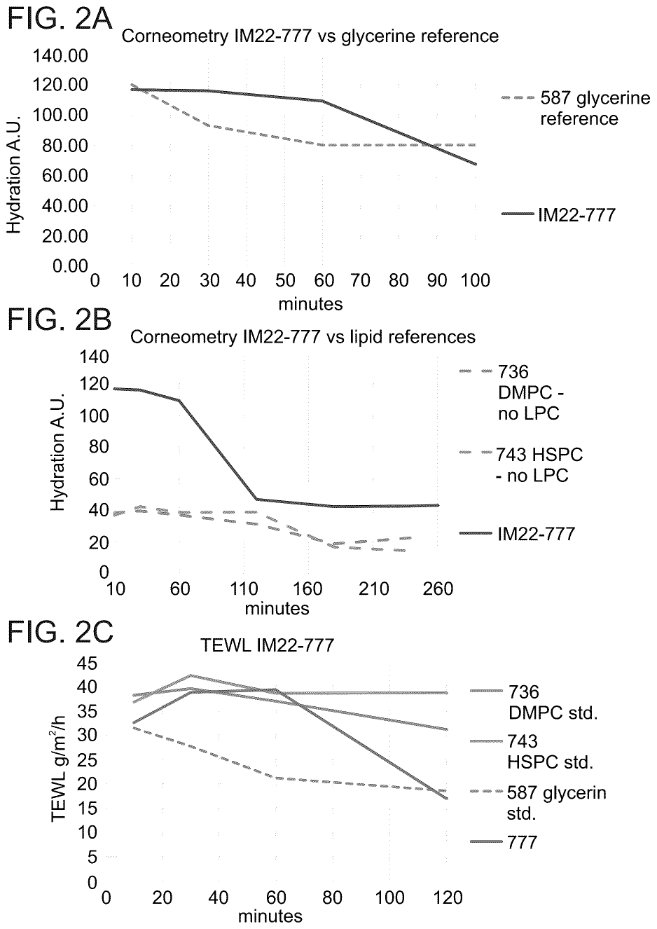

FIGS. 2A-B present graphs showing the corneometry (hydration) of the IM22-777 gel formulation as a function of time in comparison with a glycerin reference IM22-587 (FIG. 2A) and in comparison with various lipid standards (TM22-743 and TM22-736) (FIG. 2B).

FIG. 2C presents the TEWL of IM22-777 as a function of time, in comparison with various lipid standards (IM22-743 and IM22-736), and also in comparison with IM22-587, a glycerin standard.

### A Gel Comprising Liposomes Based on LPC (Long) and HSPC (Also Referred to Herein as Formulation IM22-778; Table 2, Entry 3):

A gel formulation comprising multilamellar vesicles composed of Hydrogenated soy phosphatidylcholine lipids (HSPC), the LPC IM22-1054 (see, Table 1) and water; mixed with Carbomer gel (Carbopol®, di-ionized water, Tri-ethanolamine, and preservatives), resulting in 44.80 mM gel at a 4% w/w final gel lipid concentration was prepared as follows:
Gel preparation—a pre-made gel stock (IM22-728-1.5% Carbopol® content) was prepared by adding 6 grams Carbopol® 990 to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine were added to neutralize the acidic Carbomer to a pH of 6.17.
Ethanol dissolving of the lipids—55 grams Ethanol AR at 65 °C were added to a 250 ml flask. The exemplary LPC (0.52 grams; IM22-1055, See, Table 1 above) was weighed and added to the flask followed by HSPC (2.42 grams). A 2.5 cm egg-shaped magnet was added to the flask and mixing was performed at 65 °C for 10 minutes until a homogenous clear solution was obtained.
Ethanol evaporation—The solution was evaporated using a ‘BUCHI’ Roto-vapor (water bath set to 35 °C, −2 inclination, 250 RPM) until dry, followed by a second verifying evaporation, forming a dry lipid cake.
Hydration in water—55 grams of di-ionized water at 65 °C were added to the dried flask along with a 2.5 cm magnet and stirring was effected for 1 hour at 65 °C.
Homogenization—The solution was placed inside a cylinder immersed in a 65 °C bath, to be homogenized at 7 K RPM for 40 minutes using a narrow ‘Kinematica’ knife, at that temperature. The resulting MLVs DLS analysis is presented in Table 2, Entry 3, in Example 1 hereinabove.
Gel embedding—12.56 grams of the stock gel was mixed with 50.64 grams of the homogenized solution using an over-head stirrer equipped with a stainless steel ‘anchor’ type rod.

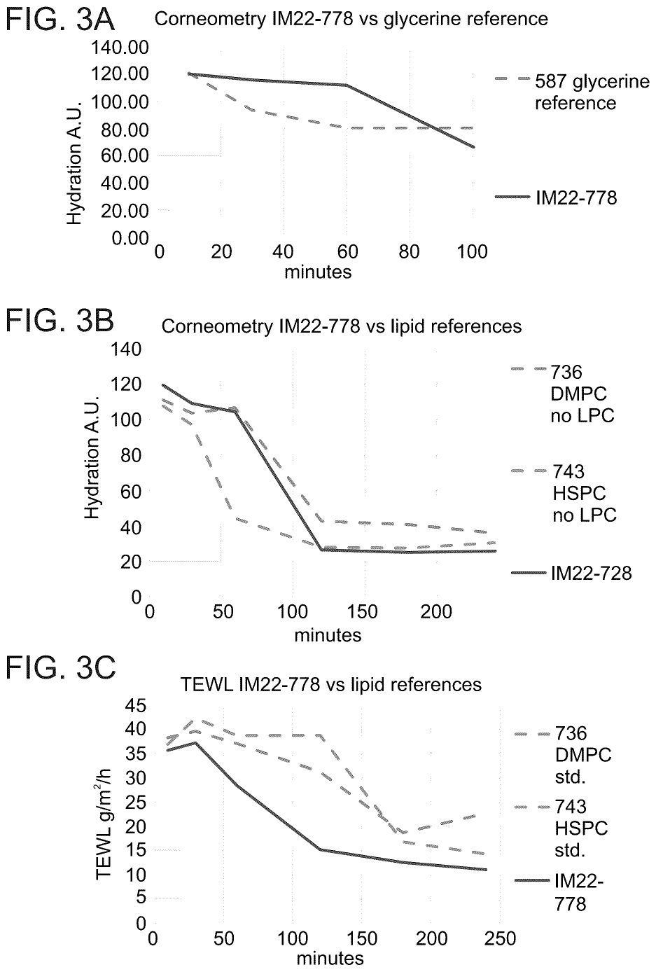

FIGS. 3A-B present graphs showing the corneometry (hydration) of the IM22-778 gel formulation as a function of time in comparison with a glycerin reference IM22-587 (FIG. 3A) and in comparison, with various lipid standards (IM22-743 and IM22-736) (FIG. 3B).

FIG. 3C presents the TEWL of IM22-778 as a function of time, in comparison with various standards.

### A Gel Comprising Liposomes Based on LPC (Long) and DMPC (Also Referred to Herein as Formulation “IM22-754”; Table 2, Entry 5):

A gel formulation comprising multilamellar vesicles composed of dipalmitoyl phosphatidylcholine lipids (DMPC), the LPC IM22-1054 (see, Table 1) and water; mixed with Carbomer gel (Carbopol®, di-ionized water, Tri-ethanolamine, and preservatives), resulting in 102.32 mM gel at a 4% w/w final gel lipid concentration was prepared as follows:
Gel preparation—a pre-made gel stock (IM22-728-1.5% Carbopol® content) was prepared as follows: 6 grams Carbopol® 990 was added to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine was added to neutralize the acidic Carbomer to a pH of 6.17.
Ethanol dissolving of the lipids—55 grams Ethanol AR at 34 °C was added to a 250 ml flask. LPC (IM22-1054 (see, Table 1); 1.51 grams) was added to the flask, followed by DMPC (4.74 grams). A 2.5 cm egg-shaped magnet was added to the flask and mixing was performed at 35 °C until a homogenous clear solution was obtained.
Ethanol evaporation—The solution was evaporated using a ‘BUCHI’ Roto-vapor (water bath set to 40 °C, −2 inclination, 250 RPM) until dry, followed by a second verifying evaporation, forming a dry lipid cake.
Hydration in water—55 grams of di-ionized water at 34 °C were added to the dried flask along with a 2.5 cm magnet and stirring was applied for 2 hours at 34 °C.

The resulting MLVs DLS analysis showed a Z-average of 676.8±41 nm and PDI of 0.622±0.047. See, Table 2, Entry 5 in Example 1 hereinabove.
Gel embedding—12.5 grams of the stock gel was mixed with 50.0 grams of the homogenized solution using an over-head stirrer using a stainless steel ‘anchor’ type rod.

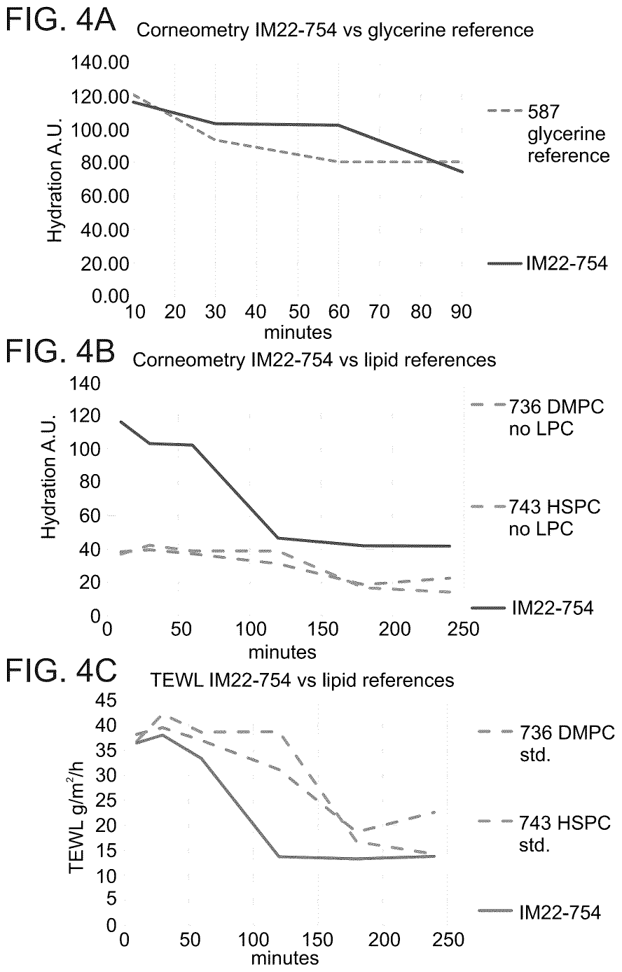

FIGS. 4A-B present graphs showing the corneometry (hydration) of the IM22-754 gel formulation as a function of time in comparison with a glycerin reference IM22-587 (FIG. 4A) and in comparison with various lipid standards (IM22-743 and IM22-736) (FIG. 4B).

FIG. 4C presents the TEWL of IM22-754 as a function of time, in comparison with various standards.

### A Gel Comprising Liposomes Based on LPC (Long) and DMPC (Also Referred to Herein as Formulation “IM22-744”; Table 2, Entry 6):

A gel formulation comprising multilamellar vesicles composed of dipalmitoyl phosphatidylcholine lipids (DMPC), the LPC IM22-1054 (see, Table 1 in Example 1) and water; mixed with Carbomer gel (Carbopol®, di-ionized water, Tri-ethanolamine, and preservatives), resulting in 51.16 mM gel at a 4% w/w final gel lipid concentration was prepared as follows:
Gel preparation—a pre-made gel stock (IM22-728-1.5% Carbopol® content) was prepared as follows: 6 grams Carbopol® 990 was added to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine was added to neutralize the acidic Carbomer to a pH of 6.17.
Ethanol dissolving of the lipids—55 grams Ethanol AR at 34 °C were added to a 250 ml flask. LPC (IM22-1054; 0.76 grams); was added to the flask, followed by DMPC (2.37 grams). A 2.5 cm egg-shaped magnet was added to the flask and mixing was performed at 26 °C for 30 minutes until a homogenous clear solution was obtained.
Ethanol evaporation—The solution was evaporated using a ‘BUCHI’ Roto-vapor (water bath set to 40 °C, −2 inclination, 250 RPM) until dry, followed by a second verifying evaporation, forming a dry lipid cake.
Hydration in water—55 grams of di-ionized water at 26 °C were added to the dried flask along with a 2.5 cm magnet and stirring was performed for 2 hours at 26 °C.

The resulting MLVs DLS analysis showed a Z-average of 983±63.3 nm and PDI of 0.726±0.003. See Table 2, Entry 6, in Example 1 hereinabove.
Gel embedding—12.5 grams of the stock gel were mixed with 50 grams of the homogenized solution using an over-head stirrer equipped with a stainless-steel Type rod.

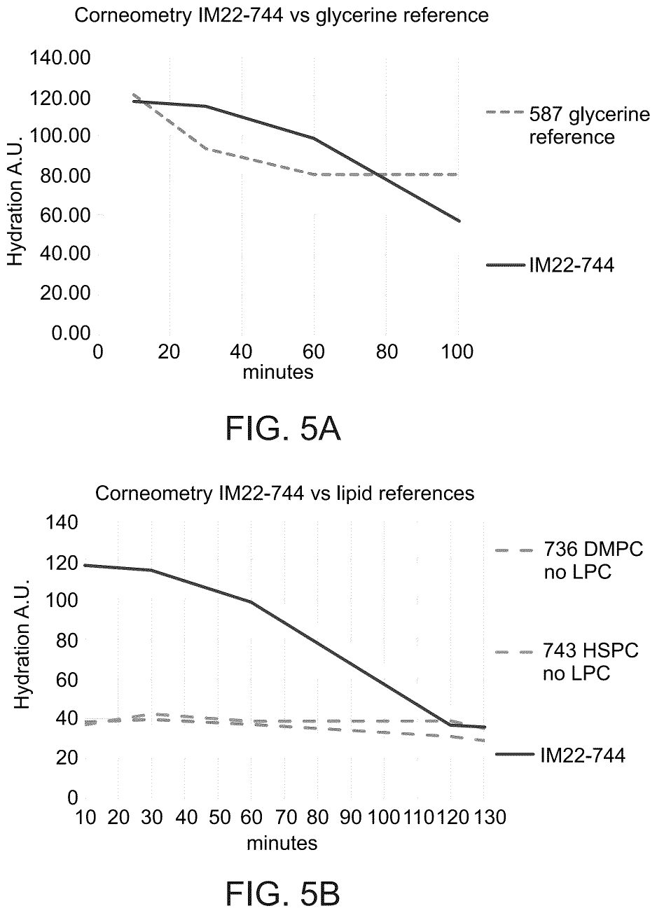

FIGS. 5A-B present graphs showing the corneometry (hydration) of the IM22-754 gel formulation as a function of time in comparison with a glycerin reference IM22-587 (FIG. 5A) and in comparison with various lipid standards (IM22-743 and IM22-736) (FIG. 5B).

### A Gel Comprising Liposomes Based on LPC (Short) and DMPC (Also Referred to Herein as Formulation “IM22-732”; Table 2, Entry 9):

A gel formulation comprising multilamellar vesicles composed of dipalmitoyl phosphatidylcholine lipids (DMPC), the LPC IM22-1053; see, Table 1) and water; mixed with Carbomer gel (Carbopol®, di-ionized water, Tri-ethanolamine, and preservatives), resulting in 54.60 mM gel at a 4% w/w final gel lipid concentration was prepared as follows:
Gel preparation—a pre-made gel stock (IM22-728-1.5% Carbopol® content) was prepared as follows: 6 grams Carbopol® 990 was added to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine was added to neutralize the acidic Carbomer to a pH of 6.17.
Ethanol dissolving of the lipids—55 grams Ethanol AR at 38 °C were added to a 100 ml flask rotating at the Roto-Evaporator. LPC (0.21 grams; IM22-1053) was added to the flask, followed by DMPC (2.53 grams). Mixing was performed at 38 °C for 10 minutes until a homogenous clear solution was obtained.
Ethanol evaporation—The solution was evaporated using a ‘BUCHI’ Roto-vapor (water bath set to 40 °C, −2 inclination, 250 RPM) until dry, followed by a second verifying evaporation, forming a dry lipid cake.
Hydration in water—55 grams of di-ionized water at 37 °C were added to the dried flask along with a 2.5 cm magnet and stirring was effected for 1 hour at 37 °C.
Homogenization—The solution was placed inside a cylinder immersed in a 37 °C bath, homogenized at 9K RPM for 1 hour using a Wide ‘Kinematica’ knife, at that temperature.

The resulting MLVs DLS analysis is shown in Table 2, Entry 9, in Example 1 hereinabove.
Gel embedding—12.54 grams of the stock gel was mixed with about 50 grams of the homogenized solution using an over-head stirrer equipped with a stainless steel ‘anchor’ type rod.

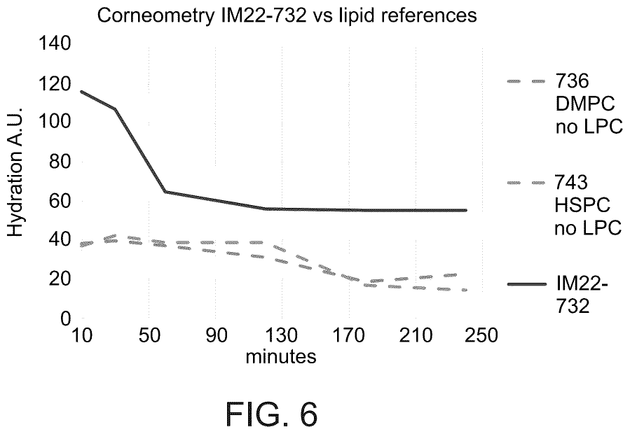

FIG. 6 presents graphs showing the corneometry (hydration) of the IM22-732 gel formulation as a function of time in comparison with various lipid standards (IM22-743 and IM22-736).

### A Gel Comprising Liposomes Based on LPC (Medium) and DMPC (Also Referred to Herein as Formulation “IM22-782”; Table 2, Entry 1):

A gel formulation comprising multilamellar vesicles composed of dipalmitoyl phosphatidylcholine lipids (DMPC), the LPC IM22-1063 (see, Table 1 in Example 1 hereinabove) and water; mixed with Carbomer gel (Carbopol®, di-ionized water, Tri-ethanolamine, and preservatives), resulting in 54.16 mM gel at a 4% w/w final gel lipid concentration was prepared as follows:
Gel preparation—a pre-made gel stock (IM22-728-1.5% Carbopol® content) was prepared as follows: 6 grams Carbopol® 990 was added to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine was added to neutralize the acidic Carbomer to a pH of 6.17.
Ethanol dissolving of the lipids—55 grams ethanol AR at 35 °C was added to a 250 ml flask. The LPC (IM22-1063; 0.24 grams) was added, followed by DMPC (2.52 grams) that was added with a 2.5 cm egg-shaped magnet. Mixing was performed at 35 °C for 10 minutes at the evaporator until complete dissolution was obtained.
Ethanol evaporation—The solution was evaporated using a ‘BUCHI’ Roto-vapor (water bath set to 40 °C, 250 RPM) until dry, followed by a second verifying evaporation, forming a dry lipid cake.
Hydration in water—55 grams of di-ionized water at 35 °C were added to the dried flask which was mixed using a magnet in a flask for 1 hour at 35 °C bath.
Homogenization—using ‘Kinematica’ narrow knife for 1 hour at 8.0 K RPM at 36 °C bath, then foam left to de-bubble for 3 days.

The resulting MLVs DLS analysis is presented in Table 2, Entry 1, in Example 1 hereinabove.

Gel embedding was performed using 12.50 grams of the stock gel mixed with about 49.99 grams of the homogenized solution using an over-head stirrer equipped with a stainless steel ‘anchor’ type rod.

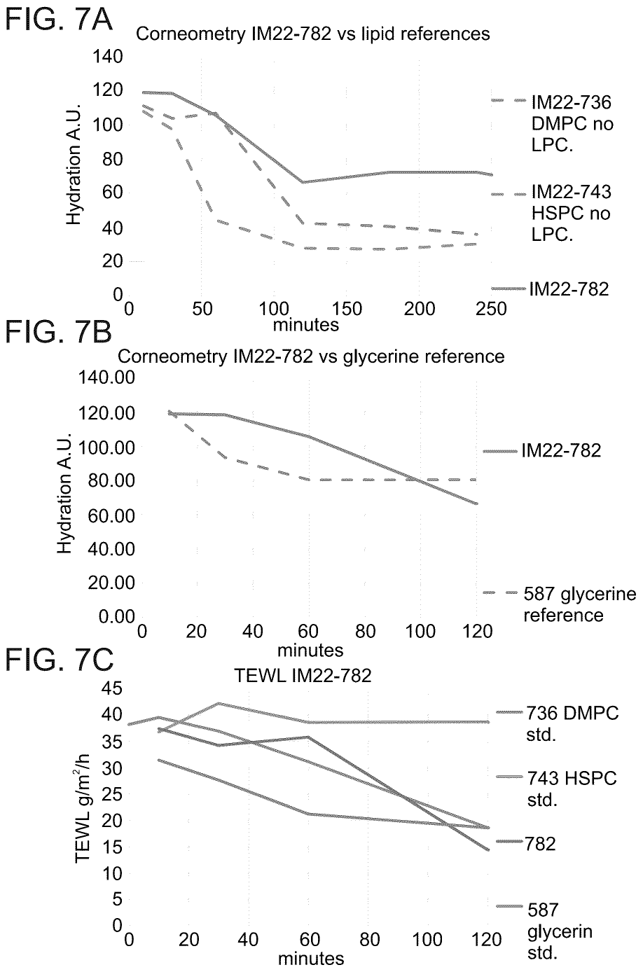

FIGS. 7A-B present graphs showing the corneometry (hydration) of the IM22-782 gel formulation as a function of time in comparison with a glycerin reference IM22-587 (FIG. 7A) and in comparison with various lipid standards (IM22-743 and IM22-736) (FIG. 7B).

FIG. 7C presents the TEWL of IM22-782 as a function of time, in comparison with various standards.

A Gel Comprising Liposomes Based on LPC (Medium) and Phosphatidylcholine from Soybean (PC S 100) (Also Referred to Herein as Formulation “IM22-780”; Table 2, Entry 2:

A gel formulation comprising multilamellar vesicles composed of phosphatidylcholine from soybean (PC S 100), the LPC IM22-1056 (see, Table 1 in Example 1 hereinabove) and water; mixed with Carbomer gel (Carbopol®, di-ionized water, Tri-ethanolamine, and preservatives), resulting in 46.4 mM gel at a 4% w/w final gel lipid concentration, was prepared as follows:
Gel preparation—a pre-made gel stock (IM22-728-1.5% Carbopol® content) was prepared as follows: 6 grams Carbopol® 990 was added to di-ionized water (370 grams) at room temperature and the mixture was homogenized at 9K RPM for 7 minutes, followed by the addition of preservatives (about 16 grams ‘Cosphagard CAP’) and mixing. Thereafter, 6.71 grams of tri-ethanolamine was added to neutralize the acidic Carbomer to a pH of 6.17.
Ethanol dissolving of the lipids—55 grams ethanol AR at 65 °C was added to a 250 ml flask. LPC (0.25 grams; IM22-1056) was added, followed by DMPC (2.50 grams) that was added with a 2.5 cm egg-shaped magnet. Mixing was performed at 65 °C for 30 minutes in the evaporator, until complete dissolution was obtained.

Ethanol evaporation—The solution was evaporated using a ‘BUCHI’ Roto-vapor (water bath set to 60 °C, 250 RPM) until dry, followed by a second verifying evaporation, forming a dry lipid cake.
Hydration in water—55 grams of di-ionized water at 65 °C were added to the dried flask which was mixed using a magnet in a flask for 1 hour at 65 °C bath.

Gel embedding was performed using 12.50 grams of stock gel mixed with about 50.03 grams of the homogenized solution using an over-head stirrer equipped with a stainless steel ‘anchor’ type rod.

The resulting MLVs DLS analysis is presented in Table 2, Entry 2, in Example 1 hereinabove.

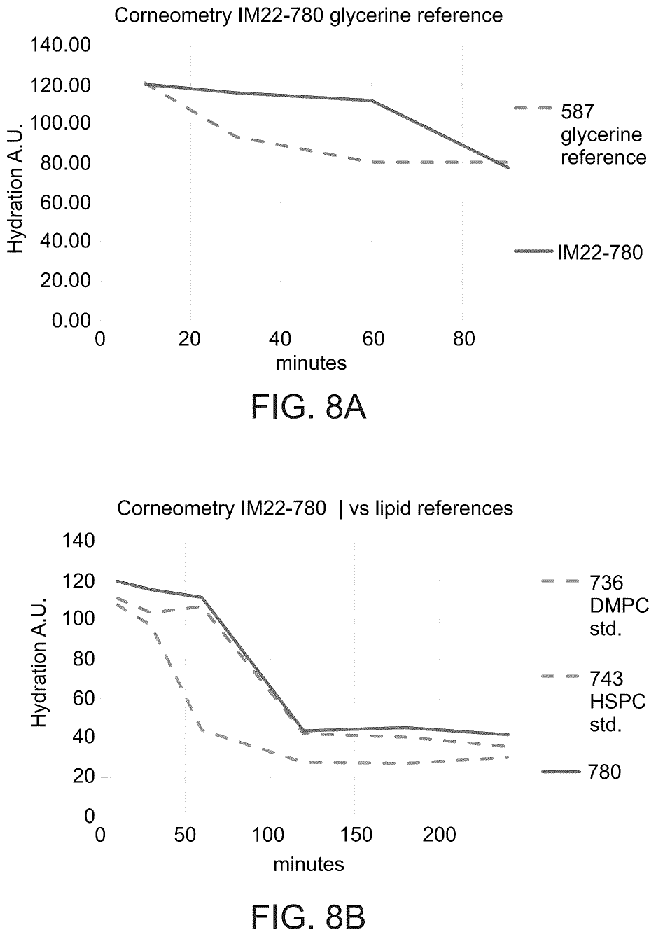

FIGS. 8A-B present graphs showing the corneometry (hydration) of the IM22-780 gel formulation as a function of time in comparison with a glycerin reference IM22-587 (FIG. 8A) and in comparison with various lipid standards (IM22-743 and IM22-736) (FIG. 8B).

Table 3 below summarizes the lipid composition of the above-described gel formulations. “PC lipid” represents the bi-layer forming phospholipid. LPC % w/w refers to the % by weight of the LPC out of the total weight of the formulation.

### Example 3

### Cosmetic Cream Formulations

A cream formulation comprising multilamellar vesicles composed of DMPC and an LPC as described in Example 1 hereinabove, mixed with water, Glycerin, Cetearyl Alcohol (and) Cetearyl Glucoside, Caprylic/Capric Triglyceride, Isononyl Isononanoate, Simmondsia Chinensis (jojoba) seed oil, Butyrospermum Parkii (Shea) Butter, Cocos Nucifera (coconut) oil, sodium polyacrylate, phenoxyethanol, caprylic glycol, tocopheryl acetate and perfume, at a 4 mM lipid concentration, is prepared as follows: Multilamellar vesicles preparation—Multilamellar vesicles (MLVs) are prepared by adding a selected LPC and a selected phospholipid at a selected weight ratio to a flask containing ethanol AR, dissolving the ingredients until fully dissolved, and evaporating at 37 °C bath to form a dry lipid cake. Thereafter, the formed lipid cake is hydrated with di-ionized water and stirred with a magnet for, for example, 1 hour, at, for example, 1500 RPM.
Phase A—water and glycerin are added to a container and heated to 70 °C.
Phase B—5.00% Cetearyl Alcohol (and) Cetearyl Glucoside, 1.70% Caprylic/Capric Triglyceride, 2.70% Isononyl Isononanoate, 0.80% Simmondsia Chinensis (jojoba) seed oil, 0.35% Butyrospermum Parkii (Shea) Butter, 0.20% Cocos Nucifera (coconut) oil are added to a container and heated to 70 °C.
A+B Phase dispersion—phase A is added to phase B with immediate homogenization, for e.g., 10 minutes at e.g., 10-15K RPM, using, e.g., a ‘Kinematica’ wide knife, until formation of one phase. 0.20% sodium polyacrylate is added at a temperature below 60 °C, and the solution is re-homogenized for 5 minutes. The obtained preparation is cooled while stirred using an over-head stirrer to below 40 °C. Thereafter, 10.00% MLVs, 0.80% phenoxyethanol and Caprylyl glycol, 0.10% tocopheryl acetate, and 0.03% perfume are added under stirring.

A short homogenization (2 minutes) is then performed.

### Example 4

### Skin Hydration

### Materials and Methods

Gel formulations were prepared according to Example 2 hereinabove.

The formulations were applied to a human skin. Trans-Epidermal Water Loss (TEWL) & Corneometry measurements were determined using a ‘Courage+Khazaka’ Multi Probe Adapter System MPA 6, using the test procedure provided below. The Trans-Epidermal Water Loss (TEWL) technique is based on measuring the flux of water molecules that evaporates from the treated skin. The procedure of measurement was effected separately for each formulation on three locations on each skin segment three times. The measurements were compared to the naïve skin in order to demonstrate the state where the effect of the treatment vanished. In addition, the effect of the gel formulation was compared to industrial acceptable moisturizers that comprise Hyaluronic Acid, Propylene Glycol, and Glycerin.

Skin hydration was determined also by Corneometry.

Human skin was obtained from an aesthetic abdominal cosmetic surgery, upon consent of the donor (female, age 37). The subcutaneous fat layer was removed, and the skin was kept defatted (full thick) at −20 °C until use (less than 3 months, ensuring its barrier function). The study was conducted on full thickness human skin (epidermis+dermis). Sixteen (16) test items were evaluated in this experiment.

Both TEWL and skin hydration were evaluated by corneometric determination. TEWL is the amount of water that passively evaporated throughout skin to the external environment due to the water vapor pressure gradient on both sides of the skin barrier and is used to characterize skin barrier function. Skin hydration is determined by the epidermal conductivity, which correlates to its water content.

On the day of the experiment, the skin was thawed at RT, and the dermal side was placed at PBS for 30 min for equilibrium. The epidermal side was gently dried and was topically applied (200 ml/cm²) with massage to the applied area. Due to the large amount of test groups, the experiment was conducted during 3 days using the same skin donor, testing a sample and its respective naïve group on the same day.

TEWL and skin hydration (Corneometry) were determined kinetically at 0, 10, 30, 60, 120, 180 and 240-min post application, after electrode calibration, according to the manufacturer's instructions. The pH of the naïve group was measured kinetically in the same time points as mentioned above.

### Data Analysis and Results:

The TEWL measurement is correlative to water leaving the skin, thus lower results mean less water leaving the skin, and better hydration, in respect to the naïve untreated sample.

Corneometry measurements indicate the hydration level of the superficial layers of the skin (stratum corneum) via measurement of skin dielectric properties. The measurements are performed by the application of a probe to the skin surface. Upon contact, an electric field passes through the stratum corneum and the dielectric constant is obtained. The value of the dielectric constant (in arbitrary units) is directly proportional to the level of skin hydration.

The data obtained for exemplary tested formulations are shown in FIGS. 2A-8B, and in Table 4 (TEWL) and Table 5 (Corneometry) below.

TABLE 4
TEWL measurements (g/m²/h)

| | 10 | 30 | 60 | 120 | 180 | 240 |
|---|---|---|---|---|---|---|
| Batch | minutes | minutes | minutes | minutes | minutes | minutes |
| Naïve | 10.9 | 25.7 | 26.3 | 41.1 | 30.5 | 33.8 |
| IM22-744 | 38.6 | 37.7 | 31.7 | 37 | 15.2 | 14.5 |
| IM22-754 | 36.4 | 38 | 33.3 | 13.6 | 13.2 | 13.7 |
| IM22-777 | 32.5 | 38.7 | 39.2 | 17 | 14.3 | 13.7 |
| IM22-778 | 35.5 | 37.1 | 28.3 | 15 | 12.4 | 10.9 |
| IM22-782 | 37.4 | 34.2 | 35.8 | 14.3 | 12.5 | 12.6 |
| IM22-780 | 36.9 | 36 | 38.2 | 17.1 | 14.1 | 12.9 |
| IM22-732 | 29.3 | 30.6 | 38.6 | 34.2 | 21.9 | 31.4 |
| IM22-743 | 36.8 | 42.2 | 38.5 | 38.7 | 16.7 | 14.1 |
| IM22-736 | 38.1 | 39.5 | 36.9 | 31.1 | 18.5 | 22.5 |

TABLE 5
Corneometry measurements

| | 10 | 30 | 60 | 120 | 180 | 240 |
|---|---|---|---|---|---|---|
| | minutes | minutes | minutes | minutes | minutes | minutes |
| Naïve | 64 | 58.9 | 64.5 | 55.8 | 48.6 | 54.7 |
| IM22-744 | 116.8 | 114.3 | 98.3 | 36.6 | 31 | 31.8 |
| IM22-754 | 115.8 | 102.9 | 102.1 | 46.5 | 41.7 | 41.6 |
| IM22-777 | 116.7 | 116 | 109.2 | 46.9 | 42.2 | 42.6 |
| IM22-778 | 119.3 | 109.1 | 104.3 | 26.2 | 24.8 | 25.4 |
| IM22-782 | 118.6 | 118 | 105.4 | 66.2 | 72 | 64.6 |
| IM22-780 | 119.5 | 115.3 | 111.3 | 43.6 | 45.2 | 41.7 |
| IM22-732 | 119.4 | 116 | 107 | 64.7 | 55.2 | 55.1 |
| IM22-743 | 107.6 | 97 | 44.1 | 27.7 | 27.1 | 30.2 |
| IM22-736 | 111 | 103.6 | 106.6 | 42.4 | 40.4 | 35.7 |

The aim of this study was to evaluate the efficacy of the newly designed formulations. Ten (10) formulations were evaluated, along with two formulations (IM22-743 and IM22-736) that served as reference of lipid hydration ability, without LPC.

Skin hydration and TEWL were measured kinetically during 240 min post exposure to the formulations.

As can be seen in the data presented in FIGS. 2A-8B and Tables 4 and 5, in the short exposure time (up to 30 minutes) all formulations enhance the hydration parameter compared to the naïve group levels. At 30 and 60 minutes, a reduction in skin hydration was seen. After 120 minutes, a stable state was seen in all compounds. These findings demonstrate a profound ability of the tested formulations to enhance the barrier properties of the skin and thereby reduce overall water loss, indicating the high efficacy of the formulations.

### Example 5

### Persistence of the Hydration Layer on Synthetic Dry Skin

### Materials and Methods

Two techniques were utilized to measure the hydration persistence: analytical balance (OHAUS EQ-238) and NMR-MOUSE (ACT-Magritek GmbH, Aachen, Germany).

Analytical balance was determined by depositing the gels to fill completely a cell of 3 mm depth with an area of 125.5 mm², and measuring mass loss over time, using NMR-MOUSE. The measured mass loss reveals the evaporation rate from the sample. The rate is extracted from the corresponding slope at the first hour and after 10 minutes from the spreading to reduce the contribution of variations to the mass loss by the manual spreading. At the first hour, for this thickness of deposited gel, the rate (slope) is safely constant and can be attributed to the evaporation of unbound water.

Measurements with the NMR-MOUSE were conducted on porcine skin from ears. Pieces from the ears skin with the dermis and epidermis layers were cut upon arrival and frozen at −20 °C. Before each measurement, a piece was defrosted at room temperature and dried by a blotting paper for several minutes. Then, for some of the samples, measurement was conducted before the deposition of the gel formulations. The spreading of the gel formulations was done with a wood popsicle stick on a marked circular area of 19 mm diameter.

The NMR-MOUSE is equipped with a permanent magnet and surface radiofrequency (RF) coil for transmission and reception. See, Nicasy et al., Polymers 2022, 14, 798; and Bergman et al., NMR Biomed. 2015, 28, 656-666. The U-shaped magnet provides a strong gradient of approximately 8 T/m along the z-direction, away from the face of the magnet. The strong Z gradient, combined with the hard RF pulses, produces a selective excitation of thin, flat slices of a few hundreds of micrometers at a distance of 25 mm from the magnet; the static magnetic field in this 25-mm space is approximately 0.3 T (with a resonance frequency of approximately 13.6 MHz). Cross section diameter of the RF beam was estimated to be 0.6-0.7 mm. Carr-Miligoom-Purcel-Gil (CMPG) train of RF-pulses was utilized to tilt the magnetization by 90° to the lateral plane and probe its echo.

The following formulations were tested:
IM23-587 (5% w/w glycerin in Gel)
IM22-736 (DMPC only in gel),
IM22-732 (DMPC+LPC gel)

### Experimental Steps

Step 1: To verify that protons from water are sampled, a clear signal at v=13.67 MHz from a 5 mM solution of CuSO4 in a glass bottle was acquired.
Step 2: A gel formulation was spread in a covered petri-dish just to find the signal from the protons in the gel. Again, a clear signal was observed at v=13.67 MHz. Note, to optimize the signal acquisition the optimal sample height and RF intensity (when the pulse duration is fixed) had to be determined.
Step 3: A gel was spread on the substrate and covered with nylon to find a-priori the optimized height and save time for the loss rate measurement on the skin.
Step 4: For reference, the plastic substrate was cleaned from the gel with paper and ethanol and the dry skin was tightened to the substrate with strips of red tape, see FIG. 3. Following, the magnetization signal was measured.
Step 5: The loss rate of hydration was measured by spreading the gel on the tightened synthetic skin and after a short signal optimization (1-2 minutes). The loss rate measurement were stopped after two hours.
Step 6: The gel was wiped from the tightened skin with paper and ethanol and the magnetization signal was measured.
Step 7: Another reference sample of dry skin was tightened and the magnetization signal was measured.

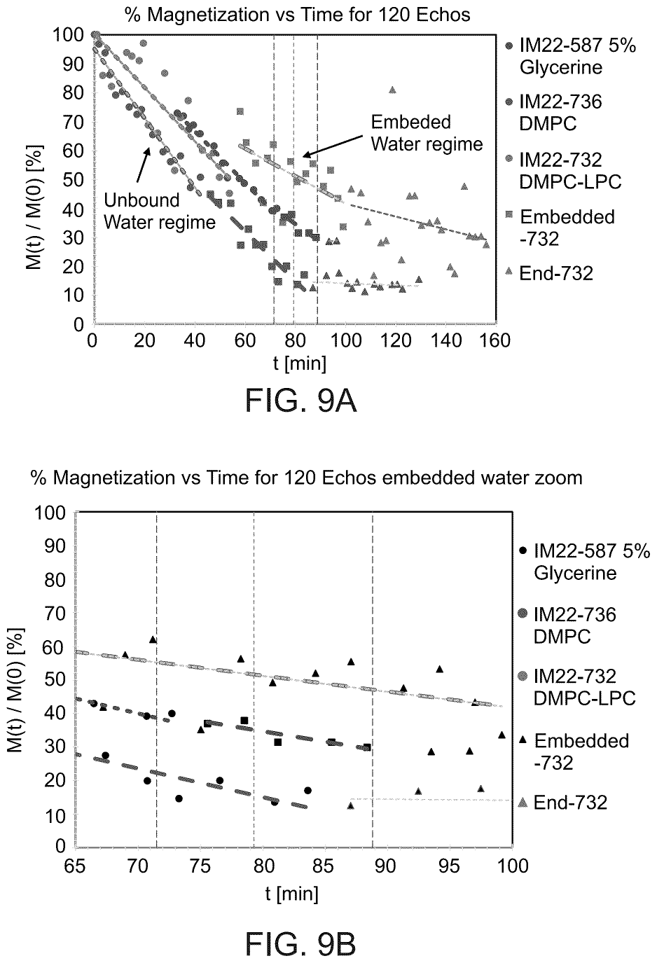

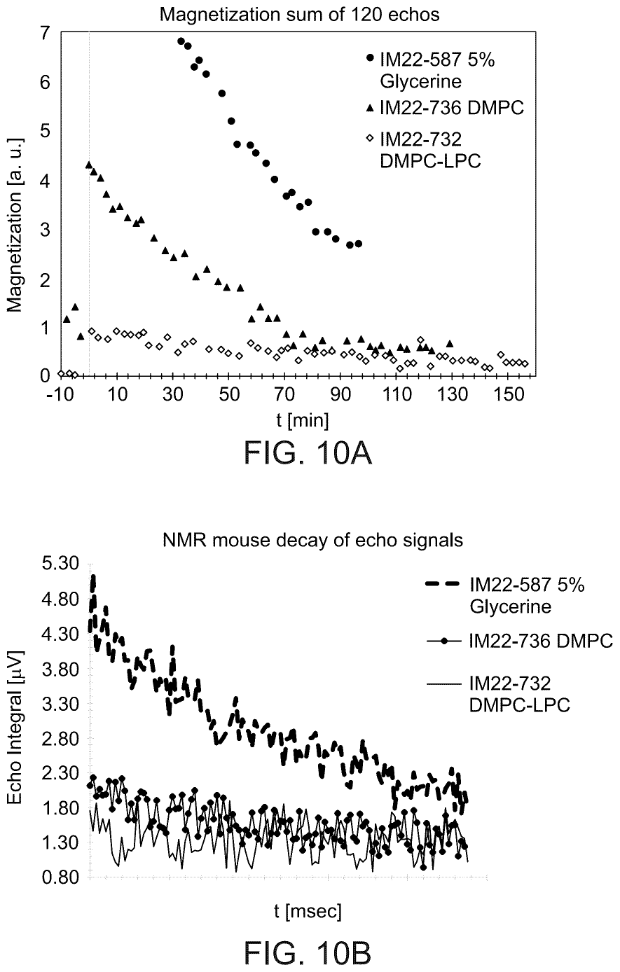

The magnetization signal from protons of water molecules of three samples spread on porcine skin taken from ears is shown in FIG. 10A; negative times correspond to pre-deposition state. The calibration procedure to optimize the signal was conducted during the first 30 minutes after glycerine deposition. The differences in the signal intensity just after deposition cannot be explained by the differences in the amount of deposited water, but rather by the distinct decay of the echo signal as shown in FIG. 10B, that can be attributed to different relaxation time T₂, proton diffusion and exchange processes. This also applies to the higher pre-deposition signal from the DMPC sample relative to the DMPC-LPC. Each curve was therefore normalized to its initial intensity just after deposition, i.e. M(t)/M(0), as seen in FIGS. 9A-B. For the glycerine sample, M(t=0) was derived from the intercept point in the fitted linear equation.

The evolution of the curves in FIGS. 9A-B exhibits three distinct regimes. The measurement values of the first regime, from t=0 minutes to 74/44/56 minutes for Glycerine/DMPC/DMPC-LPC, respectively, is denoted by the circles and the corresponding fitted linear curves (dotted). The reduction of the signal in this regime is predominated by the evaporation process of the unbound (i.e., weakly bound) water molecules.

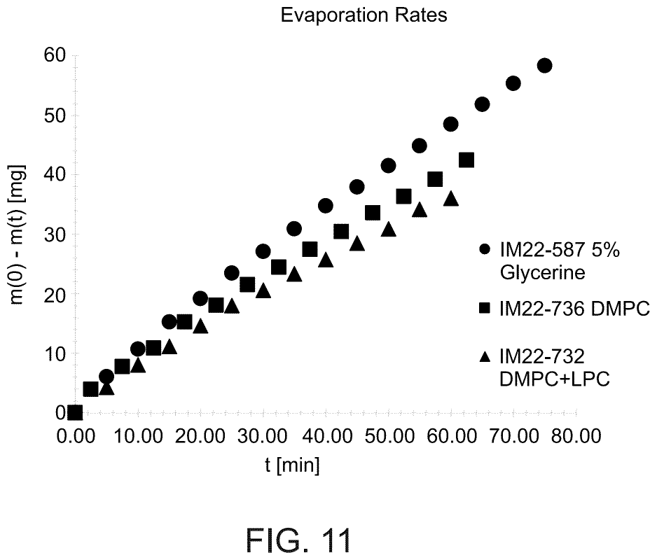

Another way to deduce the evaporation rate of each substance without the influence of the skin is by measuring the weight loss using analytical balance as shown in FIG. 11. Both FIGS. 9A-B and FIG. 11 show curves with different slopes that correspond to the evaporation rate.

The averaged shrinkage rate in units of m/min can be extracted as the cell dimension, deposited volume, spread area, and the duration of evaporation are known. The results are summarized in Table 6 below. The slope values are derived from linear fitting (R²>0.996) to the curves in FIGS. 9A-B, from 10 minutes after deposition (to diminish variations due to the spreading) and up to 60 minutes (to stay safely in the linear regime).

For the evaporation cell on the analytical balance, the slope can be solely related to the evaporation of water. Indeed, the glycerine sample is seen to lose its water most rapidly, whereas the DMPC-LPC maintain the water for the longest time. Same tendency appears in the shrinkage rate that is deducted from the MOUSE-NMR. It is to be noted that in the MOUSE-NMR, other processes such as permeation into the skin and signal decay by the effective T₂ may affect the result. Notwithstanding, it can be seen that the DMPC-LPC treatment consistently has the lowest nominal shrinkage rate, indicating that it persists its unbound water molecules for the longest time.

In FIGS. 9A-B, a change in the slope is seen for all samples which become more moderate before the completion of evaporation. As the evaporation continues, other processes become more dominant due to the concentration increase that affects the water-holding capability and the effective T₂ as well. This indicates that the magnetization technique reveals the regime of bound- or embedded water.

The third regime (denoted by triangles in FIGS. 9A-B) is typified in a plateau for the Glycerine and DMPC samples, and in a wide range of values for the DMPC-LPC sample. It is thus attributed to the state where the level of embedded water is stable.

In FIG. 9B, the embedded-water regime is shown. The slope of the DMPC-LPC (denoted by triangles) is again the most moderate, pointing to the capability of the DMPC-LPC to hold the water for longer times.

The obtained data indicate that measurements conducted according to two techniques indicate the superiority of the DMPC-LPC over the other tested formulations in moisturizing the skin, both in the unbound- and the embedded-water regime.

The glycerin is the smallest in dimensions and as such is expected to have the highest permeation into the skin's stratum corneum layers; a property that is exploited to optical clearance for Raman spectroscopy measurements of skin. On the other hand, the DMPC forms liposomes and multi-lamellar vesicles (MLV) with hydrophilic groups (carbonyl, and phosphate groups) that point radially inward and outward where they bind to water molecules. Their permeation into the skin is expected to be shallower than the glycerine due to their much larger dimension (100 nm-1000 nm). For the DMPC-LPC, the LPC polymer contributes the hydrophilic amide groups in addition to carbonyl and phosphate groups that also attach to water molecules. Its permeation is also expected to be shallower. Furthermore, with the water evaporation, the concentration increases such that in the case of the DMPC-LPC, a thin polymeric film (or polymeric colloids) that enclose the water, is formed. This film effectively traps the water for a longer time period and turns the DMPC-LPC to a superior moisturizer.

TABLE 6

| | | Shrinkage | |
|---|---|---|---|
| | Slope of | rate | |
| Sample | fitted curve | [μm/min] | Method/Regime |
| 5% Glycerine | 0.748 ± 0.046 | 5.96 | Evaporation cell |
| (IM22-587) | [mg/min] | | |
| DMPC | 0.619 ± 0.012 | 4.93 | Evaporation cell |
| (IM22-736) | [mg/min] | | |
| DMPC + LPC | 0.559 ± 0.038 | 4.45 | Evaporation cell |
| (IM22-732) | [mg/min] | | |
| 5% Glycerine | −0.89 | 4.56 | Mouse-MRI/ |
| (IM22-587) | | | Evap. |
| DMPC | −1.20 | 4.52 | Mouse-MRI/ |
| (IM22-736) | | | Evap. |
| DMPC + LPC | −0.93 | 3.26 | Mouse-MRI/ |
| (IM22-732) | | | Evap. |
| 5% Glycerine | −0.62 | — | Mouse-MRI/ |
| (IM22-587) | | | Embed |
| DMPC | −0.86 | — | Mouse-MRI/ |
| (IM22-736) | | | Embed |
| DMPC + LPC | −0.47 | — | Mouse-MRI/ |
| (IM22-732) | | | Embed |

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A cosmetic or cosmeceutical composition (or formulation) comprising a liposome and a cosmetically acceptable carrier, wherein the liposome comprises:
a) at least one bilayer-forming lipid; and
b) a polymeric compound having the general formula I: 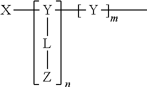
wherein:
m is zero or a positive integer;
n is an integer which is at least 2, at least 5, preferably at least 10 (e.g., of from 10 to 200);
Y is a backbone unit which forms a polymeric backbone of the polymeric compound;
L is absent or is a linking moiety;
Z has the general formula II: 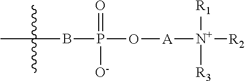
wherein:
the curved line denotes an attachment point to the respective Y backbone unit;
A is a substituted or unsubstituted hydrocarbon;
B is an oxygen atom or is absent;
R₁-R₃ are each independently selected from the group consisting of hydrogen, alkyl, cycloalkyl, heteroalicyclic, aryl and heteroaryl; and
X is a lipid moiety represented by Formula IV: 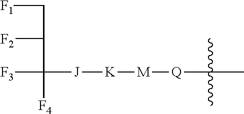
wherein:
F₁, F₂, F₃ and F₄ are each independently selected from the group consisting of hydrogen, alkyl, alkenyl, alkynyl, acyl, alkoxy, thioalkoxy, carboxy, thiocarboxy, wherein at least one of F₁, F₂, F₃ and F₄ is not hydrogen and is of at least 10 carbon atoms in length;
J is —O—P(═O)(OH)—O—or absent;
K is a substituted or unsubstituted hydrocarbon from 1 to 10 carbon atoms in length or absent;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent,
wherein when M is absent, Q is also absent, and when J is absent, M is not absent,
provided that:
when J is —O—P(═O)(OH)—O—, M is other than amido and/or Q comprises an aryl moiety.

2. (canceled)

3. The cosmetic or cosmeceutical composition of claim 1, wherein at least one of F₁, F₂, F₃ and F₄ is derived from a fatty acid selected from the group consisting of lauroyl, myristoyl, palmitoyl, stearoyl, palmitoleoyl, oleoyl, and linoleoyl.

4-5. (canceled)

6. The cosmetic or cosmeceutical composition of claim 1, wherein J is —P(═O)(OH)—O—; M is amido; and Q is a hydrocarbon substituted by at least one aryl (e.g., phenyl).

7. The cosmetic or cosmeceutical composition of claim 1, wherein Q is a methylene substituted by at least one aryl.

8-9. (canceled)

10. The cosmetic or cosmeceutical composition of claim 1, wherein J and K are each absent and M is carboxy.

11. The cosmetic or cosmeceutical composition of claim 1, wherein at least one, or at least two, of F₁, F₂, F₃ and F₄ is independently said thioalkoxy and/or said carboxy.

12-13. (canceled)

14. The cosmetic or cosmeceutical composition of claim 1, wherein Y is a substituted or unsubstituted alkylene unit.

15. (canceled)

16. The cosmetic or cosmeceutical composition of claim 1, wherein:
B is an oxygen atom; and/or
A is a substituted or unsubstituted hydrocarbon from 1 to 4 carbon atoms in length; and/or
R₁-R₃ are each independently hydrogen or C₁₋₄-alkyl.

17-21. (canceled)

22. The cosmetic or cosmeceutical composition of claim 1, wherein n is at least 30.

23. (canceled)

24. The cosmetic or cosmeceutical composition of claim 1, wherein n is at least 50.

25-28. (canceled)

29. The cosmetic or cosmeceutical composition of claim 1, wherein at least a portion of said backbone units Y, said L and/or said Z comprise at least one targeting moiety.

30. The cosmetic or cosmeceutical composition of claim 1, wherein a mol ratio of said bilayer-forming lipid and said polymeric compound is in a range of from 5:1 to 5,000:1, or from 10:1 to 1,000:1, or 10:1 to 100:1 or from 10:1 to 50:1, or from 100:1 to 200:1; and/or
a weight ratio of said bilayer-forming lipid and said polymeric compound is in a range of from 1:1 to 3,000:1, or from 1:1 to 1,000:1, or from 1:1 to 500:1, or from 1:1 to 300:1, or from 1:1 to 100:1, or from 1:1 to 50:1, or from 5:1 to 50:1, or from 5:1 to 30:1.

31. The cosmetic or cosmeceutical composition of claim 1, comprising a plurality of liposomes, wherein at least a portion of said liposomes comprises a plurality of liposomes as defined in claim 1.

32. The cosmetic or cosmeceutical composition of claim 31, wherein:
a polydispersity index of said liposomes is lower than 1, or lower than 0.8, or lower than 0.6; and/or
a mean diameter of said liposomes is in a range of from 50 to 5,000 nm, or from 500 to 5,000 nm, or from 500 to 3500 nm, or from 200 to 2,000 nm, or from 200 to 1,000 nm, or from 400 to 1,000 nm, or from 400 to 800 nm.

33. (canceled)

34. The cosmetic or cosmeceutical composition of claim 1, wherein an amount of said bilayer-forming lipid in the composition in a range of from 0.1 to 5, % by weight of the total weight of the composition.

35. (canceled)

36. The cosmetic or cosmeceutical composition of claim 1, being formulated for topical application.

37-38. (canceled)

39. The cosmetic or cosmeceutical composition of claim 1, wherein said carrier forms a composition is a form of a cream, an ointment, a gel, a lotion, a soap, a shampoo, a water-in-oil emulsion, an oil-in-water emulation, a water-in-oil-in water emulation, an oil-in water-in oil emulsion.

40-41. (canceled)

42. A skin care product comprising the cosmetic or cosmeceutical composition of claim 1.

43-44. (canceled)

45. A method of performing a cosmetic care in a subject in need thereof, the method comprising applying to a skin surface of the subject an effective amount of the cosmetic composition of claim 1, thereby performing the cosmetic care.

46. The method of claim 45, wherein said cosmetic care comprises skin hydration and/or treatment of a damaged keratinous tissue.

47. A method of performing a cosmetic care in a subject in need thereof, the method comprising applying to a skin surface of the subject the skin product of claim 42, thereby performing the cosmetic care.
